(19) United States
(12) Patent Application Publication
(10) Pub. No.: US 2007/0287707 A1
(43) Pub. Date: Dec. 13, 2007

(54) Phosphodiesterase 10 inhibitors

(76) Inventors: Mark P. Arrington, Westwood, NJ (US); Allen T. Hopper, Mahwa, NJ (US); Richard D. Conticello, Cortlandt Manor, NY (US); Hans-Jurgen Hess, Old Lyme, CT (US); Stephen A. Hitchcock, Westlake Village, CA (US)

Correspondence Address: JONES DAY, 222 EAST 41ST ST, NEW YORK, NY, 10017, US

(21) Appl. No.: 11/712,264

(22) Filed: Feb. 27, 2007

Related U.S. Application Data
(60) Provisional application No. 60/778,015, filed on Feb. 28, 2006.

(51) Int. Cl. A61K 31/498; A61K 31/5377; A61P 25/18; C07D 237/28; C07D 413/02

(52) U.S. Cl. 514/235.2; 514/248; 544/116; 544/235

(57) ABSTRACT

The present invention if directed to certain cinnoline compounds that are PDE10 inhibitors, pharmaceutical compounds containing the same and processes for preparing the same. The invention is also directed to methods of treating diseases mediated by PDE10 enzyme such as obesity, non-insulin dependent diabetes, schizophrenia or bipolar disorder, obsessive-compulsive disorder, and the like.

### CROSS-REFERENCE

This application claims the benefit of U.S. Provisional Application No. 60/778,015, filed Feb. 28, 2006, the disclosure of which is incorporated herein by reference in its entirety.

### FIELD OF THE INVENTION

The present invention is directed to certain cinnoline compounds that are PDE10 inhibitors, pharmaceutical compositions containing such compounds and processes for preparing such compounds. This invention is also directed to methods of treating diseases treatable by inhibition of PDE10 enzyme, such as obesity, non-insulin dependent diabetes, schizophrenia, bipolar disorder, obsessive-compulsive disorder, and the like.

### BACKGROUND

Neurotransmitters and hormones, as well as other types of extracellular signals such as light and odors, create intracellular signals by altering the amounts of cyclic nucleotide monophosphates (cAMP and cGMP) within cells. These intracellular messengers alter the functions of many intracellular proteins. Cyclic AMP regulates the activity of cAMP-dependent protein kinase (PKA). PKA phosphorylates and regulates the function of many types of proteins, including ion channels, enzymes, and transcription factors. Downstream mediators of cGMP signaling also include kinases and ion channels. In addition to actions mediated by kinases, cAMP and cGMP bind directly to some cell proteins and directly regulate their activity.

Cyclic nucleotides are produced from the actions of adenylyl cyclase and guanylyl cyclase which convert ATP to cAMP and GTP to cGMP. Extracellular signals, often through the actions of G protein-coupled receptors, regulate the activity of the cyclases. Alternatively, the amount of cAMP and cGMP may be altered by regulating the activity of the enzymes that degrade cyclic nucleotides. Cell homeostasis is maintained by the rapid degradation of cyclic nucleotides after stimulus-induced increases. The enzymes that degrade cyclic nucleotides are called 3′,5′-cyclic nucleotide-specific phosphodiesterases (PDEs).

Eleven PDE gene families (PDE1-PDE11) have been identified so far, based on their distinct amino acid sequences, catalytic and regulatory characteristics, and sensitivity to small molecule inhibitors. These families are coded for by 21 genes; and further multiple splice variants are transcribed from many of these genes. Expression patterns of each of the gene families are distinct. PDEs differ with respect to their affinity for cAMP and cGMP. Activities of different PDEs are regulated by different signals. For example, PDE 1 is stimulated by Ca²⁺/calmodulin. PDE 2 activity is stimulated by cGMP. PDE 3 is inhibited by cGMP. PDE 4 is cAMP specific and is specifically inhibited by rolipram. PDE 5 is cGMP-specific. PDE6 is expressed in retina.

PDE10 sequences were identified by using bioinformatics and sequence information from other PDE gene families (Fujishige et al., J. Biol. Chem. 274:18438-18445, 1999; Loughney et al., Gene 234:109-117, 1999; Soderling et al., Proc. Natl. Acad. Sci. USA 96:7071-7076, 1999). The PDE10 gene family is distinguished based on its amino acid sequence, functional properties and tissue distribution. The human PDE10 gene is large, over 200 kb, with up to 24 exons coding for each of the splice variants. The amino acid sequence is characterized by two GAF domains (which bind cGMP), a catalytic region, and alternatively spliced N and C termini. Numerous splice variants are possible because of at least three alternative exons encoding N termini and two exons encoding C-termini. PDE10A1 is a 779 amino acid protein that hydrolyzes both cAMP and cGMP. The Kₘ values for cAMP and cGMP are 0.05 and 3.0 micromolar, respectively. In addition to human variants, several variants with high homology have been isolated from both rat and mouse tissues and sequence banks.

PDE10 RNA transcripts were initially detected in human testis and brain. Subsequent immunohistochemical analysis revealed that the highest levels of PDE10 are expressed in the basal ganglia. Specifically, striatal neurons in the olfactory tubercle, caudate nucleus and nucleus accumbens are enriched in PDE10. Western blots did not reveal the expression of PDE10 in other brain tissues, although immunoprecipitation of the PDE10 complex was possible in hippocampal and cortical tissues. This suggests that the expression level of PDE10 in these other tissues is 100-fold less than in striatal neurons. Expression in hippocampus is limited to the cell bodies, whereas PDE10 is expressed in terminals, dendrites and axons of striatal neurons.

The tissue distribution of PDE10 indicates that PDE10 inhibitors can be used to raise levels of cAMP and/or cGMP within cells that express the PDE10 enzyme, especially neurons that comprise the basal ganglia and therefore would be useful in treating a variety of neuropsychiatric conditions involving the basal ganglia such as obesity, non-insulin dependent diabetes, schizophrenia, bipolar disorder, obsessive compulsive disorder, and the like.

### SUMMARY OF THE INVENTION

In one aspect, provided herein is a compound of Formula (I): 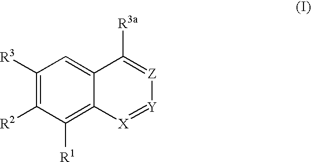
or an individual stereoisomer, a mixture of stereoisomers, or a pharmaceutically acceptable salt thereof, wherein:
Y and Z are nitrogen and X is —CR═ (where R is hydrogen, alkyl, halo, or cyano); or X and Y are nitrogen and Z is —CH═; or X and Z are nitrogen and Y is ═CH—;
One of R¹, R², and R³ is cycloalkyloxy, cycloalkylalkyloxy, hydroxyalkyl, hydroxyalkyloxy, alkoxyalkyl, alkoxyalkyloxy, -(alkylene)NR¹³R¹⁴ or —O-(alkylene)NR¹⁵R¹⁶, wherein R¹³, R¹⁴, R¹⁵, and R¹⁶ are independently hydrogen or alkyl, and wherein one or two carbon atoms in the alkyl in hydroxyalkyl, hydroxyalkyloxy, alkoxyalkyl, alkoxyalkyloxy, -(alkylene)NR¹³R¹⁴ or —O-(alkylene)NR¹⁵R¹⁶ are optionally replaced by one to two oxygen or nitrogen atom(s), and
the other two of R¹, R², and R³ are independently selected from hydrogen, alkyl, alkoxy, cycloalkyl, halo, haloalkyl, haloalkoxy, cyano, hydroxy, carboxy, alkoxycarbonyl, amino, alkylamino, dialkylamino, alkylcarbonyl, cycloalkyl, cycloalkyloxy, cycloalkylalkyloxy, hydroxyalkyl, hydroxyalkyloxy, alkoxyalkyl, alkoxyalkyloxy, -(alkylene)NR¹⁷R¹⁸ or —O-(alkylene)NR¹⁹R²⁰, wherein R¹⁷, R¹⁸, R¹⁹, and R²⁰ are independently hydrogen or alkyl, and wherein one or two carbon atoms in the alkyl in hydroxyalkyl, alkoxyalkyl, -(alkylene)NR¹⁷R¹⁸ or —O-(alkylene)NR¹⁹R²⁰ are optionally replaced by one to two oxygen or nitrogen atom(s);
R^{3a} is aryl, heteroaryl, or heterocyclyl ring substituted with:
   R⁴ where R⁴ is hydrogen, alkyl, halo, haloalkyl, haloalkoxy, cycloalkyl, cycloalkylalkyl, aryl, heteroaryl, heterocyclyl, aralkyl, heteroaralkyl, heterocyclylalkyl, or —X¹R⁷ (where X¹ is —O—, —CO—, —C(O)O—, —OC(O)—, —NR⁸CO—, —CONR⁹—, —NR¹⁰—, —S—, —SO—, —SO₂—, —NR¹¹SO₂—, or —SO₂NR¹²— where R⁸, R⁹, R¹⁰, R¹¹ and R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl); and
   R⁵ and R⁶ where R⁵ and R⁶ are independently hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl;
   and wherein the aromatic or alicyclic ring in R⁴, R⁵, R⁶, and R⁷ is optionally substituted with one to three substituents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substituents independently selected from R^{d} and R^{e} where R^{d} and R^{e} are hydrogen or fluoro;
provided that:
(i) when X and Z are nitrogen, R¹ is hydrogen, R² is alkoxy, alkoxyalkyloxy (wherein one or two carbon atoms in alkoxyalkyloxy are optionally replaced by one to two oxygen atoms), hydroxyalkoxy, or —O-(alkylene)-NR¹³R¹⁴ where R¹³ and R¹⁴ are independently hydrogen or alkyl, and R³ is hydrogen, alkoxy, alkoxyalkyloxy, or hydroxyalkyloxy, then R^{3a} is not
2,3-dihydroindolyl, 2-oxoindolyl, indolyl, 7-aza-2-oxo-indol-3-yl, 4-aza-2-oxo-indol-3-yl, 5,7-diazaoxindol-3-yl, or piperidinyl, each of which is substituted with R⁴, R⁵ or R⁶ as defined above;
6-chloro-7-aza-2-oxo-indol-3-yl; 2-alkyl-5H-pyrrolo[2,3-d]pyrimidin-6(7H)-one-5-yl; 4-carboxypiperidin-1-yl; or
piperazin-1-yl substituted with R⁴, R⁵ or R⁶ at the 4-position of the piperazin-1-yl ring where R⁴, R⁵ or R⁶ are as defined above or where R⁴, R⁵ or R⁶ are hydrogen, alkoxycarbonyl, or —CONHR where R is phenyl substituted with alkoxy, cyano, alkyl, 5-hydroxyindol-1-yl, or cyclopropyl;
(ii) when X and Z are nitrogen, R¹ is hydrogen, R² is cycloalkylpropoxy, R³ is alkoxy, then and R^{3a} is not piperazin-1-yl substituted with R⁴, R⁵ or R⁶ where two of R⁴, R⁵ or R⁶ are hydrogen and the other of R⁴, R⁵ or R⁶ is at the 4-position of the piperazin-1-yl ring and is hydrogen or —CONHR where R is phenyl substituted with alkoxy; and
(iii) when X and Z are nitrogen, R¹ is hydrogen, R² is 2-(dimethylamino)ethoxy, and R³ is methoxy, then R^{3a} is not 1,6-dimethyl-2,4-dioxo-1,2-dihydroquinazolin-3(4H)-yl-piperidin-1-yl;
(iv) or a salt of (i)-(iii).

In a second aspect, this invention is directed to a pharmaceutical composition comprising a compound of Formula (I) or a pharmaceutically acceptable salt thereof and a pharmaceutically acceptable excipient.

In a third aspect, this invention is directed to a method of treating a disorder treatable by inhibition of PDE10 enzyme in a patient which method comprises administering to the patient a pharmaceutical composition comprising a compound of Formula (I) or a pharmaceutically acceptable salt thereof and a pharmaceutically acceptable excipient. Preferably, the disease is obesity, non-insulin dependent diabetes, Huntington's disease, schizophrenia, bipolar disorder, or obsessive-compulsive disorder.

It will be readily apparent to a person skilled in the art that the pharmaceutical composition could contain one or more compounds of Formula (I) (including individual stereoisomer, mixtures of stereoisomers where the compound of Formula (I) has a stereochemical centre), a pharmaceutically acceptable salt thereof, or mixtures thereof.

### DETAILED DESCRIPTION OF THE INVENTION

Definitions

Unless otherwise stated, the following terms used in the specification and claims are defined for the purposes of this application and have the following meanings.

“Alkyl” means a linear saturated monovalent hydrocarbon radical of one to six carbon atoms or a branched saturated monovalent hydrocarbon radical of three to six carbon atoms, e.g., methyl, ethyl, propyl, 2-propyl, butyl (including all isomeric forms), pentyl (including all isomeric forms), and the like.

“Alicyclic” means a non-aromatic ring, e.g., cycloalkyl or heterocyclyl ring.

“Alkylene” means a linear saturated divalent hydrocarbon radical of one to six carbon atoms or a branched saturated divalent hydrocarbon radical of three to six carbon atoms unless otherwise stated, e.g., methylene, ethylene, propylene, 1-methylpropylene, 2-methylpropylene, butylene, pentylene, and the like.

“Alkylthio” means a —SR radical where R is alkyl as defined above, e.g., methylthio, ethylthio, and the like.

“Alkylsulfinyl” means a —SOR radical where R is alkyl as defined above, e.g., methylsulfinyl, ethylsulfinyl, and the like.

“Alkylsulfonyl” means a —SO₂R radical where R is alkyl as defined above, e.g., methylsulfonyl, ethylsulfonyl, and the like.

“Amino” means a —NH₂.

“Alkylamino” means a —NHR radical where R is alkyl as defined above, e.g., methylamino, ethylamino, propylamino, or 2-propylamino, and the like.

“Alkoxy” means an —OR radical where R is alkyl as defined above, e.g., methoxy, ethoxy, propoxy, or 2-propoxy, n-, iso-, or tert-butoxy, and the like.

“Alkoxycarbonyl” means a —C(O)OR radical where R is alkyl as defined above, e.g., methoxycarbonyl, ethoxycarbonyl, and the like.

“Alkoxyalkyl” means a linear monovalent hydrocarbon radical of one to six carbon atoms or a branched monovalent hydrocarbon radical of three to six carbons substituted with at least one alkoxy group, preferably one or two alkoxy groups, as defined above, e.g., 2-methoxyethyl, 1-, 2-, or 3-methoxypropyl, 2-ethoxyethyl, and the like.

“Alkoxyalkyloxy” means an —OR radical where R is alkoxyalkyl as defined above, e.g., methoxyethoxy, 2-ethoxyethoxy, and the like.

“Aminoalkyl” means a linear monovalent hydrocarbon radical of one to six carbon atoms or a branched monovalent hydrocarbon radical of three to six carbons substituted with at least one, preferably one or two, —NRR′ where R is hydrogen, alkyl, or —COR^{a} where R^{a} is alkyl, and R′ is selected from hydrogen, alkyl, hydroxyalkyl, alkoxyalkyl, aryl, aralkyl, heteroaryl, heteroaralkyl, or haloalkyl, each as defined herein, e.g., aminomethyl, methylaminoethyl, 2-ethylamino-2-methylethyl, 1,3-diaminopropyl, dimethylaminomethyl, diethylaminoethyl, acetylaminopropyl, and the like.

“Aminoalkoxy” means an —OR radical where R is aminoalkyl as defined above, e.g., 2-aminoethoxy, 2-dimethylaminopropoxy, and the like.

“Aminocarbonyl” means a —CONRR′ radical where R is independently hydrogen, alkyl, hydroxyalkyl, alkoxyalkyl, or aminoalkyl and R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, or aminoalkyl, each as defined above, e.g., —CONH₂, methylaminocarbonyl, 2-dimethylaminocarbonyl, and the like.

“Aminosulfinyl” means a —SONRR′ radical where R is independently hydrogen, alkyl, hydroxyalkyl, alkoxyalkyl, or aminoalkyl and R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, or aminoalkyl, each as defined above, e.g., —CONH₂, methylaminosulfinyl, 2-dimethylaminosulfinyl, and the like.

“Aminosulfonyl” means a —SO₂NRR′ radical where R is independently hydrogen, alkyl, hydroxyalkyl, alkoxyalkyl, or aminoalkyl and R′ is hydrogen, alkyl, cycloalkyl, cycloalkylalkyl, aryl, aralkyl, heteroaryl, heteroaralkyl, heterocyclyl, heterocyclylalkyl, hydroxyalkyl, alkoxyalkyl, or aminoalkyl, each as defined above, e.g., —SO₂NH₂, methylaminosulfonyl, 2-dimethylaminosulfonyl, and the like.

“Acyl” means a —COR radical where R is alkyl, haloalkyl, cycloalkyl, cycloalkylalkyl, aryl, aralkyl, heteroaryl, heteroaralkyl, heterocyclyl, or heterocyclylalkyl, each as defined above, e.g., acetyl, propionyl, benzoyl, pyridinylcarbonyl, and the like. When R is alkyl, the radical is also referred to herein as alkylcarbonyl.

“Acylamino” means a —NHCOR radical where R is alkyl, haloalkyl, cycloalkyl, cycloalkylalkyl, aryl, aralkyl, heteroaryl, heteroaralkyl, heterocyclyl, or heterocyclylalkyl, each as defined above, e.g., acetylamino, propionylamino, and the like.

“Aryl” means a monovalent monocyclic or bicyclic aromatic hydrocarbon radical of 6 to 12 ring atoms, e.g., phenyl or naphthyl.

“Aralkyl” means an -(alkylene)-R radical where R is aryl as defined above.

“Cycloalkyl” means a cyclic saturated monovalent bridged or non-bridged hydrocarbon radical of three to ten carbon atoms, e.g., cyclopropyl, cyclobutyl, cyclopentyl, cyclohexyl, or adamantyl.

“Cycloalkylalkyl” means an -(alkylene)-R radical where R is cycloalkyl as defined above; e.g., cyclopropylmethyl, cyclobutylmethyl, cyclopentylethyl, or cyclohexylmethyl, and the like.

“Cycloalkyloxy” means an —OR radical where R is cycloalkyl as defined above. Exemplary cycloalkyloxy groups include, for instance, cyclopropyloxy, cyclobutyloxy, cyclopentyloxy, cyclohexyloxy, and the like.

“Cycloalkylalkyloxy” means an —OR radical where R is cycloalkylalkyl as defined above. Exemplary cycloalkylalkyloxy groups include, for instance, cyclopropylmethyloxy, cyclobutylmethyloxy, cyclopentylethyloxy, cyclohexylmethyloxy, and the like.

“Carboxy” means —COOH.

“Disubstituted amino” means a —NRR′ radical where R and R′ are independently alkyl, cycloalkyl, cycloalkylalkyl, acyl, sulfonyl, aryl, aralkyl, heteroaryl, heteroaralkyl, heterocyclyl, heterocyclylalkyl, hydroxyalkyl, alkoxyalkyl, or aminoalkyl, as defined above, e.g., dimethylamino, phenylmethylamino, and the like. When R and R′ are alkyl, the radical is also referred to herein as dialkylamino.

“Halo” means fluoro, chloro, bromo, and iodo, preferably fluoro or chloro.

“Haloalkyl” means alkyl substituted with one or more halogen atoms, preferably one to five halogen atoms, preferably fluorine or chlorine, including those substituted with different halogens, e.g., —CH₂Cl, —CF₃, —CHF₂, —CF₂CF₃, —CF(CH₃)₃, and the like.

“Haloalkoxy” means an —OR radical where R is haloalkyl as defined above, e.g., —OCF₃, —OCHF₂, and the like.

“Hydroxyalkyl” means a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Representative examples include, but are not limited to, hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

“Hydroxyalkoxy” or “hydroxyalkyloxy” means an —OR radical where R is hydroxyalkyl as defined above.

“Heterocyclyl” means a saturated or unsaturated monovalent monocyclic group of 4 to 8 ring atoms in which one or two ring atoms are heteroatom independently selected from N, O, and S(O)ₙ, where n is an integer from 0 to 2, the remaining ring atoms being C. Additionally, one or two ring carbon atoms can optionally be replaced by a —CO— group and the heterocyclic ring may be fused to phenyl or heteroaryl ring provided that the ring is not completely aromatic. Unless stated otherwise, the fused heterocyclyl ring can be attached at any ring atom. More specifically the term heterocyclyl includes, but is not limited to, pyrrolidino, piperidino, homopiperidino, 2-oxopyrrolidinyl, 2-oxopiperidinyl, morpholino, piperazino, tetrahydropyranyl, thiomorpholino, and the like. When the heterocyclyl ring has five, six or seven ring atoms and is not fused to phenyl or heteroaryl ring, it is referred to herein as “monocyclic five- six-, or seven membered heterocyclyl ring or five- six-, or seven membered heterocyclyl ring”. When the heterocyclyl ring is unsaturated it can contain one or two ring double bonds provided that the ring is not aromatic.

“Heterocyclylalkyl” means an -(alkylene)-R radical where R is heterocyclyl ring as defined above, e.g., tetraydrofuranylmethyl, piperazinylmethyl, morpholinylethyl, and the like.

“Heteroaryl” means a monovalent monocyclic or bicyclic aromatic radical of 5 to 10 ring atoms where one or more, preferably one, two, or three, ring atoms are heteroatom independently selected from N, O, and S, the remaining ring atoms being carbon.

“Heteroaralkyl” means an -(alkylene)-R radical where R is heteroaryl as defined above.

“Monosubstituted amino” means an —NHR radical where R is alkyl, cycloalkyl, cycloalkylalkyl, acyl, sulfonyl, aryl, aralkyl, heteroaryl, heteroaralkyl, heterocyclyl, heterocyclylalkyl, hydroxyalkyl, alkoxyalkyl, or aminoalkyl, each as defined above, e.g., methylamino, 2-phenylamino, hydroxyethylamino, and the like.

The present invention also includes the prodrugs of compounds of Formula (I). The term prodrug is intended to represent covalently bonded carriers, which are capable of releasing the active ingredient of Formula (I) when the prodrug is administered to a mammalian subject. Release of the active ingredient occurs in vivo. Prodrugs can be prepared by techniques known to one skilled in the art. These techniques generally modify appropriate functional groups in a given compound. These modified functional groups however regenerate original functional groups in vivo or by routine manipulation. Prodrugs of compounds of Formula (I) include compounds wherein a hydroxy, amino, carboxylic, or a similar group is modified. Examples of prodrugs include, but are not limited to esters (e.g., acetate, formate, and benzoate derivatives), carbamates (e.g., N,N-dimethylaminocarbonyl) of hydroxy or amino functional groups in compounds of Formula (I)), amides (e.g., trifluoroacetylamino, acetylamino, and the like), and the like. Prodrugs of compounds of Formula (I) are also within the scope of this invention.

The present invention also includes protected derivatives of compounds of Formula (I). For example, when compounds of Formula (I) contain groups such as hydroxy, carboxy, thiol or any group containing a nitrogen atom(s), these groups can be protected with a suitable protecting groups. A comprehensive list of suitable protective groups can be found in T. W. Greene, Protective Groups in Organic Synthesis, John Wiley & Sons, Inc. (1999), the disclosure of which is incorporated herein by reference in its entirety. The protected derivatives of compounds of Formula (I) can be prepared by methods well known in the art.

A “pharmaceutically acceptable salt” of a compound means a salt that is pharmaceutically acceptable and that possesses the desired pharmacological activity of the parent compound. Such salts include, for example, acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

A “pharmaceutically acceptable salt” can include, for example, salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

The compounds of the present invention may have asymmetric centers. Compounds of the present invention containing an asymmetrically substituted atom may be isolated in optically active or racemic forms. It is well known in the art how to prepare optically active forms, such as by resolution of materials. All chiral, diastereomeric, racemic forms are within the scope of this invention, unless the specific stereochemistry or isomeric form is specifically indicated.

Certain compounds of Formula (I) can exist as tautomers and/or geometric isomers. All possible tautomers and cis and trans isomers, as individual forms and mixtures thereof are within the scope of this invention. Additionally, as used herein the term alkyl includes all the possible isomeric forms of said alkyl group albeit only a few examples are set forth. Furthermore, when the cyclic groups such as aryl, heteroaryl, heterocyclyl are substituted, they include all the positional isomers albeit only a few examples are set forth. Furthermore, all polymorphic forms and hydrates of a compound of Formula (I) are within the scope of this invention.

“Oxo” means ═(O) group.

“Optional” or “optionally” means that the subsequently described event or circumstance may but need not occur, and that the description includes instances where the event or circumstance occurs and instances in which it does not. For example, “heterocyclyl group optionally mono- or di-substituted with an alkyl group” means that the alkyl may but need not be present, and the description includes situations where the heterocyclyl group is mono- or disubstituted with an alkyl group and situations where the heterocyclyl group is not substituted with the alkyl group.

“Optionally substituted phenyl” means a phenyl ring optionally substituted with one, two, or three substituents independently selected from alkyl, halo, alkoxy, alkylthio, haloalkyl, haloalkoxy, amino, alkylamino, dialkylamino, hydroxy, cyano, nitro, aminocarbonyl, acylamino, sulfonyl, hydroxyalkyl, alkoxycarbonyl, aminoalkyl, alkoxycarbonyl, carboxy, cycloalkyl, cycloalkylalkyl, cycloalkoxy, cycloalkylalkyloxy, sulfinyl, and sulfonyl, each as defined herein.

“Optionally substituted heteroaryl” means a monovalent monocyclic or bicyclic aromatic radical of 5 to 10 ring atoms where one or more, preferably one, two, or three, ring atoms are heteroatoms independently selected from N, O, and S, the remaining ring atoms being carbon that is optionally substituted with one, two, or three substituents independently selected from alkyl, halo, alkoxy, alkylthio, haloalkyl, haloalkoxy, amino, alkylamino, dialkylamino, hydroxy, cyano, nitro, aminocarbonyl, acylamino, sulfonyl, hydroxyalkyl, alkoxycarbonyl, aminoalkyl, alkoxycarbonyl, carboxy, carboxy, cycloalkyl, cycloalkylalkyl, cycloalkoxy, cycloalkylalkyloxy, sulfinyl, and sulfonyl, each as defined herein. More specifically the term optionally substituted heteroaryl includes, but is not limited to, optionally substituted pyridyl, pyrrolyl, imidazolyl, thienyl, furanyl, indolyl, quinolyl, pyrazinyl, pyrimidinyl, pyridazinyl, oxazolyl, isoxazolyl, benzoxazolyl, quinolinyl, isoquinolinyl, benzopyranyl, and thiazolyl that can be optionally substituted as defined above.

“Optionally substituted heterocyclyl” means a saturated or unsaturated monovalent cyclic group of 3 to 8 ring atoms in which one or two ring atoms are heteroatoms independently selected from N, O, and S(O)ₙ, where n is an integer from 0 to 2, the remaining ring atoms being C. One or two ring carbon atoms can optionally be replaced by a —CO— group and is optionally substituted with one, two, or three substituents independently selected from alkyl, halo, alkoxy, alkylthio, haloalkyl, haloalkoxy, amino, alkylamino, dialkylamino, hydroxy, cyano, nitro, aminocarbonyl, acylamino, sulfonyl, hydroxyalkyl, alkoxycarbonyl, aminoalkyl, alkoxycarbonyl, carboxy, carboxy, cycloalkyl, cycloalkylalkyl, cycloalkoxy, cycloalkylalkyloxy, sulfinyl, and sulfonyl, each as defined herein.

A “pharmaceutically acceptable carrier or excipient” means a carrier or an excipient that is useful in preparing a pharmaceutical composition that is generally safe, non-toxic and neither biologically nor otherwise undesirable, and includes a carrier or an excipient that is acceptable for veterinary use as well as human pharmaceutical use. “A pharmaceutically acceptable carrier/excipient” as used in the specification and claims includes both one and more than one such excipient.

“Sulfinyl” means a —SOR radical where R is alkyl, haloalkyl, aryl, aralkyl, heteroaryl, heteroaralkyl, heterocyclyl, or heterocyclylalkyl, each as defined above, e.g., methylsulfinyl, phenylsulfinyl, benzylsulfinyl, pyridinylsulfinyl, and the like.

“Sulfonyl” means a —SO₂R radical where R is alkyl, haloalkyl, aryl, aralkyl, heteroaryl, heteroaralkyl, heterocyclyl, or heterocyclylalkyl, each as defined above, e.g., methylsulfonyl, phenylsulfonyl, benzylsulfonyl, pyridinylsulfonyl, and the like.

“Treating” or “treatment” of a disease includes:
(1) preventing the disease, i.e. causing the clinical symptoms of the disease not to develop in a mammal that may be exposed to or predisposed to the disease but does not yet experience or display symptoms of the disease;
(2) inhibiting the disease, i.e., arresting or reducing the development of the disease or its clinical symptoms; or
(3) relieving the disease, i.e., causing regression of the disease or its clinical symptoms.

A “therapeutically effective amount” means the amount of a compound of Formula (I) that, when administered to a mammal for treating a disease, is sufficient to effect such treatment for the disease. The “therapeutically effective amount” will vary depending on the compound, the disease and its severity and the age, weight, etc., of the mammal to be treated.

### EMBODIMENTS

In certain embodiments, a compound of Formula (I) or an individual stereoisomer, a mixture of stereoisomers, or a pharmaceutically acceptable salt thereof, as are described in the Summary of the Invention are provided.

(1) In one embodiment, X and Y are nitrogen and Z is —CH═.

(2) In another embodiment, Y and Z are nitrogen and X is —CH═.

(3) In yet another embodiment, X and Z are nitrogen and Y is ═CH—.

(4) In yet another embodiment, Y and Z are nitrogen and X is —CR═ where R is alkyl.

(5) In another embodiment, Y and Z are nitrogen and X is —CR═ where R is methyl, ethyl, n- or iso-propyl.

(6) In another embodiment, compounds of Formula (I) are provided wherein Y and Z are nitrogen and X is —CR═ where R is halo. Within this embodiment, one group of compounds of Formula (I) is that wherein R is fluoro or chloro.

(A) Within the above embodiments (1)-(6), and subgroups contained therein, one group of compounds of Formula (I) is that wherein R¹ is hydrogen.

(B) Within the above embodiments (1)-(6), and subgroups contained therein, another group of compounds of Formula (I) is that wherein R¹ is hydrogen, R² is alkoxy, and R³ is cycloalkoxy or cycloalkylalkyloxy. Within this embodiment, one group of compounds is that wherein R² is methoxy, and R³ is cyclopropoxy, cyclobutyoxy, cyclopentoxy, or cyclohexyloxy. Within this embodiment, another group of compounds is that wherein R² is methoxy, and R³ is cyclopropylmethyloxy, cyclopropylethoxy, cyclobutylmethyloxy, cyclobutylethyloxy, cyclopentylmethyloxy, cyclohexylmethyloxy or cyclohexylethyloxy.

(C) Within the above embodiments (1)-(6), and subgroups contained therein, yet another group of compounds of Formula (I) is that wherein R¹ is hydrogen, R² is alkoxy, preferably methoxy or ethoxy, and R³ is hydroxyalkyl.

(D) Within the above embodiments (1)-(6), and subgroups contained therein, yet another group of compounds of Formula (I) is that wherein R¹ is hydrogen, R² is alkoxy, preferably methoxy or ethoxy, and R³ is hydroxyalkyloxy.

(E) Within the above embodiments (1)-(6), and subgroups contained therein, yet another group of compounds of Formula (I) is that wherein R¹ is hydrogen, R² is alkoxy, preferably methoxy or ethoxy, and R³ is alkoxyalkyl.

(F) Within the above embodiments (1)-(6), and subgroups contained therein, yet another group of compounds of Formula (I) is that wherein R¹ is hydrogen, R² is alkoxy, preferably methoxy or ethoxy, and R³ is alkoxyalkyloxy.

(G) Within the above embodiments (1)-(6), and subgroups contained therein, yet another group of compounds of Formula (I) is that wherein R¹ is hydrogen, R² is alkoxy, preferably methoxy or ethoxy, and R³ is -(alkylene)NR¹³R¹⁴.

(H) Within the above embodiments (1)-(6), and subgroups contained therein, yet another group of compounds of Formula (I) is that wherein R¹ is hydrogen, R² is alkoxy, preferably methoxy or ethoxy, and R³ is —O-(alkylene)NR¹⁵R¹⁶.

(I) Within the above embodiments (1)-(6), and subgroups contained therein, yet another group of compounds of Formula (I) is that wherein R¹ is hydrogen, R² is monoalkylamino, dialkylamino, fluoro, or trifluoromethoxy, and R³ is cycloalkoxy, cycloalkylalkyloxy, hydroxyalkoxy, alkoxylalkyloxy, or —O(alkylene)NR¹⁵R¹⁶.

(J) Within the above embodiments (1)-(6), and subgroups contained therein, yet another group of compounds of Formula (I) is that wherein R¹ is hydrogen, R² is alkyl, preferably methyl or ethyl, and R³ is as defined in the Summary of Invention.

(i) Within the above embodiments (1)-(6), and embodiments contained therein, e.g., (1)(A-J), (2)(A-J), (3)(A-J), (4)(A-J), (5)(A-J), and (6)(A-J), and groups contained therein, one group of compounds of Formula (I) is that wherein R^{3a} is a ring of formula (a): 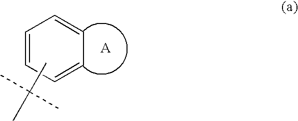
where A is a monocyclic five-, six-, or seven membered heterocyclyl ring substituted with R⁴, R⁵ and R⁶ as defined in the Summary of the Invention.

(ii) Within the above embodiments (1)-(6), and embodiments contained therein, e.g., (1)(A-J), (2)(A-J), (3)(A-J), (4)(A-J), (5)(A-J), and (6)(A-J), another group of compounds of Formula (I) is that wherein R^{3a} is a ring of formula:

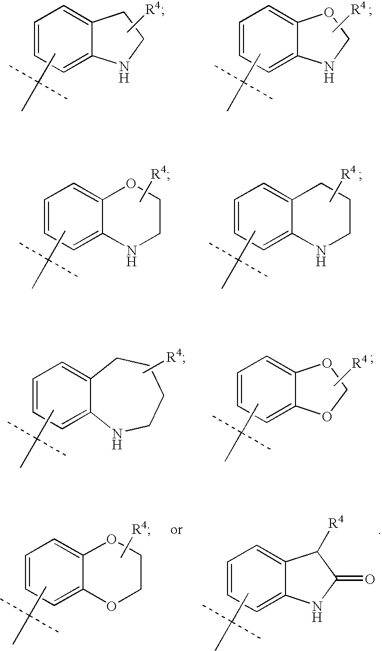

(iii) Within the above embodiments (1)-(6), and embodiments contained therein, e.g., (1)(A-J), (2)(A-J), (3)(A-J), (4)(A-J), (5)(A-J), and (6)(A-J), and groups contained therein, another group of compounds of Formula (I) is that wherein R^{3a} is a ring of formula:

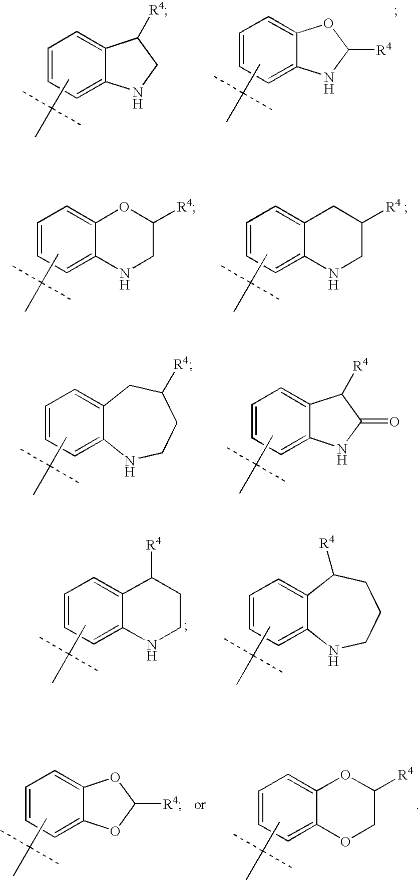

(iv) Within the above embodiments (1)-(6), and embodiments contained therein, e.g., (1)(A-J), (2)(A-J), (3)(A-J), (4)(A-J), (5)(A-J), and (6)(A-J), and groups contained therein, yet another group of compounds of Formula (I) is that wherein R^{3a} is a ring of formula:

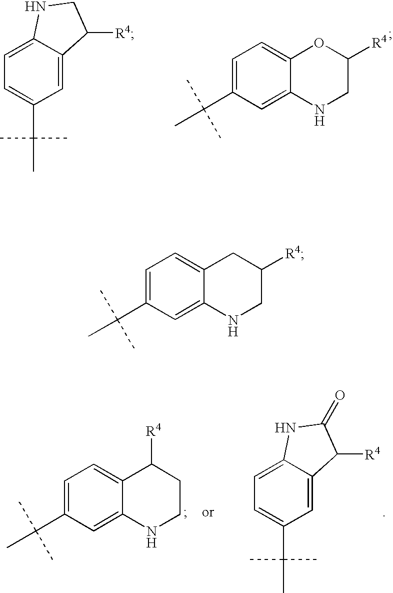

The R⁴ group in (ii)-(iv) is as defined in the Summary of the invention. Within the subgroups (ii)-(iv) above, one group of compounds is that wherein R⁴ is phenyl optionally substituted as defined in the Summary of the Invention.

Within the subgroups (ii)-(iv) above, another group of compounds is that wherein R⁴ is heteroaryl optionally substituted as defined in the Summary of the Invention.

Within the subgroups (ii)-(iv) above, another group of compounds is that wherein R⁴ is a saturated monocyclic heterocyclyl optionally substituted as defined in the Summary of the Invention.

Within the subgroups (ii)-(iv) above, another group of compounds is that wherein R^{3a} is saturated fused heterocyclyl optionally substituted as defined in the Summary of the Invention.

The R^{3a} rings in subgroups (ii)-(iv) above, the subgroups contained therein, including the hydrogen in —NH— groups in the rings, can also be optionally substituted with R⁵ and R⁶ are as defined in the Summary of the Invention. In one embodiment, one of R⁵ and R⁶ is hydrogen.

(v) Within the above embodiments (1)-(6), and embodiments contained therein, e.g., (1)(A-J), (2)(A-J), (3)(A-J), (4)(A-J), (5)(A-J), and (6)(A-J), and groups contained therein, yet another group of compounds of Formula (I) is that wherein R^{3a} is a ring of formula: 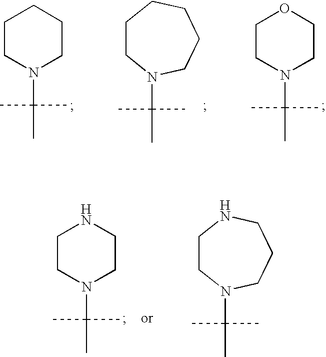
substituted with R⁴, R⁵ and R⁶ as defined in the Summary of the Invention. Within this subgroup, one group of compounds is that wherein the above rings are substituted with R⁴ as defined in the Summary of the Invention and optionally substituted with R⁵ and R⁶ where one of R⁵ and R⁶ is hydrogen. In one group of compounds, the —NH— groups in the rings are substituted with alkyl, cycloalkyl, or cycloalkylalkyl. In another group of compounds, the —NH— groups in the rings are unsubstituted. Within this embodiment, one group of compounds is that wherein R^{3a} is morpholin-1-yl, piperazin-1-yl or homopiperazin-1-yl substituted as defined in (v) above. Within this embodiment, another group of compounds is that wherein R^{3a} is piperidin-1-yl or homopiperidin-1-yl substituted as defined in (v) above.

(vi) Within the above embodiments (1)-(6), and embodiments contained therein, e.g., (1)(A-J), (2)(A-J), (3)(A-J), (4)(A-J), (5)(A-J), and (6)(A-J), and groups contained therein, yet another group of compounds of Formula (I) is that wherein R^{3a} is a ring of formula: 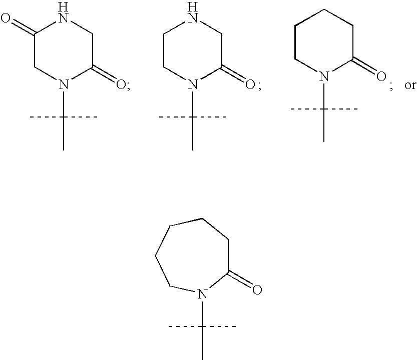
substituted with R⁴, R⁵ and R⁶ as defined in the Summary of the Invention. Within this subgroup, one group of compounds is that wherein the above rings are substituted with R⁴ as defined in the Summary of the Invention, preferably aryl, heteroaryl, or six membered saturated heterocyclyl optionally substituted with R^{a}, R^{b} and R^{c} and substituted with R⁵ and R⁶ where at least one of R⁵ and R⁶ is hydrogen. In one group of compounds, the —NH— groups in the rings are substituted with alkyl, cycloalkyl, or cycloalkylalkyl. In another group of compounds, the —NH— groups in the rings are unsubstituted.

(vii) Within the above embodiments (1)-(6), and embodiments contained therein, e.g., (1)(A-J), (2)(A-J), (3)(A-J), (4)(A-J), (5)(A-J), and (6)(A-J), and groups contained therein, yet another group of compounds of Formula (I) is that wherein R^{3a} is a ring of formula: 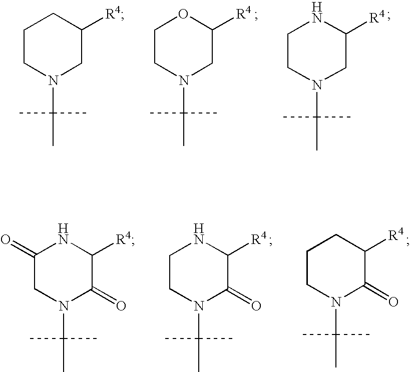
where R⁴ is as defined in the Summary of the Invention. Within this embodiment, one group of compounds is that wherein R⁴ is cycloalkyl, phenyl, heteroaryl, or six membered saturated heterocyclyl optionally substituted with R^{a}, R^{b} and R^{c} and the rings are optionally substituted, including the hydrogen atom on the —NH— group within the ring with R⁵ and R⁶ as defined in the Summary of the Invention, preferably, R⁵ is hydrogen and R⁶ is attached to the carbon adjacent to the nitrogen attached to the cinnoline, quinazoline or phthalazine ring.

(viii) Within the above embodiments (1)-(6), and embodiments contained therein, e.g., (1)(A-J), (2)(A-J), (3)(A-J), (4)(A-J), (5)(A-J), and (6)(A-J), and groups contained therein, yet another group of compounds of Formula (I) is that wherein R^{3a} is a ring of formula: 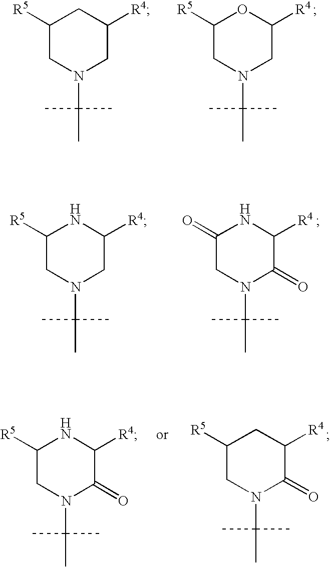
where R⁴ is phenyl or heteroaryl substituted at the para position with R^{a} and optionally substituted with R^{b} and R^{c} wherein R^{a}, R^{b}, and R^{c} are as defined in the Summary of the Invention and R⁵ is as defined in the Summary of the Invention. The —NH— groups in the above rings can optionally be substituted with R⁶ as defined in the Summary of the Invention. In one group of compounds within this embodiment, R⁶ is cycloalkyl, alkyl, or cycloalkylalkyl. In one group of compounds within this embodiment R^{3a} is other than piperidin-1-yl substituted as described above. In one group of compounds within this embodiment R^{3a} is piperidin-1-yl substituted as described above. In another group of compounds within this embodiment, R⁴ is phenyl substituted with R^{a} and R^{b} where R^{a} and R^{b} are meta to each other. Within this embodiment, yet another group of compounds is that wherein R⁴ is —NHCOR⁷ where R⁷ is aryl or heteroaryl as defined in the Summary of the Invention.

(ix) Within the above embodiments (1)-(6), and embodiments contained therein, e.g., (1)(A-J), (2)(A-J), (3)(A-J), (4)(A-J), (5)(A-J), and (6)(A-J), and groups contained therein, yet another group of compounds of Formula (I) is that wherein R^{3a} is a ring of formula: 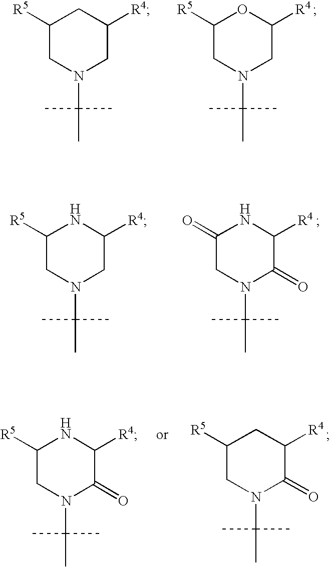
where R⁴ is heterocyclyl, preferably heterocyclyl containing at least a —C═O group wherein the heterocyclyl ring is optionally substituted at the para position with R^{a} and optionally substituted with R^{b} and R^{c} wherein R^{a}, R^{b}, and R^{c} are as defined in the Summary of the Invention and R⁵ is as defined in the Summary of the Invention. Within this group, in one embodiment, R⁴ is monocyclic saturated six membered ring containing at least a —C═O group and optionally substituted at the para position with R^{a} and optionally substituted with R^{b} and R^{c} wherein R^{a}, R^{b}, and R^{c} are as defined in the Summary of the Invention. The —NH— groups in the above rings can optionally be substituted with R⁶ as defined in the Summary of the Invention. Preferably, R⁶ is cycloalkyl, alkyl, or cycloalkylalkyl. In one group of compounds within this embodiment R^{3a} is other than piperidin-1-yl substituted as described above. In one group of compounds within this embodiment R^{3a} is piperidin-1-yl substituted as described above.

(x) Within the above embodiments (1)-(6), and embodiments contained therein, e.g., (1)(A-J), (2)(A-J), (3)(A-J), (4)(A-J), (5)(A-J), and (6)(A-J), and groups contained therein, yet another group of compounds of Formula (I) is that wherein R^{3a} is a ring of formula: 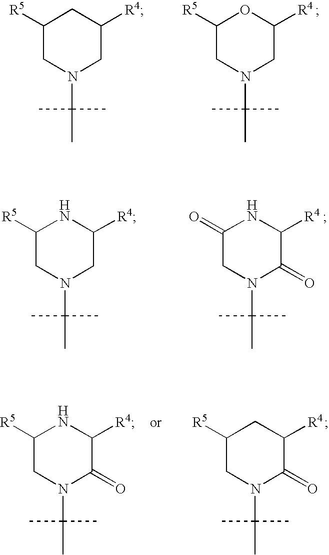
where R⁴ is cycloalkyl substituted at the para position with R^{a} and optionally substituted with R^{b} and R^{c} wherein R^{a}, R^{b}, and R^{c} are as defined in the Summary of the Invention and R⁵ is as defined in the Summary of the Invention. The —NH— groups in the above rings can optionally be substituted with R⁶ as defined in the Summary of the Invention. In one group of compounds within this embodiment R⁶ is cycloalkyl, alkyl, or cycloalkylalkyl.

(xi) Within the above embodiments (1)-(6), and embodiments contained therein, e.g., (1)(A-J), (2)(A-J), (3)(A-J), (4)(A-J), (5)(A-J), and (6)(A-J), and groups contained therein, yet another group of compounds of Formula (I) is that wherein R^{3a} is a ring of formula: 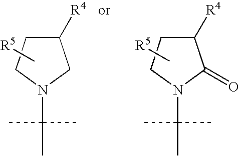
where R⁴ and R⁵ are as defined in the Summary of the Invention.

(xii) Within the above embodiments (1)-(6), and embodiments contained therein, e.g., (1)(A-J), (2)(A-J), (3)(A-J), (4)(A-J), (5)(A-J), and (6)(A-J), and groups contained therein, yet another group of compounds of Formula (I) is that wherein R^{3a} is a ring of formula (b). In one group of compounds is that wherein R^{3a} is a ring of formula: 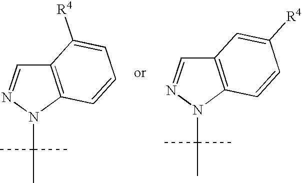
where R⁴ is cycloalkyl, aryl, heteroaryl, heterocyclyl, aralkyl, heteroaralkyl, heterocyclylalkyl, or —X¹R⁷ (where X¹ is —O—, —CO—, —NR⁸CO—, —CONR⁹—, —NR¹⁰—, —S—, —SO—, —SO₂—, —NR¹¹SO₂—, or —SO₂NR¹²— where R⁸, R⁹, R¹⁰, R¹¹ and R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl); more preferably phenyl, heteroaryl or heterocyclyl; and optionally substituted with R⁵ and R⁶ are independently hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, or disubstituted amino; and wherein the aromatic or alicyclic ring in R⁴, R⁵, R⁶, and R⁷ is optionally substituted with one to three substitutents independently selected from R^{a}, R^{b}, and R^{c} which are alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, or disubstituted amino; and additionally substituted with one or two substitutents independently selected from R^{d} and R^{e} where R^{d} and R^{e} are hydrogen or fluoro.

Within this embodiment, one group of compounds is that wherein R^{3a} is: 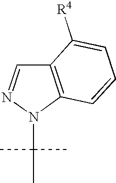
where R⁴ is phenyl, heteroaryl or five or six membered heterocyclyl optionally substituted with one to three substitutents independently selected from R^{f}, R^{g}, and R^{h} as defined in the Summary of the Invention.

Within this embodiment, one group of compounds is that wherein R^{3a} is: 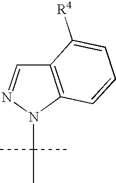
where R⁴ is morpholin-4-yl, piperazin-1-yl, or pyridinyl optionally substituted with one to three substitutents independently selected from R^{f}, R^{g}, and R^{h} as defined in the Summary of the Invention.

(xiii) Within the above embodiments (1)-(6), and embodiments contained therein, e.g., (1)(A-J), (2)(A-J), (3)(A-J), (4)(A-J), (5)(A-J), and (6)(A-J), and groups contained therein, yet another group of compounds of Formula (I) is that wherein R^{3a} is a ring of formula: 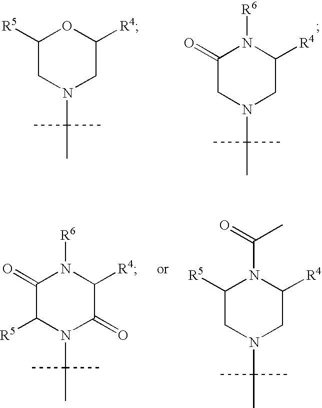
where R⁴ is cyclopentyl, cyclohexyl, phenyl, heteroaryl, or monocyclic saturated five or six membered heterocyclyl ring; R⁵ is hydrogen, alkyl, phenyl, heteroaryl, or monocyclic five or six membered heterocyclyl ring; and R⁶ is alkyl, preferably methyl; and wherein the aromatic or alicyclic ring in R⁴ and R⁵ are optionally substituted with R^{a}, R^{b} and R^{c} as defined in the Summary of the Invention. Within this subgroup, in one embodiment, R⁴ is phenyl, heteroaryl, or monocyclic five or six membered heterocyclyl ring and R⁵ is hydrogen or alkyl. In another embodiment, R⁴ and R⁵ are independently phenyl, heteroaryl, or monocyclic saturated five or six membered heterocyclyl ring. In each of the above embodiments, the aromatic or alicyclic ring are optionally substituted with R^{a} selected from alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl and R^{b} and R^{c} independently selected from alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, or disubstituted amino.

(xiv) Within the above embodiments (1)-(6), and embodiments contained therein, e.g., (1)(A-J), (2)(A-J), (3)(A-J), (4)(A-J), (5)(A-J), and (6)(A-J), and groups contained therein, yet another group of compounds of Formula (I) is that wherein R^{3a} is a ring of formula: 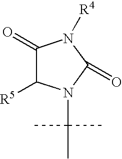
where R⁴ is aralkyl, preferably benzyl optionally substituted with R^{a}, R^{b} and R^{c} as defined in the Summary of the Invention and R⁵ is as defined in the Summary of the Invention, preferably hydrogen or alkyl.

(xv) Within the above embodiments (1)-(6), and embodiments contained therein, e.g., (1)(A-J), (2)(A-J), (3)(A-J), (4)(A-J), (5)(A-J), and (6)(A-J), and groups contained therein, yet another group of compounds of Formula (I) is that wherein R^{3a} is a ring of formula (a): 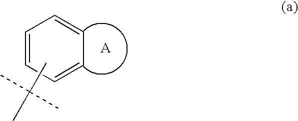
where A is a monocyclic five-, six-, or seven membered heterocyclyl ring and the ring (a) is substituted with R⁴, R⁵ and R⁶ as defined below.

R⁴ is cycloalkyl, cycloalkylalkyl, aryl, heteroaryl, heterocyclyl, aralkyl, heteroaralkyl, heterocyclylalkyl, or —X¹R⁷ (where X¹ is —O—, —CO—, —NR⁸CO—, —CONR⁹—, —NR¹⁰—, —S—, —SO—, —SO₂—, —NR¹¹SO₂—, or —SO₂NR¹²— where R⁸, R⁹, R¹⁰, R¹¹ and R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl).

R⁵ is hydrogen alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl.

R⁶ is hydrogen, alkyl, alkoxy, halo, haloalkyl, haloalkoxy, hydroxyl, cyano, nitro, carboxy, alkoxycarbonyl, alkylthio, sulfinyl, sulfonyl, acyl, aminocarbonyl, aminosulfinyl, aminosulfonyl, or monosubstituted amino, disubstituted amino, preferably hydrogen.

The aromatic or alicyclic ring in R⁴, R⁵, R⁶, and R⁷ is optionally substituted with one to three substitutents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substitutents independently selected from R^{d} and R^{e} where R^{d} and R^{e} are hydrogen or fluoro.

In one embodiment, A is a saturated five or six membered heterocyclyl ring and substituted as described above.

(xvi) Within the above embodiments (1)-(6), and embodiments contained therein, e.g., (1)(A-J), (2)(A-J), (3)(A-J), (4)(A-J), (5)(A-J), and (6)(A-J), and groups contained therein, yet another group of compounds of Formula (I) is that wherein R^{3a} is a ring of formula (b): 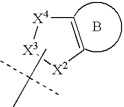
where X², X³, and X⁴ are independently carbon, nitrogen, oxygen or sulfur provided that at least two of X², X³, and X⁴ is other than carbon; and B is phenyl, or a six-membered heteroaryl ring (wherein the six-membered heteroaryl ring contains one or two nitrogen atoms, the rest of the ring atoms being carbon), or a monocyclic five-, six-, or seven-membered heterocyclyl ring; and wherein formula (b) is substituted with R⁴, R⁵ and R⁶ as defined below.

R⁴ is cycloalkyl, cycloalkylalkyl, aryl, heteroaryl, heterocyclyl, aralkyl, heteroaralkyl, heterocyclylalkyl, or —X¹R⁷ (where X¹ is —O—, —CO—, —NR⁸CO—, —CONR⁹—, —NR¹⁰—, —S—, —SO—, —SO₂—, —NR¹¹SO₂—, or —SO₂NR¹²— where R⁸, R⁹, R¹⁰, R¹¹ and R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl).

R⁵ is hydrogen alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl.

R⁶ is hydrogen, alkyl, alkoxy, halo, haloalkyl, haloalkoxy, hydroxyl, cyano, nitro, carboxy, alkoxycarbonyl, alkylthio, sulfinyl, sulfonyl, acyl, aminocarbonyl, aminosulfinyl, aminosulfonyl, or monosubstituted amino, disubstituted amino, preferably hydrogen; and

The aromatic or alicyclic ring in R⁴, R⁵, R⁶, and R⁷ is optionally substituted with one to three substitutents independently selected from R^{a}, R^{b}, and R^{e} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substitutents independently selected from R^{d} and R^{e} where R^{d} and R^{e} are hydrogen or fluoro.

(xvii) Within the above embodiments (1)-(6), and embodiments contained therein, e.g., (1)(A-J), (2)(A-J), (3)(A-J), (4)(A-J), (5)(A-J), and (6)(A-J), and groups contained therein, yet another group of compounds of Formula (I) is that wherein R^{3a} is a monocyclic six- or seven-membered heterocyclyl ring substituted with R⁴, R⁵ and R⁶ as defined below.

R⁴ is selected from aryl, heteroaryl, heterocyclyl, aralkyl, heteroaralkyl, heterocyclylalkyl, or —X¹R⁷ (where X¹ is —O—, —CO—, —NR⁸CO—, —CONR⁹—, —NR¹⁰—, —S—, —SO—, —SO₂—, —NR¹¹SO₂—, or —SO₂NR¹²— where R⁸, R⁹, R¹⁰, R¹¹ and R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl).

R⁵ is alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl.

R⁶ is hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, or disubstituted amino, preferably hydrogen.

The aromatic or alicyclic ring in R⁴, R⁵, R⁶, and R⁷ is optionally substituted with one to three substitutents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substitutents independently selected from R^{d} and R^{e} where R^{d} and R^{e} are hydrogen or fluoro.

In one group within this embodiment, R^{3a} is other than piperidinyl substituted as described above.

In one group within this embodiment, R^{3a} is piperidinyl substituted as described above.

(xviii) Within the above embodiments (1)-(6), and embodiments contained therein, e.g., (1)(A-J), (2)(A-J), (3)(A-J), (4)(A-J), (5)(A-J), and (6)(A-J), and groups contained therein, yet another group of compounds of Formula (I) is that wherein R^{3a} is pyrrolidin-1-yl substituted with R⁴, R⁵ and R⁶ as defined below.

R⁴ is cycloalkyl, cycloalkylalkyl, aryl, heteroaryl, heterocyclyl, aralkyl, heteroaralkyl, heterocyclylalkyl, or —X¹R⁷ (where X¹ is —O—, —CO—, —NR⁸CO—, —CONR⁹—, —NR¹⁰—, —S—, —SO—, —SO₂—, —NR¹¹SO₂—, or —SO₂NR¹²— where R⁸, R⁹, R¹⁰, R¹¹ and R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl).

R⁵ is hydrogen alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl.

R⁶ is hydrogen, alkyl, alkoxy, halo, haloalkyl, haloalkoxy, hydroxyl, cyano, nitro, carboxy, alkoxycarbonyl, alkylthio, sulfinyl, sulfonyl, acyl, aminocarbonyl, aminosulfinyl, aminosulfonyl, or monosubstituted amino, disubstituted amino, preferably hydrogen.

The aromatic or alicyclic ring in R⁴, R⁵, R⁶, and R⁷ is optionally substituted with one to three substitutents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substitutents independently selected from R^{d} and R^{e} where R^{d} and R^{e} are hydrogen or fluoro.

(xix) Within the above embodiments (1)-(6), and embodiments contained therein, e.g., (1)(A-J), (2)(A-J), (3)(A-J), (4)(A-J), (5)(A-J), and (6)(A-J), and groups contained therein, yet another group of compounds of Formula (I) is that wherein R^{3a} is 2-oxopyrrolidinyl or 2,4-dioxoimidazolidinyl substituted with R⁴, R⁵ and R⁶ as defined below.

R⁴ is cycloalkyl, cycloalkylalkyl, aryl, heteroaryl, heterocyclyl, aralkyl, heteroaralkyl, heterocyclylalkyl, or —X¹R⁷ (where X¹ is —O—, —CO—, —NR⁸CO—, —CONR⁹—, —NR¹⁰—, —S—, —SO—, —SO₂—, —NR¹¹SO₂—, or —SO₂NR¹²— where R⁸, R⁹, R¹⁰, R¹¹ and R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl).

R⁵ is hydrogen alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl.

R⁶ is hydrogen, alkyl, alkoxy, halo, haloalkyl, haloalkoxy, hydroxyl, cyano, nitro, carboxy, alkoxycarbonyl, alkylthio, sulfinyl, sulfonyl, acyl, aminocarbonyl, aminosulfinyl, aminosulfonyl, or monosubstituted amino, disubstituted amino, preferably hydrogen.

The aromatic or alicyclic ring in R⁴, R⁵, R⁶, and R⁷ is optionally substituted with one to three substitutents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substitutents independently selected from R^{d} and R^{e} where R^{d} and R^{e} are hydrogen or fluoro.

(xx) Within the above embodiments (1)-(6), and embodiments contained therein, e.g., (1)(A-J), (2)(A-J), (3)(A-J), (4)(A-J), (5)(A-J), and (6)(A-J), and groups contained therein, yet another group of compounds of Formula (I) is that wherein R^{3a} is phenyl optionally substituted as defined in the Summary of the Invention.

Within this embodiment, one group of compounds is that wherein R^{3a} is a group of formula: 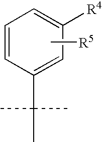
where one of R⁴ and R⁵ is hydrogen, alkyl, halo, haloalkyl, alkoxy, haloalkoxy, cyano, amino, monosubstituted or disubstituted amino, or —X¹R⁷ (where X¹ is —O—, —CO—, —NR⁸CO—, —CONR⁹—, —S—, —SO—, —SO₂—, —NR¹¹SO₂—, or —SO₂NR¹²— where R⁸, R⁹, R¹⁰, R¹¹ and R¹² are independently hydrogen, alkyl, hydroxyalkyl, alkoxyalkyl, aryl, aralkyl, heteroaryl, heteroaralkyl, acyl, or heterocyclylalkyl and R⁷ is alkyl, alkoxyalkyl, hydroxyalkyl, aminoalkyl, cycloalkyl, cycloalkylalkyl, aryl, heteroaryl, heterocyclyl, aralkyl, heteroaralkyl, or heterocyclylalkyl); and the other of R⁴ and R⁵ is cycloalkyl, aryl, heteroaryl, or heterocyclyl; and wherein the aromatic or alicyclic ring in R⁴ and R⁵ is optionally substituted with one to three substitutents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, or optionally substituted heterocyclyl. Preferably, R⁴ is aryl, heteroaryl, or heterocyclyl optionally substituted with one to three substitutents independently selected from R^{a}, R^{b}, and R^{c}.

(xxi) Within the above embodiments (1)-(6), and embodiments contained therein, e.g., (1)(A-J), (2)(A-J), (3)(A-J), (4)(A-J), (5)(A-J), and (6)(A-J), and groups contained therein, yet another group of compounds of Formula (I) is that wherein R³, is a group of formula: 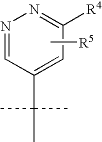
where R⁴ and R⁵ are as defined in (xvii) above.

(xxii) Within the above embodiments (1)-(6), and embodiments contained therein, e.g., (1)(A-J), (2)(A-J), (3)(A-J), (4)(A-J), (5)(A-J), and (6)(A-J), and groups contained therein, yet another group of compounds of Formula (I) is that wherein R^{3a} is a group of formula: 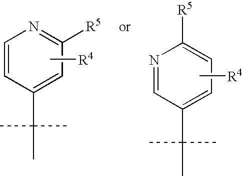
where R⁴ and R⁵ are as defined in (xxi) above.

Within this subgroup (xxii), another class of compounds is that where R⁵ is heteroaryl optionally substituted with one to three substitutents independently selected from R^{a}, R^{b}, and R^{c}. Within this subgroup (xxii), another class of compounds is that where R⁵ is heterocyclyl, preferably piperazinyl, piperidinyl, or morpholinyl, optionally substituted with one to three substitutents independently selected from R^{a}, R^{b}, and R^{c}. Within this subgroup (xxii), another class of compounds is that where R⁵ is mono or disubstituted amino and R⁴ is hydrogen, alkyl, or halo.

(xxiii) Within the above embodiments (1)-(6), and embodiments contained therein, e.g., (1)(A-J), (2)(A-J), (3)(A-J), (4)(A-J), (5)(A-J), and (6)(A-J), and groups contained therein, yet another group of compounds of Formula (I) is that wherein R^{3a} is a group of formula: 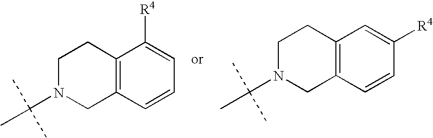
where R⁴ is as defined in the Summary of the Invention. The isoquinoline ring can optionally be substituted with R⁵ as defined in the Summary of the Invention.

Within this subgroup (xxiii), another class of compounds is that where R⁴ is heteroaryl optionally substituted with one to three substitutents independently selected from R^{a}, R^{b}, and R^{c}. Within this subgroup (xxiii), another class of compounds is that where R⁴ is heterocyclyl, preferably piperazinyl, piperidinyl, or morpholinyl, optionally substituted with one to three substitutents independently selected from R^{a}, R^{b} and R^{c}.

(xxiv) Within the above embodiments (1)-(6), and embodiments contained therein, e.g., (1)(A-J), (2)(A-J), (3)(A-J), (4)(A-J), (5)(A-J), and (6)(A-J), and groups contained therein, yet another group of compounds of Formula (I) is that wherein R^{3a} is a group of formula: 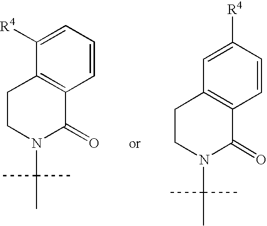
where R⁴ is as defined in the Summary of the Invention. The isoquinoline ring can optionally be substituted with R⁵ as defined in the Summary of the Invention.

Within this subgroup (xxiv), another class of compounds is that where R⁴ is heteroaryl optionally substituted with one to three substitutents independently selected from R^{a}, R^{b}, and R^{c}. Within this subgroup (xxiv), another class of compounds is that where R⁴ is heterocyclyl, preferably piperazinyl, piperidinyl, or morpholinyl, optionally substituted with one to three substitutents independently selected from R^{a}, R^{b}, and R^{c}.

(xxv) Within the above embodiments (1)-(6), and embodiments contained therein, e.g., (1)(A-J), (2)(A-J), (3)(A-J), (4)(A-J), (5)(A-J), and (6)(A-J), and groups contained therein, yet another group of compounds of Formula (I) is that wherein R^{3a} is a group of formula: 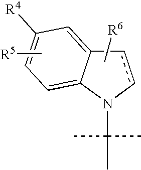
where and R⁴, R⁵ and R⁶ are as defined in the Summary of the Invention.

Within this embodiment, one class of compounds is that wherein R^{3a} is a group of formula: 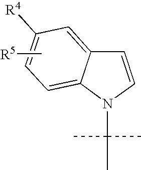
where one of R⁴ and R⁵ is hydrogen, alkyl, halo, haloalkyl, alkoxy, haloalkoxy, cyano, amino, monosubstituted or disubstituted amino, or —X¹R⁷ (where X¹ is —O—, —CO—, —NR⁸CO—, —CONR⁹—, —S—, —SO—, —SO₂—, —NR¹⁰SO₂—, or —SO₂NR¹¹— where R⁸, R⁹, R¹⁰ and R¹¹ are independently hydrogen, alkyl, hydroxyalkyl, alkoxyalkyl, aryl, aralkyl, heteroaryl, heteroaralkyl, acyl, or heterocyclylalkyl and R⁷ is alkyl, alkoxyalkyl, hydroxyalkyl, aminoalkyl, cycloalkyl, cycloalkylalkyl, aryl, heteroaryl, heterocyclyl, aralkyl, heteroaralkyl, or heterocyclylalkyl); and the other one of R⁴ and R⁵ is aryl, heteroaryl, or heterocyclyl; and wherein the aromatic or alicyclic ring in R⁴ and R⁵ is optionally substituted with one to three substitutents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, or optionally substituted heterocyclyl. Preferably, R⁴ is aryl, heteroaryl, or heterocyclyl optionally substituted with one to three substitutents independently selected from R^{a}, R^{b}, and R^{c}.

Within this embodiment, another class of compounds is that of formula: 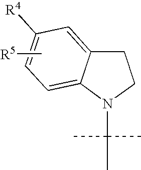
where R⁴ and R⁵ are as described immediately above.

(xxvi) Within the above embodiments (1)-(6), and embodiments contained therein, e.g., (1)(A-J), (2)(A-J), (3)(A-J), (4)(A-J), (5)(A-J), and (6)(A-J), and groups contained therein, another class of compounds is that wherein R^{3a} is a group of formula: 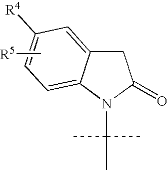
where R⁴ and R⁵ are as described immediately above.

(xxvii) Within the above embodiments (1)-(6), and embodiments contained therein, e.g., (1)(A-J), (2)(A-J), (3)(A-J), (4)(A-J), (5)(A-J), and (6)(A-J), and groups contained therein, yet another group of compounds of Formula (I) is that wherein R^{3a} is a group of formula: 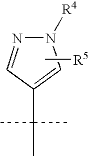
where R⁵ is hydrogen or alkyl and R⁴ is aryl, heteroaryl, aralkyl, heteroaralkyl, or heterocyclyl optionally substituted with one to three substitutents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, or optionally substituted heterocyclyl.

In one embodiment, R⁴ is aralkyl (preferably benzyl) optionally substituted with one to three substitutents independently selected from R^{a}, R^{b}, and R^{c}. In another embodiment, R⁴ is heteroaryl optionally substituted with one to three substitutents independently selected from R^{a}, R^{b}, and R^{c}. In one embodiment, R⁴ is heterocyclyl optionally substituted with optionally substituted phenyl, optionally substituted heteroaryl.

Preferably, R^{3a} is a group of formula: 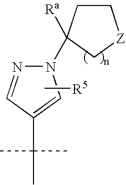
where R⁵ is hydrogen or alkyl, preferably hydrogen; n is 1, 2, or 3; Z is —O—, —NH— or —N-alkyl-; and R^{a} is phenyl or heteroaryl optionally substituted with R^{a}, R^{b}, and R^{c}, preferably phenyl optionally substituted with R^{a}, R^{b}, and R^{c}.

(xxviii) Within the above embodiments (1)-(6), and embodiments contained therein, e.g., (1)(A-J), (2)(A-J), (3)(A-J), (4)(A-J), (5)(A-J), and (6)(A-J), and groups contained therein, and groups contained therein, yet another group of compounds of Formula (I) is that wherein R^{3a} is a group of formula: 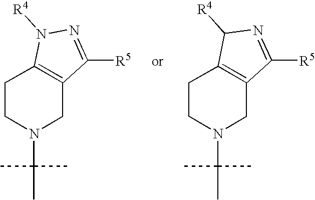
where one of R⁴ and R⁵ is hydrogen, alkyl, halo, haloalkyl, alkoxy, haloalkoxy, cyano, amino, monosubstituted or disubstituted amino, or —X¹R⁷ (where X¹ is —O—, —CO—, —NR⁸CO—, —CONR⁹—, —S—, —SO—, —SO₂—, —NR¹¹SO₂—, or —SO₂NR¹¹— where R⁸, R⁹, R¹⁰ and R¹¹ are independently hydrogen, alkyl, hydroxyalkyl, alkoxyalkyl, aryl, aralkyl, heteroaryl, heteroaralkyl, acyl, or heterocyclylalkyl and R⁷ is alkyl, alkoxyalkyl, hydroxyalkyl, aminoalkyl, cycloalkyl, cycloalkylalkyl, aryl, heteroaryl, heterocyclyl, aralkyl, heteroaralkyl, or heterocyclylalkyl); and the other of R⁴ and R⁵ is aryl, heteroaryl, or heterocyclyl; and wherein the aromatic or alicyclic ring in R⁴ and R⁵ is optionally substituted with one to three substituents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, or optionally substituted heterocyclyl.

Within this embodiment, one group of compounds is that wherein R⁴ is phenyl, heteroaryl, or heterocyclyl optionally substituted with one to three substitutents independently selected from R^{a}, R^{b}, and R^{c}.

(xxix) Within the above embodiments (1)-(6), and embodiments contained therein, e.g., (1)(A-J), (2)(A-J), (3)(A-J), (4)(A-J), (5)(A-J), and (6)(A-J), and groups contained therein, yet another group of compounds of Formula (I) is that wherein R^{3a} is a group of formula: 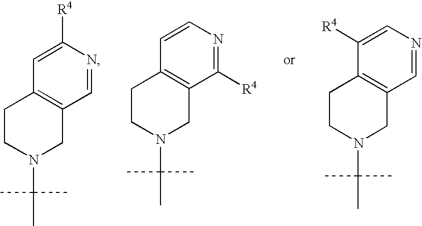
where R⁴ is alkyl, haloalkoxy, cycloalkyl, aryl, heteroaryl, heterocyclyl, or —X¹R⁷ (where X¹ is —O—, —CO—, —NR⁸CO—, —CONR⁹—, —NR¹⁰—, —S—, —SO—, —SO₂—, —NR¹¹SO₂—, or —SO₂NR¹²— where R⁸, R⁹, R¹⁰, R¹¹ and R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl; and wherein the aromatic or alicyclic ring in R⁴ is optionally substituted with one to three substituents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, or optionally substituted heterocyclyl. Preferably, R⁴ is cycloalkyl, aryl, heteroaryl, or heterocyclyl optionally substituted with one to three substituents independently selected from R^{a}, R^{b}, and R^{c}.

(xxx) Within the above embodiments (1)-(6), and embodiments contained therein, e.g., (1)(A-J), (2)(A-J), (3)(A-J), (4)(A-J), (5)(A-J), and (6)(A-J), and groups contained therein, yet another group of compounds of Formula (I) is that wherein R^{3a} is a group of formula: 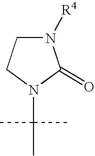
where R⁴ is aralkyl, preferably benzyl optionally substituted with R^{a}, R^{b} and R^{c} as defined in the Summary of the Invention.

Within certain embodiments, a compound as described herein is provided, with the proviso that when X and Z are nitrogen, R¹ is hydrogen, and when one of R² and R³ is hydroxyalkyloxy, alkoxyalkyl, alkoxyalkyloxy or —O-(alkylene)NR¹⁵R¹⁶ (wherein R¹⁵ and R¹⁶ are independently hydrogen or alkyl) and the other of R² and R³ is hydrogen, alkyl, alkoxy, halo, hydroxyalkyloxy, alkoxyalkyloxy, or —O-(alkylene)NR¹⁹R²⁰ (wherein R¹⁹ and R²⁰ are independently hydrogen or alkyl) then R^{3a} is not
2,3-dihydroindolyl, 2-oxoindolyl, indolyl, 7-aza-2-oxo-indol-3-yl, 4-aza-2-oxo-indol-3-yl, 5,7-diazaoxindol-3-yl, or piperidinyl, each of which is substituted with R⁴, R⁵ or R⁶ as defined above;
6-chloro-7-aza-2-oxo-indol-3-yl; 2-alkyl-5H-pyrrolo[2,3-d]pyrimidin-6(7H)-one-5-yl; 4-carboxypiperidin-1-yl; or
piperazin-1-yl substituted with R⁴, R⁵ or R⁶ at the 4-position of the piperazin-1-yl ring where R⁴, R⁵ or R⁶ are as defined above or where R⁴, R⁵ or R⁶ are hydrogen, alkoxycarbonyl, or —CONHR where R is phenyl substituted with alkoxy, cyano, alkyl, 5-hydroxyindol-1-yl, or cyclopropyl.

Representative compounds of Formula (I) where R³ is hydrogen and other groups are as provided in Table 1 below:

TABLE 1

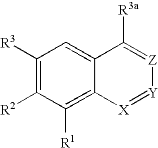

| Cpd # | X | Y | Z | R¹ | R² | R³ | R^{3a} |
|---|---|---|---|---|---|---|---|
| 1 | N | N | CH | H | methoxy | 2-methoxy- | 2-(4-methoxy- |
| | | | | | | ethoxy | phenyl)- |
| | | | | | | | morpholin-4-yl |
| 2 | N | N | CH | H | methoxy | 2-methoxy- | 2-morpholin-4- |
| | | | | | | ethoxy | ylpyridin-5-yl |

General Synthetic Schemes

Compounds of this invention can be made by the methods depicted in the reaction schemes shown below.

The starting materials and reagents used in preparing these compounds are either available from commercial suppliers such as Aldrich Chemical Co., (Milwaukee, Wis.), Bachem (Torrance, Calif.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4th Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure. The starting materials and the intermediates of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure over a temperature range from about −78° C. to about 150° C., more preferably from about 0° C. to about 125° C. and most preferably at about room (or ambient) temperature, e.g., about 20° C.

Compounds of Formula (I) where X and Y are nitrogen, Z is carbon, and R¹, R², R³ and R^{3a} are as defined in the Summary of the Invention can be prepared as described in Scheme 1 below.

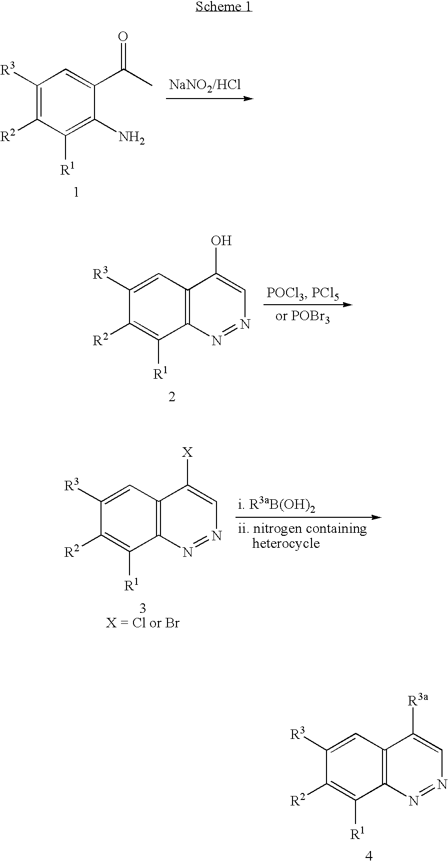

Treatment of 2-aminoacetophenone 1 with sodium nitrite in concentrated hydrochloric acid and water provides diazo compound intermediates that cyclize upon heating to provide 4-hydroxycinnolines 2. Treatment of 2 with either phosphorous oxychloride or phosphorous oxybromide provides the corresponding chloro or bromo compound of formula 3. The chloro derivative 3 can also be prepared by heating 2 in neat phosphorous oxychloride, followed by recrystallization of the product after neutralization (see, for example, Castle et al., J. Org. Chem. 17:1571, 1952). The bromo derivative 3 can also be prepared by mixing a concentrated suspension of the 4-hydroxycinnoline 2 in chloroform and phosphorous oxybromide at room temperature and then warming to reflux for 8 to 16 h. Extractive workup after neutralization and subsequent recrystallization from alcoholic solvent such as ethanol provides 4-bromocinnoline. Alternatively, X can be other suitable leaving groups such as triflate, mesylate, tosylate, and the like that can be prepared by reacting 2 with trifluoromethansulfonyl chloride, mesyl chloride, and tosyl chloride respectively, under conditions well known in the art.

Compounds of formula 1 are either commercially available or can be synthesized by methods well known in the art. For example, compounds of formula 1 wherein R³ is alkyl and R² is cycloalkyloxy, cycloalkylalkyloxy, hydroxyalkyloxy, alkoxyalkyloxy, or —O-(alkylene)NR¹⁵R¹⁶ wherein R¹³, R¹⁴, R¹⁵, and R¹⁶ are independently hydrogen or alkyl), and wherein one or two carbon atoms in the alkyl chain in hydroxyalkyloxy, alkoxyalkyloxy, or —O-(alkylene)NR⁵R¹⁶ are optionally replaced by one to two oxygen or nitrogen atom(s), can be prepared by the method shown in Scheme 2 below, which exemplifies the synthesis of 1-(2-amino-5-ethyl-4-cyclpropyloxyphenyl)ethanone.

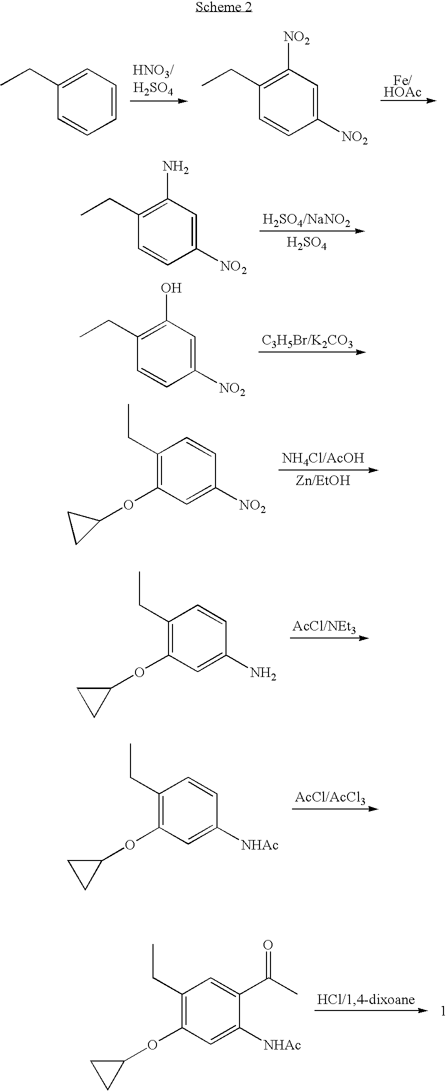

It will be apparent to a person skilled in the art that cyclopropyl bromide can be substituted with other suitable R²LG where R² is as indicated above and LG is a suitable leaving group to give other desired compounds of formula 1.

Compounds of formula 1, wherein R³ is cycloalkyloxy, cycloalkylalkyloxy, hydroxyalkyloxy, alkoxyalkyloxy, or —O-(alkylene)NR⁵R⁶, wherein R³, R⁴, R¹⁵, and R¹⁶ are independently hydrogen or alkyl), and wherein one or two carbon atoms in the alkyl in hydroxyalkyloxy, alkoxyalkyloxy, or —O-(alkylene)NR¹⁵R¹⁶ are optionally replaced by one to two oxygen or nitrogen atom(s), and R² is alkyl may be prepared as shown in Scheme 3 below, which exemplifies the synthesis of 1-(2-amino-4-ethyl-5-cyclopropyloxyphenyl)ethanone.

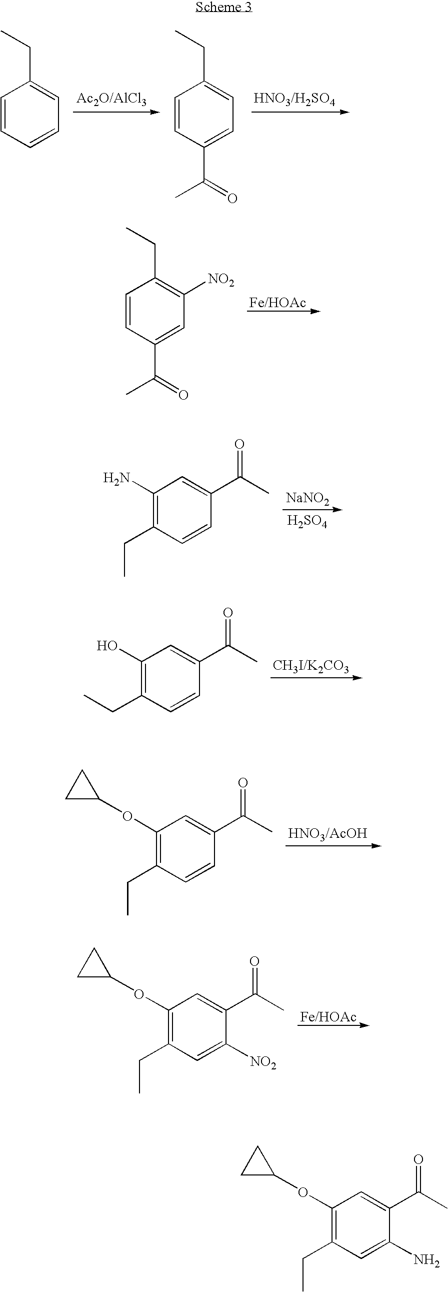

It will be apparent to a person skilled in the art that cyclopropyl bromide can be substituted with other suitable R³LG where R³ is as indicated above and LG is a suitable leaving group to give other desired compounds of formula 1.

Compounds of formula 1 where R¹ is hydrogen and R² and R³ are the same and are selected from cycloalkyloxy, cycloalkylalkyloxy, hydroxyalkyloxy, alkoxyalkyloxy, or —O-(alkylene)NR¹⁵R¹⁶, where R¹³, R¹⁴, R¹⁵, and R¹⁶ are independently hydrogen or alkyl, and wherein one or two carbon atoms in the alkyl chain in hydroxyalkyloxy, alkoxyalkyloxy, or —O-(alkylene)NR¹⁵R¹⁶ are optionally replaced by one to two oxygen or nitrogen atom(s), can be synthesized by methods common to the art. For example, 3,4-dihydroxy-acetophenone can be treated with the desired R³LG where R³ is as defined above and LG is a suitable leaving group in the presence of a base such as cesium carbonate, triethylamine, sodium hydride, potassium carbonate, potassium hydride, and the like to provide the dialkylated product. Suitable organic solvents include acetone, acetonitrile, DMF, THF, and the like. 2-Amino-4,5-disubstituted acetophenones 1 is then prepared by nitration of 4,5-disubstituted acetophenones obtained from above with nitric acid in one of several solvents including acetic acid or sulfuric acid at ice bath temperatures to provide the corresponding 2-nitro4,5-disubstituted acetophenones (Iwamura et al., Bioorg. Med. Chem. 10:675, 2002), followed by reduction of the nitro group under known reaction conditions, e.g., hydrogenation with palladium on carbon, iron powder in acetic acid, or nickel boride (see., Castle et al., J. Org. Chem. 19:1117, 1954).

Compounds of formula 1 where R¹ is hydrogen, R² is cycloalkyloxy, cycloalkylalkyloxy, hydroxyalkyloxy, alkoxyalkyloxy, or —O-(alkylene)NR⁵R⁶, wherein R¹³, R¹⁴, R¹⁵, and R¹⁶ are independently hydrogen or alkyl, and wherein one or two carbon atoms in the alkyl chain in hydroxyalkyloxy, alkoxyalkyloxy, or —O-(alkylene)NR¹⁵R¹⁶ are optionally replaced by one to two oxygen or nitrogen atom(s), and R³ is methoxy can be prepared from acetovanillone (3-methoxy-4-hydroxyacetophenone) as a starting material. Simple etherification, as described above, can be utilized to provide the required 4-substitution, followed by nitration and reduction steps as described above. Alternatively, compounds of formula 1 can be prepared under Mitsunobu reaction conditions by treating phenol with diethyl or diisopropyl azo-dicarboxylates, triphenylphosphine, and the desired alkyl alcohol in THF solution to give the corresponding alkoxy derivative. Compounds of formula 1 where R² and R³ is haloalkoxy can be prepared by treatment of the phenol with haloacetic acid, e.g., chlorodifluoroacetic acid under basic conditions provides difluoromethyl ether.

When compounds of formula 1 where R² and R³ are not the same and are independently cycloalkyloxy, cycloalkylalkyloxy, hydroxyalkyloxy, alkoxyalkyloxy, or —O-(alkylene)NR¹⁵R⁶, wherein R³, R⁴, R¹⁵, and R¹⁶ are independently hydrogen or alkyl, and wherein one or two carbon atoms in the alkyl chain in hydroxyalkyloxy, alkoxyalkyloxy, or —O-(alkylene)NR¹⁵R¹⁶ are optionally replaced by one to two oxygen or nitrogen atom(s), are desired, 3,4-dihydroxyacetophenone can be utilized as the starting material. 3,4-Dihydroxyacetophenone can be selectively protected as its 4-benzyl ether (see Greenspan et al., J. Med. Chem. 42:164, 1999) by treatment with benzyl bromide and lithium carbonate in DMF solution. Functionalization of the 3-OH group with the desired R³LG where R³ and LG are as defined above can be accomplished under the alkylation conditions described above, including Mitsunobu reaction. Removal of the benzyl ether by hydrogenolysis with palladium on carbon in alcoholic solvents such as methanol, ethanol, and the like, and followed by alkylation of the 4-OH with the desired R²LG group would provide the desired 3,4-disubstituted acetophenones. Nitration of the 3,4-disubstituted acetophenones, followed by reduction of the nitro group provides the desired compound 1.

Compounds of formula 3 can be converted to the corresponding compound of Formula (I) via a variety of methods. For example, compounds of Formula (I) wherein R^{3a} is an aryl or heteroaryl ring can be prepared by standard synthetic methods known to one of ordinary skill in the art, for example, by Suzuki-type coupling of the corresponding aryl or heteroaryl boronic acid with compound 3 where X is halo (see, e.g., Miyaura and Suzuki, Chem. Rev. 95:2457-2483, 1995). Such boronic acids are either commercially available (e.g., Aldrich Chemical Co. (Milwaukee, Wis.), Lancaster Synthesis (Ward Hill, Mass.), or Maybridge (Conrwall, UK)) or can readily be prepared from the corresponding bromides by methods described in the literature (see, for example, Miyaura et al, Tetrahedron Letters 1979, 3437; Miyaura and Suzuki, Chem. Commun. 1979, 866).

Compounds of Formula (I) where R^{3a} is heterocyclic ring (e.g., pyrrolidin-1-yl, piperidin-1-yl, morpolin-4-yl, and the like) which are attached to the core ring via a nitrogen atom can be prepared by reacting the 3 (where X is halo or other suitable leaving group such as tosylate, triflate, mesylate and the like) with the heterocyclic ring in the presence of a base such as triethylamine, pyridine. Suitable solvents include, and the not limited to, tetrahydrofuran and DMF. Heterocyclic rings (e.g., pyrrolidines, piperidines, homopiperidines, piperazines, homopiperazines, morpholines, and the like) are either commercially available or can be readily prepared by standard methods known within the art (see, for example, Louie and Hartwig, Tetrahedron Letters 36:3609, 1995; Guram et al., Angew Chem. Int. Ed. 34:1348, 1995). Alternatively, a compound of Formula (I) can be prepared by heating 3 with the heterocyclic ring in a suitable organic solvent such as THF, benzene, dioxane, toluene, alcohol, or mixtures thereof, under catalytic conditions using, for example, a palladium or copper catalyst (such as, but not limited to tris(dibenzylideneacetone) dipalladium(0) or copper (I) iodide) in the presence of a suitable base such as potassium carbonate, sodium t-butoxide, lithium hexamethyldisilizane, and the like.

Compounds of Formula (I) where R^{3a} is an indazole ring can be prepared by methods well known in the art. For example, copper catalyzed reaction of the appropriately substituted indazole with 3 (where X is halo) provides the appropriate compound of Formula (I). Alternatively, the bromoindazole undergoes palladium catalyzed reaction with compound 3 (X is halo) to provide a 4-(bromo-1H-indazol-1-yl) substituted compound of Formula (I). Subsequent N-arylation reaction with, for example morpholine or N-methylpiperazine provides the desired compound of Formula I. Alternatively, Suzuki-type reaction of the 4-(bromo-1H-indazol-1-yl)-substituted cinnoline compound with aryl or heteroaryl boronic acids (e.g., phenylboronic acid or 4-pyridine boronic acid) gives the corresponding 4-(aryl or heteroaryl substituted indazole)cinnoline compound of Formula (I).

Substituted indazoles useful to make compounds of Formula (I) are either commercially available (e.g., Aldrich Chemical Co., Sinova, Inc. (Bethesda, Md.), J & W PharmLab, LLC (Morrisville, Pa.)) or can be prepared by methods commonly known within the art (see, for example, Synthesis of 1-Aryl-1H-indazoles via Palladium-Catalyzed Intramolecular Amination of Aryl Halides, Lebedev, A. Y.; Khartulyari, A. S.; Voskoboynikov, A. Z. J. Org. Chem. 2005; 70(2); 596-602. and the references cited therein). For example, indazoles wherein R⁴ is heterocyclyl (e.g., morpholine or N-methylpiperazine) may be synthesized by Buchwald-type coupling of the corresponding bromoindazole with the desired heterocyclic compound. The bromoindazoles may be prepared as described in International Publication No. WO 2004/029050, the disclosure of which is incorporated herein by reference in its entirety.

Compounds of Formula (I) where X and Z are nitrogen, Y is carbon, and R¹, R², R³ and R^{3a} are as defined in the Summary of the Invention can be prepared as described in Scheme 4 below.

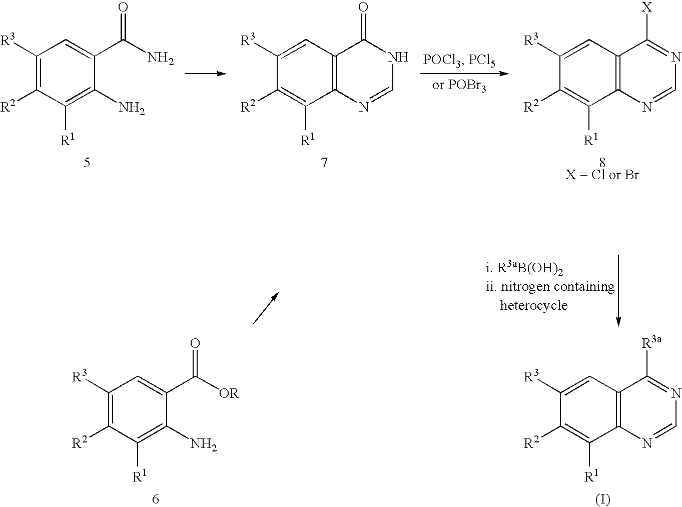

Reaction of 2-aminobenzamide compounds of formula 5 with trimethyl orthoformate or 2-aminobenzoic ester compounds of formula 6 with formamide in the presence of a base such as ammonium carbonate provides the corresponding 4-hydroxyquinazolone 7 which upon treatment with either phosphorous oxychloride or phosphorous oxybromide provides the corresponding chloro or bromo compound of formula 8. The chloro derivative 8 may be prepared by heating 7 in neat phosphorous oxychloride, followed by recrystallization of the product after neutralization (see, for example, Castle et al., J. Org. Chem. 17:1571, 1952). The bromo derivative 8 may be prepared by mixing a concentrated suspension of the 4-hydroxyquinazoline 7 in chloroform and phosphorous oxybromide at room temperature and then warming to reflux for 8 to 16 h. Extractive workup after neutralization and subsequent recrystallization from alcoholic solvent such as ethanol provides 4-bromoquinazoline 8. Compound 8 is then converted to a compound of Formula (I) as described in Scheme 1 above.

Compounds of formula 5 and 6 are either commercially available or can be synthesized by methods known in the art. Compounds of formula 5 where R¹ is hydrogen and R² and R³ are the same and are selected from cycloalkyloxy, cycloalkylalkyloxy, hydroxyalkyloxy, alkoxyalkyloxy, or —O-(alkylene)NR¹⁵R¹⁶, wherein R¹³, R¹⁴, R¹⁵, and R¹⁶ are independently hydrogen or alkyl, and wherein one or two carbon atoms in the alkyl chain in hydroxyalkyloxy, alkoxyalkyloxy, or —O-(alkylene)NR¹⁵R¹⁶ are optionally replaced by one to two oxygen or nitrogen atom(s), can be synthesized by methods known in the art. For example, 6,7-dimethoxy-4-quinazolone can be converted to 6,7-dihydroxy-4-quinazolone by treatment with BBr₃, which in turn can be treated with the desired R³LG where R³ is as defined above and LG is a suitable leaving group in the presence of a base such as cesium carbonate, triethylamine, sodium hydride, potassium carbonate, potassium hydride, and the like to provide the dialkylated product. Suitable organic solvents include acetone, acetonitrile, DMF, THF, and the like.

Compounds of formula 2 where R¹ is hydrogen and R² and R³ are independently cycloalkyloxy, cycloalkylalkyloxy, hydroxyalkyloxy, alkoxyalkyloxy, or —O-(alkylene)NR¹⁵R¹⁶, wherein R¹³, R¹⁴, R¹⁵, and R¹⁶ are independently hydrogen or alkyl, wherein one or two carbon atoms in the alkyl chain in hydroxyalkyloxy, alkoxyalkyloxy, or —O-(alkylene)NR¹⁵R¹⁶ are optionally replaced by one to two oxygen or nitrogen atom(s), and wherein R² and R³ are different, can be prepared from 6,7-dihydroxy-4-quinazolone as the benzyl ether (Greenspan et al., J. Med. Chem. 42:164, 1999) as described in Scheme 1 above.

Compounds of Formula (I) where Y and Z are nitrogen, X is CH, and R¹, R², R³ and R^{3a} are as defined in the Summary of the Invention can be prepared as described in Scheme 5 below. See, for example, Bioorg. Med. Chem. Lett., 2000, 10, 2235.

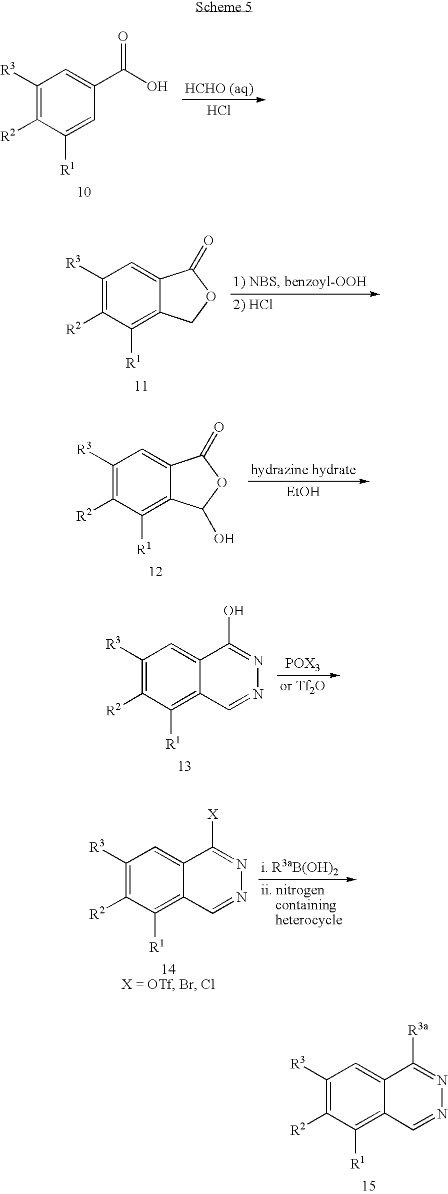

Treatment of a compound of formula 10 with aqueous formaldehyde and hydrochloric acid provides the cyclized ester 11. Compounds of formula 10 are either commercially available (e.g., 3,4-dimethoxybenzoic acid) or can be synthesized by methods common to the art (see, for example, Bioorg. Med. Chem. Lett., 2001, 11, 33). Oxidation of 11 with a suitable oxidizing agent such as perbenzoic acid in the presence of N-bromosuccinimide, followed by treatment with hydrazine, provides 4-hydroxyphthalazines 13. Treatment of 13 with phosphorous oxyhalide or with triflic anhydride as described in Scheme 1 above provides the 4-halo or triflyl phthalazines 14. Compound 14 is then converted to compound of Formula (I) where Y and Z are nitrogen and X is —CH═ as described in Scheme 1 above.

Compounds of Formula (I) where Y and Z are nitrogen, X is —CR═ where R is alkyl or halo, and R¹, R², R³ and R^{3a} are as defined in the Summary of the Invention can be prepared as described in Scheme 6 below. (see, for example, J. Med. Chem. 1996, 39, 343).

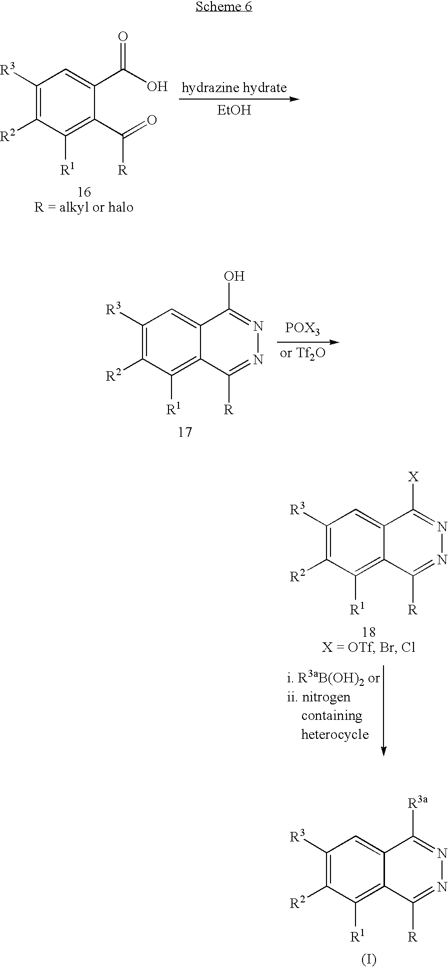

Treatment of 2-ketobenzoic acid (R is alkyl) or 2-carboxy acid halide (R is halo) of formula 16 with hydrazine hydrate provides 4-hydroxyphthalazine compound of formula 17. Compound 17 is then converted to a compound of Formula (I) as described in Scheme 1 above.

Compounds of Formula (I) where Y and Z are nitrogen, X is —CR═ where R is cyano, and R¹, R², R³ and R^{3a} are as defined in the Summary of the Invention can be prepared as described in Scheme 7.

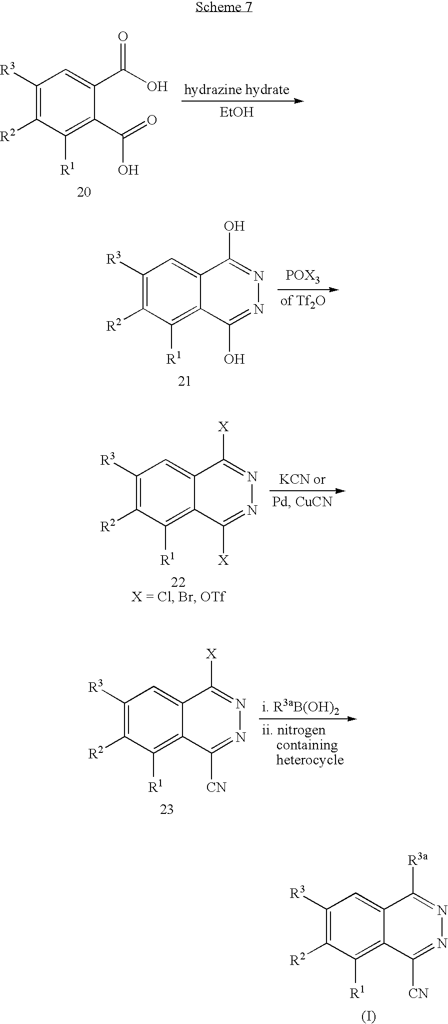

Treatment of a compound of formula 20 with hydrazine hydrate in an alcoholic solvent such as ethanol, and the like provides 2,4-dihydroxyphthalzine compound of formula 21. Halogenation of compound 21 with a suitable halogenating agent such as phosphorus oxychloride or bromide provides the di-halo compound of formula 22 where each X is halo, which, when R² and R³ are the same, may be converted to the nitrile substituted phthalazine intermediate 23 by reaction with one equivalent of potassium cyamide under nucleophilic reaction conditions, or by palladium catalyzed reaction in the presence of copper cyamide. Alternatively, 21 can be treated with triflic anhydride to provide a compound of formula 22 where each X is —OTf. The halo or triflate group at C-1 carbon is selectively replace by nitrile by reacting 22 with potassium cyamide or copper cyamide in presence of Pd catalyst to provide a compound of formula 23. Compound 23 is then converted to a compound of Formula (I) as described in Scheme 1 above.

Compounds of Formula (I) where Y and Z are nitrogen, X is —CR═ where R is cyano, and R¹, R², R³ and R^{3a} are as defined in the Summary of the Invention can be prepared as described in Scheme 8 below.

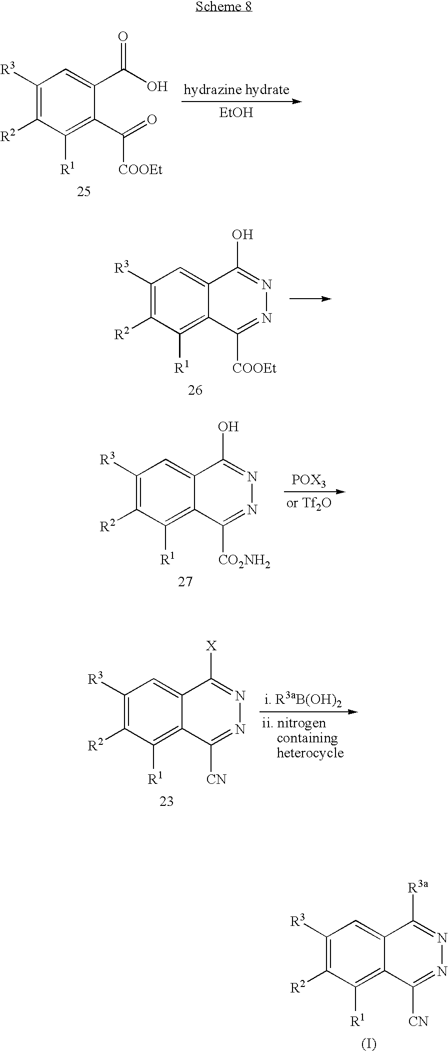

In an alternative method, compounds of formula 23 are prepared by cyclization of the oxalate compound 25 (readily produced by Friedel-Crafts acylation) with hydrazine to provide ester compound of formula 26. Compound 26 is converted to the corresponding amide compound of formula 27 by standard methods well known in the art. Simple dehydration of 27, concomitant with production of the halo phthalazine under treatment with phosphorous oxyhalide provides compound 23 which is then converted to a compound of Formula (I) as described in Scheme 1 above.

Utility and Methods of Use

In one aspect, methods are provided for treating a disorder or disease treatable by inhibition of PDE10 comprising administering a therapeutically effective amount of compound as provided herein to a patient in need thereof to treat the disorder or disease.

The compounds of the present invention inhibit PDE10 enzyme activity and hence raise the levels of cAMP or cGMP within cells that express PDE10. Accordingly, inhibition of PDE10 enzyme activity can be useful in the treatment of diseases caused by deficient amounts of cAMP or cGMP in cells. PDE10 inhibitors can also be of benefit in cases wherein raising the amount of cAMP or cGMP above normal levels results in a therapeutic effect. Inhibitors of PDE10 can be used to treat disorders of the peripheral and central nervous system, cardiovascular diseases, cancer, gastro-enterological diseases, endocrinological diseases and urological diseases.

Indications that may be treated with PDE10 inhibitors, either alone or in combination with other drugs, include, but are not limited to, those diseases thought to be mediated in part by the basal ganglia, prefrontal cortex and hippocampus. These indications include psychoses, Parkinson's disease, dementias, obsessive compulsive disorder, tardive dyskinesia, choreas, depression, mood disorders, impulsivity, drug addiction, attention deficit/hyperactivity disorder (ADHD), depression with parkinsonian states, personality changes with caudate or putamen disease, dementia and mania with caudate and pallidal diseases, and compulsions with pallidal disease.

Psychoses are disorders that affect an individual's perception of reality. Psychoses are characterized by delusions and hallucinations. The compounds of the present invention can be useful in treating patients suffering from all forms of psychoses, including, but not limited to, schizophrenia, late-onset schizophrenia, schizoaffective disorders, prodromal schizophrenia, and bipolar disorders. Treatment can be for the positive symptoms of schizophrenia as well as for the cognitive deficits and negative symptoms. Other indications for PDE10 inhibitors include psychoses resulting from drug abuse (including amphetamines and PCP), encephalitis, alcoholism, epilepsy, Lupus, sarcoidosis, brain tumors, multiple sclerosis, dementia with Lewy bodies, or hypoglycemia. Other psychiatric disorders, like posttraumatic stress disorder (PTSD), and schizoid personality can also be treated with PDE10 inhibitors.

Obsessive-compulsive disorder (OCD) has been linked to deficits in the frontal-striatal neuronal pathways. (Saxena S. et al., Br. J. Psychiatry Suppl., 1998; (35):26-37.) Neurons in these pathways project to striatal neurons that express PDE10. PDE10 inhibitors cause cAMP to be elevated in these neurons; elevations in cAMP result in an increase in CREB phosphorylation and thereby improve the functional state of these neurons. The compounds of the present invention therefore can be useful for the indication of OCD. OCD may result, in some cases, from streptococcal infections that cause autoimmune reactions in the basal ganglia (Giedd J N et al., Am J Psychiatry., 2000 February; 157(2):281-3). Because PDE10 inhibitors may serve a neuroprotective role, administration of PDE10 inhibitors may prevent the damage to the basal ganglia after repeated streptococcal infections and thereby prevent the development of OCD.

In the brain, the level of cAMP or cGMP within neurons is believed to be related to the quality of memory, especially long term memory. Without wishing to be bound to any particular mechanism, it is proposed that since PDE10 degrades cAMP or cGMP, the level of this enzyme affects memory in animals, for example, in humans. For example, a compound that inhibits cAMP phosphodiesterase (PDE) can thereby increase intracellular levels of cAMP, which in turn activate a protein kinase that phosphorylates a transcription factor (cAMP response binding protein), which transcription factor then binds to a DNA promoter sequence to activate genes that are important in long term memory. The more active such genes are, the better is long-term memory. Thus, by inhibiting a phosphodiesterase, long term memory can be enhanced.

Dementias are diseases that include memory loss and additional intellectual impairment separate from memory. The compounds of the present invention can be useful for treating patients suffering from memory impairment in all forms of dementia. Dementias are classified according to their cause and include: neurodegenerative dementias (e.g., Alzheimer's, Parkinson's disease, Huntington's disease, Pick's disease), vascular (e.g., infarcts, hemorrhage, cardiac disorders), mixed vascular and Alzheimer's, bacterial meningitis, Creutzfeld-Jacob Disease, multiple sclerosis, traumatic (e.g., subdural hematoma or traumatic brain injury), infectious (e.g., HIV), genetic (down syndrome), toxic (e.g., heavy metals, alcohol, some medications), metabolic (e.g., vitamin B12 or folate deficiency), CNS hypoxia, Cushing's disease, psychiatric (e.g., depression and schizophrenia), and hydrocephalus.

The condition of memory impairment is manifested by impairment of the ability to learn new information and/or the inability to recall previously learned information. The present invention includes methods for dealing with memory loss separate from dementia, including mild cognitive impairment (MCI) and age-related cognitive decline. The present invention includes methods of treatment for memory impairment as a result of disease. Memory impairment is a primary symptom of dementia and can also be a symptom associated with such diseases as Alzheimer's disease, schizophrenia, Parkinson's disease, Huntington's disease, Pick's disease, Creutzfeld-Jakob disease, HIV, cardiovascular disease, and head trauma as well as age-related cognitive decline. The compounds of the present invention would be useful in the treatment of memory impairment due to, for example, Alzheimer's disease, multiple sclerosis, amylolaterosclerosis (ALS), multiple systems atrophy (MSA), schizophrenia, Parkinson's disease, Huntington's disease, Pick's disease, Creutzfeld-Jakob disease, depression, aging, head trauma, stroke, spinal cord injury, CNS hypoxia, cerebral senility, diabetes associated cognitive impairment, memory deficits from early exposure of anesthetic agents, multiinfarct dementia and other neurological conditions including acute neuronal diseases, as well as HIV and cardiovascular diseases.

The compounds of the present invention are also suitable for use in the treatment of a class of disorders known as polyglutamine-repeat diseases. These diseases share a common pathogenic mutation. The expansion of a CAG repeat, which encodes the amino acid glutamine, within the genome leads to production of a mutant protein having an expanded polyglutamine region. For example, Huntington's disease has been linked to a mutation of the protein huntingtin. In individuals who do not have Huntington's disease, huntingtin has a polyglutamine region containing about 8 to 31 glutamine residues. For individuals who have Huntington's disease, huntingtin has a polyglutamine region with over 37 glutamine residues. Aside from Huntington's disease (HD), other known polyglutamine-repeat diseases and the associated proteins include dentatorubral-pallidoluysian atrophy, DRPLA (atrophin-1); spinocerebellar ataxia type-1 (ataxin-1); spinocerebellar ataxia type-2 (ataxin-2); spinocerebellar ataxia type-3 also called Machado-Joseph disease, MJD (ataxin-3); spinocerebellar ataxia type-6 (alpha 1a-voltage dependent calcium channel); spinocerebellar ataxia type-7 (ataxin-7); and spinal and bulbar muscular atrophy, SBMA, also know as Kennedy disease (androgen receptor).

The basal ganglia are important for regulating the function of motor neurons; disorders of the basal ganglia result in movement disorders. Most prominent among the movement disorders related to basal ganglia function is Parkinson's disease (Obeso J A et al., Neurology., 2004 Jan. 13; 62(1 Suppl 1):S17-30). Other movement disorders related to dysfunction of the basal ganglia include tardive dyskinesia, progressive supranuclear palsy and cerebral palsy, corticobasal degeneration, multiple system atrophy, Wilson disease, and dystonia, tics, and chorea. The compounds of the invention can be used to treat movement disorders related to dysfunction of basal ganglia neurons.

PDE10 inhibitors can be used to raise cAMP or cGMP levels and prevent neurons from undergoing apoptosis. PDE10 inhibitors may be anti-inflammatory by raising cAMP in glial cells. The combination of anti-apoptotic and anti-inflammatory properties, as well as positive effects on synaptic plasticity and neurogenesis, make these compounds useful to treat neurodegeneration resulting from any disease or injury, including stroke, spinal cord injury, Alzheimer's disease, multiple sclerosis, amylolaterosclerosis (ALS), and multiple systems atrophy (MSA).

Autoimmune diseases or infectious diseases that affect the basal ganglia may result in disorders of the basal ganglia including ADHD, OCD, tics, Tourette's disease, Sydenham chorea. In addition, any insult to the brain can potentially damage the basal ganglia including strokes, metabolic abnormalities, liver disease, multiple sclerosis, infections, tumors, drug overdoses or side effects, and head trauma. Accordingly, the compounds of the invention can be used to stop disease progression or restore damaged circuits in the brain by a combination of effects including increased synaptic plasticity, neurogenesis, anti-inflammatory, nerve cell regeneration and decreased apoptosis

The growth of some cancer cells is inhibited by cAMP and cGMP. Upon transformation, cells may become cancerous by expressing PDE10 and reducing the amount of cAMP or cGMP within cells. In these types of cancer cells, inhibition of PDE10 activity will inhibit cell growth by raising cAMP. In some cases, PDE10 may be expressed in the transformed, cancerous cell but not in the parent cell line. In transformed renal carcinoma cells, PDE1 is expressed and PDE10 inhibitors reduce the growth rate of the cells in culture. Similarly, breast cancer cells are inhibited by administration of PDE10 inhibitors. Many other types of cancer cells may also be sensitive to growth arrest by inhibition of PDE10. Therefore, compounds disclosed in this invention can be used to stop the growth of cancer cells that express PDE10.

The compounds of the invention are also suitable for use in the treatment of diabetes and related disorders such as obesity, by focusing on regulation of the cAMP signaling system. By inhibiting PDE-10A activity, intracellular levels of cAMP and increased, thereby increasing the release of insulin-containing secretory granules and, therefore, increasing insulin secretion. See, for example, WO 2005/012485, which is hereby incorporated by reference in its entirety. The compounds of Formula (I) can also be used to treat diseases disclosed in US Patent application publication No. 2006/019975, the disclosure of which is incorporated herein by reference in its entirety.

Testing

The PDE10 inhibitory activities of the compounds of the present invention can be tested using the in vitro and in vivo assays described in the Examples below.

Administration and Pharmaceutical Composition

In general, the compounds of this invention will be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors. Therapeutically effective amounts of compounds of formula (I) may range from approximately 0.1-1000 mg per day; preferably 0.5 to 250 mg/day, more preferably 3.5 mg to 70 mg per day.

In general, compounds of this invention will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen which can be adjusted according to the degree of affliction. Compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules are preferred) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of in general, a compound of formula (I) in combination with at least one pharmaceutically acceptable excipient. Acceptable excipients are non-toxic, aid administration, and do not adversely affect the therapeutic benefit of the compound of formula (I). Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc.

Other suitable pharmaceutical excipients and their formulations are described in Remington's Pharmaceutical Sciences, edited by E. W. Martin (Mack Publishing Company, 18th ed., 1990).

The level of the compound in a formulation can vary within the full range employed by those skilled in the art. Typically, the formulation will contain, on a weight percent (wt %) basis, from about 0.01-99.99 wt % of a compound of formula (I) based on the total formulation, with the balance being one or more suitable pharmaceutical excipients. Preferably, the compound is present at a level of about 1-80 wt %.

The compounds can be administered as the sole active agent or in combination with other pharmaceutical agents such as other agents used in the treatment of psychoses, especially schizophrenia and bipolar disorder, obsessive-compulsive disorder, Parkinson's disease, Alzheimer's disease, cognitive impairment and/or memory loss, e.g., nicotinic α-7 agonists, PDE4 inhibitors, other PDE10 inhibitors, calcium channel blockers, muscarinic m1 and m2 modulators, adenosine receptor modulators, ampakines, NMDA-R modulators, mGluR modulators, dopamine modulators, serotonin modulators, canabinoid modulators, and cholinesterase inhibitors (e.g., donepezil, rivastigimine, and galanthanamine). In such combinations, each active ingredient can be administered either in accordance with their usual dosage range or a dose below their usual dosage range and can be administered either simultaneously or sequentially.

Drugs suitable in combination with the compounds of the present invention include, but not limited to, other suitable schizophrenia drugs such as Clozaril, Zyprexa, Risperidone, and Seroquel, bipolar disorder drugs such as Lithium, Zyprexa, and Depakote, Parkinson's disease drugs such as Levodopa, Parlodel, Permax, Mirapex, Tasmar, Contan, Kemadin, Artane, and Cogentin, agents used in the treatment of Alzheimer's disease such as, but not limited to, Reminyl, Cognex, Aricept, Exelon, Akatinol, Neotropin, Eldepryl, Estrogen and Cliquinol, agents used in the treatment of dementia such as, but not limited to, Thioridazine, Haloperidol, Risperidone, Cognex, Aricept, and Exelon, agents used in the treatment of epilepsy such as, but not limited to, Dilantin, Luminol, Tegretol, Depakote, Depakene, Zarontin, Neurontin, Barbita, Solfeton, and Felbatol, agents used in the treatment of multiple sclerosis such as, but not limited to, Detrol, Ditropan XL, OxyContin, Betaseron, Avonex, Azothioprine, Methotrexate, and Copaxone, agents used in the treatment of Huntington's disease such as, but not limited to, Amitriptyline, Imipramine, Despiramine, Nortriptyline, Paroxetine, Fluoxetine, Setraline, Terabenazine, Haloperidol, Chloropromazine, Thioridazine, Sulpride, Quetiapine, Clozapine, and Risperidone; agents useful in the treatment of diabetes, including, but not limited to, PPAR ligands (e.g. agonists, antagonists, such as Rosiglitazone, Troglitazone and Pioglitazone), insulin secretagogues (for example, sulfonylurea drugs, such as Glyburide, Glimepiride, Chlorpropamide, Tolbutamide, and Glipizide, and non-sulfonyl secretagogues), α-glucosidase inhibitors (such as Acarbose, Miglitol, and Voglibose), insulin sensitizers (such as the PPAR-γ agonists, e.g., the glitazones; biguanides, PTP-1B inhibitors, DPP-IV inhibitors and 11beta-HSD inhibitors), hepatic glucose output lowering compounds (such as glucagon antagonists and metaformin, such as Glucophage and Glucophage XR), insulin and insulin derivatives (both long and short acting forms and formulations of insulin), and anti-obesity drugs, such as β-3 agonists, CB-1 agonists, neuropeptide Y5 inhibitors, Ciliary Neurotrophic Factor and derivatives (e.g., Axokine), appetite suppressants (e.g., Sibutramine), and lipase inhibitors (e.g., Orlistat).

### EXAMPLES

The following preparations and examples are given to enable those skilled in the art to more clearly understand and to practice the present invention. They should not be considered as limiting the scope of the invention, but merely as being illustrative and representative thereof.

### Synthetic Examples

### Example 1

### Synthesis of 4-bromo-7-methoxy-6-(2-methoxyethoxy)cinnoline

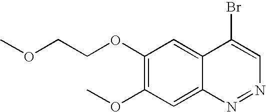

Step 1. Into a 1000 mL round bottom flask containing a solution of 1-(3-hydroxy-4-methoxy-phenyl)ethanone (11 g, 66.27 mmol) in acetone (200 mL) was added K₂CO₃ (39 g, 282.61 mmol), 1-bromo-2-methoxyethane (38 g, 273.40 mmol) and Cs₂CO₃ (3 g, 9.20 mmol). The resulting solution was stirred overnight while the temperature was maintained at reflux and the reaction progress was monitored by TLC (EtOAc/PE=1:1). The reaction mixture was concentrated (rotary evaporator), taken up in H2O and extracted with EtOAc. The combined organic layers were dried over Na₂SO₄ and concentrated (rotary evaporator) to provide 15.28 g of crude 1-(4-methoxy-3-(2-methoxyethoxy)phenyl)ethanone as a yellow solid.

Step 2. Into a 500 mL round bottom flask, containing a solution of 1-(4-methoxy-3-(2-methoxyethoxy)phenyl)ethanone (15.28 g, 68.21 mmol) in AcOH (200 mL) was added fuming HNO₃ (25 mL) dropwise over 30 minutes with stirring while cooling to a temperature of 0° C. Stirring continued overnight at room temperature and the reaction progress was monitored by TLC (EtOAc/PE=1:2). Upon completion, 500 mL of H₂O/ice was added and the resulting solution was extracted with EtOAc. The organic layers were combined, washed with aqueous NaHCO₃, dried over Na₂SO₄ and concentrated (rotary evaporator). The residue was purified by silica gel chromatography using 1:10 EtOAc/PE as eluant to provide 7.3 g of 1-(4-methoxy-5-(2-methoxyethoxy)-2-nitrophenyl)ethanone as a green-yellow solid.

Step 3. A 500 mL round bottom flask containing a solution of 1-(4-methoxy-5-(2-methoxyethoxy)-2-nitrophenyl)ethanone (7.3 g, 27.14 mmol) in CH₃OH (200 mL) and Pd/C (2.9 g) was purged, flushed and maintained with a hydrogen atmosphere. The resulting mixture was stirred for 4.5 hours and the reaction progress was monitored by TLC (EtOAc/PE=1:2). Upon completion the mixture was filtered and concentrated (rotary evaporator) to provide 6.2 g of 1-(2-amino-4-methoxy-5-(2-methoxyethoxy)phenyl)ethanone as a brown solid.

Step 4. 1-(2-Amino-4-methoxy-5-(2-methoxyethoxy)phenyl)ethanone (6.2 g, 25.94 mmol), H₂O (22 mL) and concentrated HCl (165 mL) were combined in a 500 mL round bottom flask and cooled to 0° C. A solution of NaNO₂ (1.97 g, 28.55 mmol) in H₂O (8.7 ml) was added drop-wise over 30 minutes with stirring at −5 to 0° C. The mixture was stirred for 1.5 hours at room temperature and then for 4.5 hours at 70° C. and the reaction progress was monitored by TLC(CH₂Cl₂/MeOH=10:1). The reaction mixture was cooled in a refrigerator and the product isolated by filtration. The residue was dissolved in 40 mL of 15% NaOH solution, filtered, and the pH was adjusted to 7 by the addition of 37% HCl. The product was isolated by filtration and the filter cake dried in an oven under reduced pressure to provide 4.7 g of 7-methoxy-6-(2-methoxyethoxy)cinnolin-4-ol as a grey white solid.

Step 5. Into a 500 mL round bottom flask purged and maintained with an inert atmosphere of nitrogen, was added 7-methoxy-6-(2-methoxyethoxy)cinnolin-4-ol (3.22 g, 12.88 mmol), CH₃CN (240 mL) and POBr₃ (7.39 g, 25.75 mmol). The resulting mixture was stirred for 5 hours at 70° C. and the reaction progress was monitored by TLC(CH₂Cl₂/MeOH=15:1). The reaction mixture was quenched by the addition of 400 mL of H₂O/ice and then the pH was adjusted to 7 by the addition of NaHCO₃ (8%), and the mixture was extracted with CH₂Cl₂. The organic layers were combined, washed with 40 mL of brine, dried over Na₂SO₄ and concentrated (rotary evaporator) to provide 2.95 g (52%) of 4-bromo-7-methoxy-6-(2-methoxyethoxy)cinnoline as a brown solid. ¹HNMR (300 MHz, CDCl₃) δ3.51 (3H, s), 3.91-3.94 (2H, m), 4.10 (3H, s), 4.39-4.40 (2H, m), 7.28 (2H, s), 9.27 (1H, s). LCMS [M+H]⁺ calcd for C₁₂H₁₄BrN₂O₃ 313, found 313.

### Example 2

### Synthesis of 2-(4-methoxyphenyl)-3-methylmorpholine

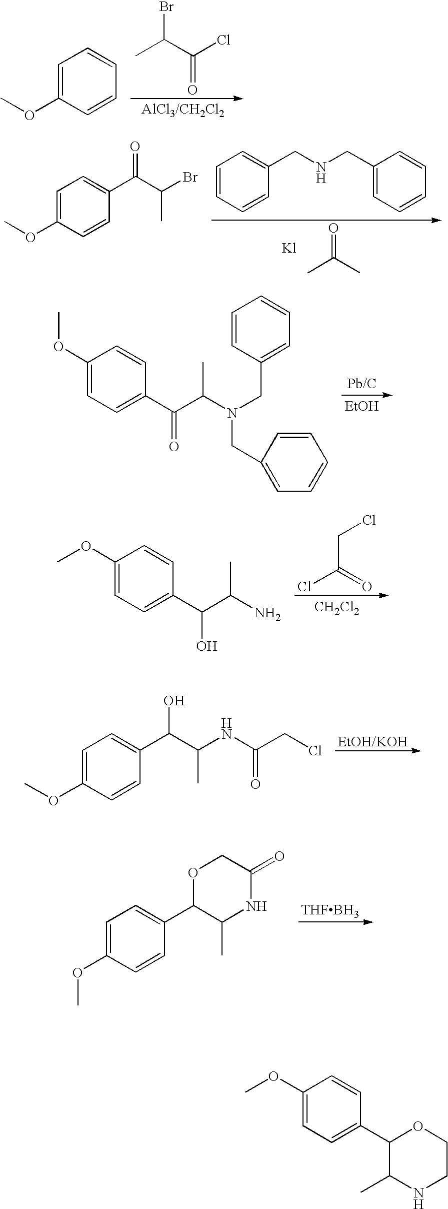

Step 1. Into a 1000 mL 4-necked round bottom flask purged and maintained with an inert atmosphere of nitrogen containing a solution of AlCl₃ (160.2 g, 1.20 mol) in CH₂Cl₂ (50 mL) was added a solution of anisole (64.8 g, 599.44 mmol) in CH₂Cl₂ (50 mL) dropwise with stirring at 0° C. over a 30 minute period. This was followed by the drop-wise addition of a solution of 2-bromopropanoyl chloride (128.5 g, 749.71 mmol) in CH₂Cl₂ (200 mL) with stirring at 0° C. over 60 minutes. The resulting solution was stirred for 0.5 hours at 0° C. and then for 2 hours at room temperature. The reaction mixture was quenched by the addition of 1000 mL of HCl/H₂O/ice and then extracted three times with CH₂Cl₂, the organic fractions were combined, dried over MgSO₄ and concentrated. The residue was purified by silica gel chromatography using 1:100 EtOAc/PE as eluant to provide 25 g of crude 2-bromo-1-(4-methoxyphenyl)propan-1-one as yellow oil.

Step 2. 2-Bromo-1-(4-methoxyphenyl)propan-1-one (12 g, 49.36 mmol), dibenzylamine (19.4 g, 98.33 mmol), acetone (600 mL) and KI (370 mg, 2.23 mmol) were combined in a 1000 mL round bottom flask and stirred for 3 days at room temperature. The reaction mixture was filtered, the filtrate was concentrated and the residue was purified by silica gel chromatography using 1:100 EtOAc/PE as eluant to provide 12.8 g of 2-(dibenzylamino)-1-(4-methoxyphenyl)propan-1-one as a white solid.

Step 3. Into a 100 mL round bottom flask purged, flushed and maintained with a hydrogen atmosphere was added 2-(dibenzylamino)-1-(4-methoxyphenyl)propan-1-one (3 g, 8.34 mmol), Pd/C (3 g), EtOH (75 mL) and HCl (0.6 mL). The reaction mixture was stirred overnight at room temperature, filtered and the filtrate was concentrated to provide 1.4 g of 2-amino-1-(4-methoxyphenyl)propan-1-ol as a white solid.

Step 4. Into a mixture of 2-amino-1-(4-methoxyphenyl)propan-1-ol (3.1 g, 17.11 mmol), NaOH (1.0 g), 5 drops of water and CH₂Cl₂ (mL) was added a solution of 2-chloroacetyl chloride (2.9 g, 25.7 mmol) in CH₂Cl₂ (15 mL) drop-wise with stirring at 0° C. over a 15 minute period. The reaction mixture was stirred for 1.5 hours at 0° C. in a bath of H₂O/ice and then washed with HCl/H₂O, NaHCO₃/H₂O, dried over MgSO₄ and concentrated to provide 3.3 g of 2-chloro-N-(1-hydroxy-1-(4-methoxyphenyl)propan-2-yl)acetamide as a white solid.

Step 5. 2-Chloro-N-(1-hydroxy-1-(4-methoxyphenyl)propan-2-yl)acetamide (670 mg, 2.60 mmol), KOH (0.56 g) and EtOH (70 mL) were combined and stirred for 2.5 hours at room temperature. The reaction mixture was concentrated, diluted with 10 mL of H₂O and extracted with CH₂Cl₂. The organic layers were combined, dried over MgSO₄ and concentrated to provide 0.33 g of 6-(4-methoxyphenyl)-5-methylmorpholin-3-one as a white solid.

Step 6. A solution of 6-(4-methoxyphenyl)-5-methylmorpholin-3-one (330 mg, 1.49 mmol) in THF (50 mL) contained in a 100 mL 3-necked round bottom flask purged and maintained with an inert atmosphere of nitrogen was treated with THF.BH₃ (15 mL) in several batches while cooling to 0° C. over a period of 10 minutes. Stirring was continued for 3 hours at room temperature and the reaction progress was monitored by TLC (CH₂Cl₂/MeOH=10:1). The reaction mixture was quenched by adding 10 mL of MeOH. The reaction mixture was concentrated, diluted with 30 mL of 110% HCl/H₂O and warmed to 80° C. for 0.5 hours. The pH was adjusted to 10 by the addition of NaOH (20% aq. solution), extracted with EtOAc, dried over Na₂SO₄ and concentrated to provide 0.3 g of 2-(4-methoxyphenyl)-3-methylmorpholine as a light yellow liquid. LCMS [M+H]⁺ calcd for C₁₂H₁₈NO₂ 208, found 208.

### Example 3

### Synthesis of 4-bromo-6-ethyl-7-methoxycinnoline

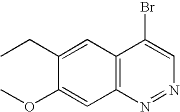

Step 1. Into a 250 mL 3-necked round bottom flask, was placed fuming HNO₃ (20 mL). To this is added concentrated sulfuric acid (28 mL). 1-Ethylbenzene (15 g, 141.51 mmol) was added dropwise with stirring, maintaining the temperature below 95° C. The resulting solution was poured into iced water and the product was extracted using ethyl acetate. The combined organics were dried (MgSO₄) and concentrated. The residue was purified by eluting through a column with a 1:10 ethyl acetate/petroleum ether solvent system to afford 22 g of 1-ethyl-2,4-dinitrobenzene as a yellow oil.

Step 2. Iron (25.8 g, 460.71 mmol) was added in several portions to a solution of 1-ethyl-2,4-dinitrobenzene (30 g, 137.76 mmol, prepared as described in Step 1 above) in acetic acid (350 mL), while maintaining the temperature at reflux. The resulting solution was maintained at reflux for a further 10 min. The product was precipitated by the addition of ice, and the product was extracted with ethyl acetate. The organic layers were combined, dried (MgSO₄), filtered, and concentrated. The residue was purified by eluting through a column with a 1:10 ethyl acetate/petroleum ether solvent system to afford 12.9 g of 2-ethyl-5-nitrobenzenamine as a brown solid.

Step 3. A solution of sulfuric acid (98%, 39 g, 390.00 mmol) in water (160 mL) was added to 2-ethyl-5-nitrobenzenamine (12.9 g, 69.94 mmol, prepared as described in Step 2 above). The mixture was cooled to 0-5° C., and a solution of sodium nitrite (5.63 g, 81.59 mmol) in water (20 mL) was then added. The resulting solution was maintained for 30 minutes at 0-5° C. Sulfuric acid (65%, 600 g, 3.98 mol) was then added, and the temperature was maintained at reflux for 1 hr. The reaction mixture was cooled in a bath of iced water, and the product was extracted with ethyl acetate. The organic layers were combined and washed with aqueous saturated sodium bicarbonate and brine. The solution was dried (MgSO₄), filtered and concentrated. The residue was purified by eluting through a column with a 1:10 ethyl acetate/petroleum ether solvent system to afford 7.65 g of 2-ethyl-5-nitrophenol as a red solid.

Step 4. Potassium carbonate (12.6 g, 91.30 mmol) was added to a solution of 2-ethyl-5-nitrophenol (7.65 g, 36.65 mmol, prepared as described in Step 3 above) in acetone (200 mL). Methyl iodide (19.5 g, 137.32 mmol) was then added, and the resulting solution was maintained at reflux for 3 hr. The solution was allowed to cool, filtered and concentrated. The residue was purified by eluting through a column with a 1:20 ethyl acetate/petroleum ether solvent system to afford 5.15 g of 1-ethyl-2-methoxy-4-nitrobenzene as yellow oil.

Step 5. A mixture of ammonium chloride (15.2 g, 284.11 mmol) in water (100 mL) was added to a solution of 1-ethyl-2-methoxy-4-nitrobenzene (5.15 g, 25.61 mmol, prepared as described above in step 4) in ethanol (100 mL). The mixture was cooled to 0-5° C. and zinc (7.40 g, 113.85 mmol) was added in several portions. Acetic acid (6.83 g, 113.83 mmol) was then added dropwise at 0-5° C. The resulting solution was stirred at room temperature for 3 hr. The mixture was concentrated and sodium bicarbonate was added to adjust the pH to 7. The resulting solution was extracted with ethyl acetate and the organic layers were combined, washed with brine, dried (MgSO₄), filtered and concentrated. The residue was purified by eluting through a column with a 1:5 ethyl acetate/petroleum ether solvent system to afford 3.1 g of 4-ethyl-3-methoxybenzenamine as a green solid.

Step 6. Triethylamine (2.28 g, 22.57 mmol) was added to a solution of 4-ethyl-3-methoxybenzenamine (3.1 g, 19.50 mmol, prepared as described in Step 5 above) in methylene chloride (100 mL). Acetyl chloride (2.42 g, 30.83 mmol) was then added dropwise at 0-5° C., and the mixture was maintained at this temperature for 30 minutes. The mixture was concentrated and the product was extracted with ethyl acetate. The organics layers were combined, dried (MgSO₄), filtered and concentrated. The residue was purified by eluting through a column with a 1:2 ethyl acetate/petroleum ether solvent system to afford 2.8 g of N-(4-ethyl-3-methoxyphenyl)acetamide as a pink solid.

Step 7. Aluminum (III) chloride (7.7 g, 58.11 mmol) was added to a solution of N-(4-ethyl-3-methoxyphenyl)acetamide (2.8 g, 13.06 mmol, prepared as described in Step 6 above) in dichloromethane (100 mL). Acetyl chloride (2.3 g, 29.30 mmol) was then added dropwise at 0-5° C. and the resulting solution was maintained at room temperature for 2 hr. Ice (100 g) was added, and the resulting solution was extracted with methylene chloride. The organic layers were combined, washed with saturated sodium bicarbonate and brine, dried (MgSO₄), filtered and concentrated to afford 3.6 g of N-(2-acetyl-4-ethyl-5-methoxyphenyl)acetamide as a red solid.

Step 8. Hydrochloric acid (100 mL) was added to a solution of N-(2-acetyl-4-ethyl-5-methoxyphenyl)acetamide (3.6 g, 12.26 mmol, prepared as described in Step 7 above) in 1,4-dioxane (100 mL). The resulting solution was maintained at 85° C. for 3 hr. The mixture was concentrated and sodium bicarbonate was added to adjust the pH of the solution to 7. The product was extracted with ethyl acetate. The organic layers were combined, washed with brine, dried (MgSO₄) and concentrated. The residue was purified by eluting through a column with a 1:20 ethyl acetate/petroleum ether solvent system to afford 1.8 g of 1-(2-amino-5-ethyl-4-methoxy phenyl)ethanone as a light yellow solid.

Step 9. A solution of sodium nitrite (380 mg, 5.51 mmol) in water (5 mL) was added dropwise to a chilled (0-5° C.) solution of 1-(2-amino-5-ethyl-4-methoxyphenyl)ethanone (1 g, 4.66 mmol, prepared as described above in Step 8) in 12 M hydrochloric acid (50 mL). The resulting solution was maintained at room temperature for 16 hr. The pH of the mixture was adjusted to 7 by the addition of sodium bicarbonate. The product was extracted with ethyl acetate and the combined organics were washed with brine, dried (MgSO₄), filtered and concentrated to afford 400 mg of 6-ethyl-7-methoxycinnolin-4-ol as a pink solid.

Step 10. Phosphoryl tribromide (2.1 g, 7.32 mmol) was added to a solution of 6-ethyl-7-methoxycinnolin-4-ol (480 mg, 2.12 mmol, prepared as described in Step 9 above) in acetonitrile (100 mL) and the resulting solution was maintained at 70° C. for 3 hr. The pH of the mixture was adjusted to 7 by the addition of sodium bicarbonate. The mixture was concentrated and the product was extracted with ethyl acetate. The organic layers were combined, washed with brine, dried (MgSO₄), filtered and concentrated. The residue was purified by eluting through a column with a 1:2 ethyl acetate/petroleum ether solvent system to afford 200 mg of 4-bromo-6-ethyl-7-methoxycinnoline as a pink solid.

To synthesize starting materials where R² is, for example, methoxyethoxy (for instance, as in 4-bromo-6-ethyl-7-methoxyethoxycinnoline) or cyclopropoxy (for instance, as in 4 bromo-6-ethyl-7-cyclopropoxycinnoline), step 4 in this Example could be modified by replacing the methyl iodide with an equal molar amount of, for example, 1-bromo-2-methoxyethane or bromocyclopropane respectively.

### Example 4

### Synthesis of 4-bromo-7-ethyl-6-methoxycinnoline

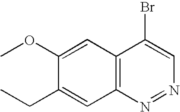

Step 1. Aluminum (III) chloride (27 g, 202.49 mmol) was added to a chilled (−70° C.) solution of 1-ethylbenzene (10.6 g, 99.85 mmol) in methylene chloride (100 mL). A solution of acetic anhydride (10.2 g, 99.91 mmol) in methylene chloride (20 mL) was added dropwise over 3 hours, while maintaining the temperature at −70° C. The resulting solution was maintained for 2 hours between −70 and −50° C., then added to a mixture of ice (200 mL) and hydrochloric acid (100 mL). The product was extracted with methylene chloride and the organic layers were combined, washed with 10% aqueous sodium bicarbonate solution and brine, dried, filtered and concentrated to afford 15 g of 1-(4-ethylphenyl)ethanone as a colorless liquid.

Step 2. 1-(4-Ethylphenyl)ethanone (15 g, 86.03 mmol, prepared as described in Step 1 above) was added dropwise to chilled (0-5° C.) concentrated sulfuric acid (20 mL). A solution of fuming nitric acid (8.1 g) in concentrated sulfuric acid (10 mL) was then added dropwise and the mixture was maintained for 15 minutes at 0-5° C., then added slowly to 300 mL iced water. The product was extracted with methylene chloride. The organic layers were combined, washed with saturated sodium bicarbonate and brine (200 mL), dried, filtered and concentrated. The residue was purified by eluting through a column with a 1:50 ethyl acetate/petroleum ether solvent system to afford 14 g of 1-(4-ethyl-3-nitrophenyl)ethanone as a yellow liquid.

Step 3. A solution of 1-(4-ethyl-3-nitrophenyl)ethanone (10 g, 49.17 mmol, prepared as described in Step 2 above) in acetic acid (10 mL) was added in several portions to a mixture of iron (8.2 g, 146.82 mmol) in water (100 mL), while warming the mixture to a temperature of 80-90° C. The resulting solution was maintained at reflux for 1.5 hr. The mixture was adjusted to pH 7-8 by the addition of ammonia (28%) and was filtered. The product was extracted with methylene chloride (3×100 mL) and the organic layers were combined, washed with brine, dried (Na₂SO₄), filtered and concentrated to afford 8.6 g of 1-(3-amino-4-ethylphenyl)ethanone as a yellow liquid.

Step 4. 1-(3-amino-4-ethylphenyl)ethanone (8.6 g, 44.79 mmol, prepared as described in Step 3 above) was added to chilled (0° C.) 20% sulfuric acid (80 mL). Sodium nitrite (4.5 g, 65.22 mmol) in water (20 mL) was then dropwise maintaining a temperature of 0-5° C. The resulting solution was allowed to react for 1 hour at 0-5° C. Urea (1.6 g, 26.64 mmol) was then added and the resulting solution was maintained for 15 minutes 0-5° C. This solution was then added dropwise to 30% sulfuric acid (100 mL) while heating to a temperature of 100° C. The resulting solution was maintained at 100° C. for a further 15 minutes and then cooled and filtered. The filter cake was washed with 10% sodium bicarbonate. The solid was dried to afford 6.8 g of 1-(4-ethyl-3-hydroxyphenyl)ethanone as a yellow solid.

Step 5. Propan-2-one (50 mL) and potassium carbonate (8.3 g, 60.14 mmol) were added to 1-(4-ethyl-3-hydroxyphenyl)ethanone (6.6 g, 38.23 mmol, prepared as described in Step 4 above). Methyl iodide (17.1 g, 120.42 mmol) was then added and the resulting solution was maintained at 60° C. for 3 hr. The mixture was concentrated and diluted with water (100 ml). The product was extracted with methylene chloride. The organic layers were combined and dried over Na₂SO₄. The residue was purified by eluting through a column with a 1:20 ethyl acetate/petroleum ether solvent system to afford 7 g of 1-(4-ethyl-3-methoxyphenyl)ethanone as a yellow liquid.

Step 6. Acetic acid (1 mL) was added to 1-(4-ethyl-3-methoxyphenyl)ethanone (300 mg, 1.69 mmol, prepared as described in Step 5 above). The mixture was chilled to 0-5° C. and fuming nitric acid (1 mL) was added. The resulting was maintained at room temperature for 2 hr, and then cooled in iced water. The product was extracted with methylene chloride. The organic layers were combined, washed with 10% sodium bicarbonate solution and brine, dried (Na₂SO₄), filtered and concentrated. The residue was purified by eluting through a column with a 1:50 ethyl acetate/petroleum ether solvent system to afford 100 mg of 1-(4-ethyl-5-methoxy-2-nitrophenyl)ethanone as a yellow solid.

Step 7. A solution of 1-(4-ethyl-5-methoxy-2-nitrophenyl)ethanone (250 mg, 1.12 mmol, prepared as described above in Step 6) in acetic acid (2 mL) was added to a mixture of iron (200 mg, 3.58 mmol) in water (30 ml). The resulting mixture was heated to reflux temperature for 45 minutes. The pH was adjusted to 8 by the addition of ammonia (28%) and the mixture was filtered. The product was extracted with ethyl acetate and the organic layers were combined, dried (Na₂SO₄) and concentrated to afford 200 mg of 1-(2-amino-4-ethyl-5-methoxyphenyl)ethanone as a yellow liquid.

Step 8. Sodium nitrite (250 mg, 3.62 mmol) in water (5 ml) was added to a chilled (0-5° C.) solution of 1-(2-amino-4-ethyl-5-methoxyphenyl)ethanone (500 mg, 2.46 mmol, prepared as described above in Step 7) in concentrated hydrochloric acid (10 mL). The resulting solution was maintained at 0-5° C. for 15 minutes. Iced water (50 mL) was then added, and the pH was adjusted to 6-7 by the addition of sodium carbonate solution (10%). The product was extracted with ethyl acetate and the organic layers were combined, dried (Na₂SO₄) and concentrated. The residue was purified by eluting through a column with a 1:1 ethyl acetate/petroleum ether solvent system to afford 300 mg of 7-ethyl-6-methoxycinnolin-4-ol as a brown solid.

Step 9. Phosphoryl tribromide (1.4 g, 4.88 mmol) was added to a solution of 7-ethyl-6-methoxycinnolin-4-ol (300 mg, 1.47 mmol, prepared as described above in Step 8) in acetonitrile (20 mL) and the resulting solution was maintained at 70° C. for 3 hr. Iced water (30 mL) was then added. The pH was adjusted to 6-7 by the addition of sodium carbonate (10% solution) and the product was extracted with ethyl acetate. The organic layers were combined, dried (Na₂SO₄), filtered and concentrated. The residue was purified by eluting through a column with a 1:8 ethyl acetate/petroleum ether solvent system to afford 150 mg of 4-bromo-7-ethyl-6-methoxycinnoline as a light yellow solid. ¹H NMR (400 MHz, CDCl₃) δ 1.36 (t, 3H), 2.88 (q, 2H), 4.08 (s, 3H), 7.21 (s, 1H), 8.29 (s, 1H), 9.31 (s, 1H).

Demethylation of the methoxy group with a suitable agent such as BBr₃/HCl would provide the corresponding 7-hydroxyl analog which can then be reacted with 1-bromo-2-methoxyethane to provide 4-bromo-7-ethyl-6-(2-methoxyethoxy)cinnoline.

To synthesize starting material where R³ is, for example, methoxyethoxy or cyclopropoxy; step 5 in this Example could be modified by replacing the methyl iodide with an equal molar amount of, for example, 1-bromo-2-methoxyethane or bromocyclopropane respectively.

### Example 5

### Synthesis of 1-(6,7-dimethoxycinnolin-4-yl)piperidin-4-amine

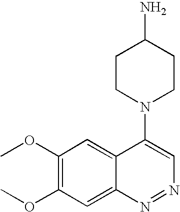

A mixture of 4-bromo-6,7-dimethoxycinnoline (0.5 g, 0.002 mol), 4-BOC-amino-piperidine (0.5619 g, 2.806 mmol), tris(dibenzylideneacetone)dipalladium(0) (0.0891 g, 0.0973 mmol), 9,9-dimethyl-4,5-bis(diphenylphosphino)xanthene (0.110 g, 0.190 mmol), sodium tert-butoxide (0.268 g, 2.79 mmol) and toluene (4.0 mL, 0.037 mol) was heated at 50° C. overnight. The reaction mixture was flushed through an SCX column, washed with methanol and eluted with 2.0 M ammonia/methanol. The product was purified by silica gel chromatography on a 40 g column using a gradient going from 100% CH₂Cl₂ to 50% (8:1:1 CH₂Cl₂/MeOH/7M NH₃ in MeOH)/CH₂Cl₂ as elutant to provide 1-(6,7-dimethoxycinnolin-4-yl)piperidin-4-amine.

Proceeding as described in Example 5 above, but substituting 4-bromo-6,7-dimethoxy-cinnoline with 4-bromo-7-methoxy-6-(2-methoxyethoxy)cinnoline would provide 1-[7-methoxy-6-(2-methoxyethoxy)cinnolin-4-yl]piperidin-4-amine.

### Example 6

### Synthesis of 7-methoxy-6-(2-methoxyethoxy)-4-[2-(4-methoxyphenyl)morpholin-4-yl]cinnoline

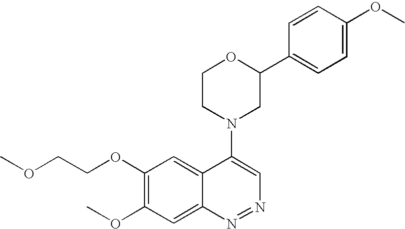

4-Bromo-7-methoxy-6-(2-methoxyethoxy)cinnoline (100 mg, 0.32 mmol), 2-(4-methoxyphenyl)morpholine (74 mg, 0.38 mmol), tetrahydrofuran (5.5 mL), 2-dicyclohexyl-phosphino-2′,4′,6′-tri-1-propyl-1,1′-biphenyl (17.3 mg, 0.036 mmol), sodium tert-butoxide (92 mg, 0.958 mmol) and tris(dibenzylideneacetone)dipalladium(0) (17.3 mg, 0.019 mmol) were combined and stirred for 15 hours at 85° C. The mixture was filtered through celite, taken up in 100 mL of DCM and washed with 1×40 mL of saturated aqueous sodium bicarbonate. The organic fraction was concentrated and purified by column chromatography using a gradient elution going from 3% to 8% MeOH in 1:1 EtOAc/hexane and 0.3% DEMA to give 7-methoxy-6-(2-methoxyethoxy)-4-[2-(4-methoxyphenyl)morpholin-4-yl]cinnoline as a yellow gum. LC/MS: M+H 426.1.

Exemplary compounds described in Examples 7-40 can be prepared, for instance, using 4-bromo-7-methoxy-6-(2-methoxyethoxy)cinnoline prepared as described in Example 1.

### Example 7

### Synthesis of 4-(1,3-benzoxazol-2-yl)-7-methoxy-6-(2-methoxyethoxy)cinnoline

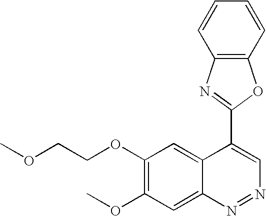

n-Butyllithium (0.0639 g, 0.997 mmol) is added dropwise over 30 minutes to a chilled (−30° C.) solution of benzoxazole (0.119 g, 0.997 mmol) in N,N-dimethylacetamide (3 mL). Tris(dibenzylideneacetone)dipalladium(0) (0.046 g, 0.050 mmol) and a solution of 4-bromo-7-methoxy-6-(2-methoxyethoxy)cinnoline (0.498 mmol) in N,N-dimethylacetamide (3 mL) is added. The resulting mixture is heated to 85° C. for 8 h, then cooled to room temperature. The solvent is evaporated and the residue is diluted with ethyl acetate. The solution is filtered through celite, washed with aqueous sodium bicarbonate, and then concentrated. The crude product is purified by column chromatography (gradient elution using 0-5% methanol/dichloromethane).

### Example 8

### Synthesis of N-cyclopropyl-1-[7-methoxy-6-(2-methoxyethoxy)cinnolin-4-yl]-1H-indazole-3-carboxamide

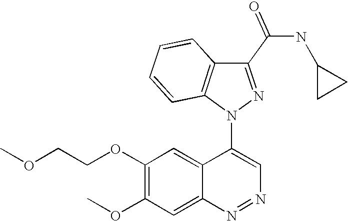

Step 1. n-Butyllithium (0.13 g, 0.0020 mol) is added dropwise over 30 minutes to a chilled (−30° C.) solution of 1H-indazole-3-carboxylic acid (0.162 g, 0.999 mmol) in N,N-dimethylacetamide (3 mL). A solution of tris(dibenzylideneacetone)dipalladium(0) (0.083 g, 0.091 mmol), 4-bromo-7-methoxy-6-(2-methoxyethoxy)cinnoline (0.908 mmol) and triethylamine (380 μL) in N,N-dimethylacetamide (3 mL) is added and the reaction mixture is raised to 25° C. for 5 minutes, then to 85° C. for 2 hours. The solvent is removed by evaporation and the residue is diluted with 20% methanol/dichloromethane (50 mL), filtered through celite and concentrated. Purification by column chromatography (gradient elution using 30-60% methanol/ethyl acetate) gives 1-(7-methoxy-6-(2-methoxyethoxy)cinnolin-4-yl)-1H-indazole-3-carboxylic acid.

Step 2. A mixture of 1-(7-methoxy-6-(2-methoxyethoxy)cinnolin-4-yl)-1H-indazole-3-carboxylic acid (0.08 mmol; Step 1 above), cyclopropylamine (0.171 mol), N,N′-diisopropylcarbodiimide (21.4 μL), 1-hydroxybenzotriazole (5.8 mg, 0.043 mol), and N,N-dimethylformamide (2.00 mL) is stirred at room temperature for 8 h. The solvent is then evaporated. The resulting residue is dissolved in ethyl acetate, and the solution is washed with aqueous sodium bicarbonate and concentrated.

### Example 9

### Synthesis of 7-methoxy-6-(2-methoxyethoxy)-4-[4-(2-methoxyethoxy)-1H-indazol-1-yl]cinnoline

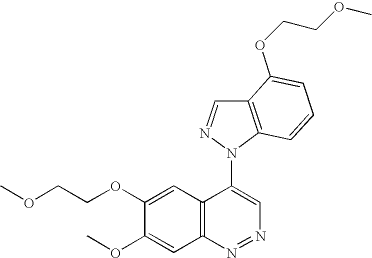

Into a 5 mL microwave tube is added 4-bromo-7-methoxy-6-(2-methoxyethoxy)cinnoline (0.743 mmol), 4-(2-methoxyethoxy)-1H-indazole (171.0 mg, 0.8895 mmol), copper (I) iodide (28 mg, 0.15 mmol), potassium carbonate (206.7 mg, 1.496 mmol), N,N′-dimethyl-1,2-ethanediamine (32 μL) and toluene (6.00 mL). The suspension is heated at 115° C. for 24 h. The crude product is purified by preparative HPLC (using a gradient elution 10:90 to 80:20 acetonitrile:water with 0.1% formic acid and a flow rate of 45 mL/min).

### Example 10

### Synthesis of 7-methoxy-6-(2-methoxyethoxy)-4-(6-morpholin-4-yl-1H-indazol-1-yl)cinnoline

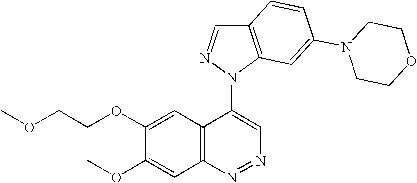

Step 1. Into a 5 mL microwave tube is added 4-bromo-7-methoxy-6-(2-methoxyethoxy)cinnoline (0.743 mmol), 6-bromo-1H-indazole (219.1 mg, 1.112 mmol), copper(I) iodide (18 mg, 0.093 mmol), potassium carbonate (258.4 mg, 1.870 mmol), N,N′-dimethyl-1,2-ethanediamine (40 μL) and toluene (1 mL) The resulting suspension is heated at 115° C. for 24 h. The crude product is purified by flash chromatography on silica gel (using a gradient of 50% ethyl acetate/hexanes to 100% hexanes) to give 4-(6-bromo-1H-indazol-1-yl)-7-methoxy-6-(2-methoxyethoxy)cinnoline.

Step 2. Into a 10 ml sealed microwave tube is added 4-(6-bromo-1H-indazol-1-yl)-7-methoxy-6-(2-methoxyethoxy)cinnoline (0.260 mmol), morpholine (34.0 μL, 0.389 mmol), tetrahydrofuran (5.0 mL), tris(dibenzylideneacetone) dipalladium(0) (24 mg, 0.026 mmol), 9,9-dimethyl-4,5-bis(diphenylphosphino)xanthene (22 mg, 0.039 mmol), sodium tert-butoxide (74.8 mg, 0.779 mmol), and the resulting mixture is heated to 70° C. for 12 h. The crude product is purified by preparative HPLC.

### Example 11

### Synthesis of 4-(2,3-dihydro-1,4-benzodioxin-6-yl)-7-methoxy-6-(2-methoxyethoxy)cinnoline

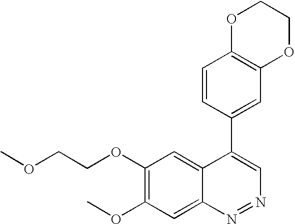

Into a 5 mL microwave tube is added 4-bromo-7-methoxy-6-(2-methoxyethoxy)-cinnoline (0.187 mmol), 1,4-benzodioxane-6-boronic acid (38.6 mg, 0.214 mmol), bis(triphenylphosphine)-palladium(II) chloride (26.2 mg, 0.0373 mmol), sodium carbonate (2.00 M solution in water, 140 μL) and a mixture of 1,2-dimethoxyethane:water:ethanol (7:3:2 ratio, 900 μL). The resulting suspension is subjected to microwave radiation at a temperature of 140° C. for 5.0 minutes. The mixture is then filtered through celite, which is washed with ethyl acetate. The organics were combined and washed with water, and then washed with brine. The organic layer is loaded onto an SCX column and the title compound is eluted.

### Example 12

### Synthesis of 7-methoxy-6-(2-methoxyethoxy)-4-(2-phenylmorpholin-4-yl)cinnoline

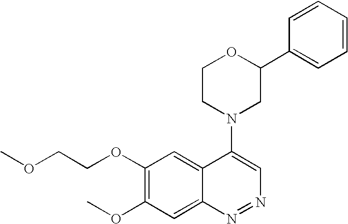

Into a 10 ml sealed microwave tube is added 4-bromo-7-methoxy-6-(2-methoxyethoxy)cinnoline (0.371 mmol), 2-phenylmorpholine hydrochloride (89.6 mg, 0.449 mmol), tris(dibenzylideneacetone)dipalladium(0) (20.4 mg, 0.0223 mmol), 9,9-dimethyl-4,5-bis(diphenylphosphino)xanthane (22.8 mg, 0.0394 mmol), sodium tert-butoxide (93.8 mg, 0.976 mmol) and toluene (3 mL). The resulting suspension is stirred at 50° C., and then filtered through celite, which is washed with ethyl acetate. The combined organics are concentrated, and the crude product is purified by column chromatography.

### Example 13

### Synthesis of 7-methoxy-6-(2-methoxyethoxy)-4-(3-phenylpyrrolidin-1-yl)cinnoline

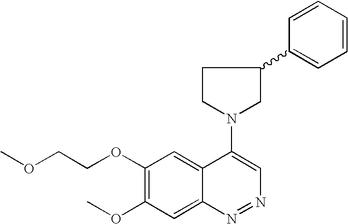

Into a 5 mL microwave tube is added 4-bromo-7-methoxy-6-(2-methoxyethoxy)cinnoline (0.4381 mmol) and 3-phenylpyrrolidine (52.9 mg, 0.359 mmol), tris(dibenzylideneacetone)-dipalladium(0) (17.4 mg, 0.0190 mmol), 9,9-dimethyl-4,5-bis(diphenylphosphino)xanthene (31.5 mg, 0.0544 mmol), sodium tert-butoxide (74.3 mg, 0.773 mmol) and toluene (0.7 mL). The resulting suspension is stirred at 60° C. overnight. Aqueous hydrogen chloride (0.1 M, 5 mL) is then added. The solution is filtered through celite, and the solution is adjusted to a pH of approximately 11-12. The product is extracted with ethyl acetate and the organics are washed with an aqueous saturated solution of sodium bicarbonate. The organic layer is dried over sodium sulfate, filtered, and concentrated in vacuo. The crude product is purified.

### Example 14

### Synthesis of 4-[5-(benzyloxy)-1H-indazol-1-yl]-7-methoxy-6-(2-methoxyethoxy)cinnoline

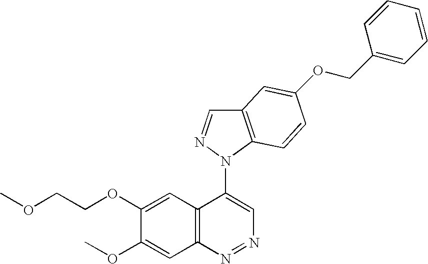

Into a 10 ml sealed microwave tube is added 4-bromo-7-methoxy-6-(2-methoxyethoxy)cinnoline (0.929 mmol), 5-(benzyloxy)-1H-indazole (189 mg, 0.844 mmol), toluene (5.0 mL), tris(dibenzylideneacetone) dipalladium(0) (40 mg, 0.04 mmol) 9,9-dimethyl-4,5-bis(diphenylphosphino)xanthene (49 mg, 0.084 mmol), and sodium tert-butoxide (240 mg, 2.5 mmol) and the reaction is heated to at 80° C. The crude product is purified.

### Example 15

### Synthesis of 7-methoxy-6-(2-methoxyethoxy)-4-(5-pyridin-4-yl-1H-indazol-1-yl)cinnoline

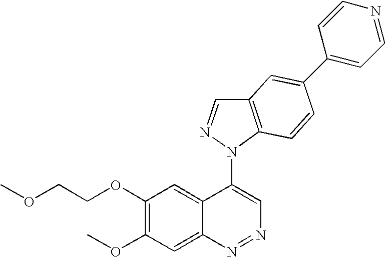

Into a 5 mL microwave tube is added 4-bromo-7-methoxy-6-(2-methoxyethoxy)cinnoline (0.856 mmol), 5-pyridin-4-yl-1H-indazole (200 mg, 1.02 mmol), copper(I) iodide (33 mg, 0.17 mmol), potassium carbonate (238.1 mg, 1.723 mmol), N,N′-dimethyl-1,2-ethanediamine (36 μL, 0.34 mmol) and toluene (6.91 mL). The suspension is heated at 115° C. for 24 hours. The material is diluted in 100 mL of 5% MeOH in DCM and filtered through a pad a celite and washed with DCM. The filtrate is collected and purified.

### Example 16

### Synthesis of 4-(3-benzylpyrrolidin-1-yl)-7-methoxy-6-(2-methoxyethoxy)cinnoline

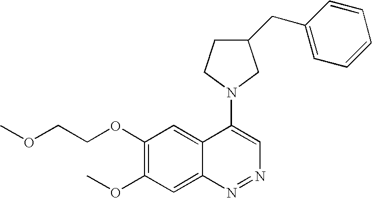

Into a 10 mL sealed microwave tube is added 4-bromo-7-methoxy-6-(2-methoxy-ethoxy)cinnoline (0.186 mmol), 3-benzylpyrrolidine (36.0 mg, 0.223 mmol), toluene (1.5 mL, 0.014 mol), tris(dibenzylideneacetone)dipalladium(0) (8.0 mg, 0.0087 mmol), 9,9-dimethyl-4,5-bis(diphenylphosphino)xanthene (11 mg, 0.019 mol) and sodium tert-butoxide (26.8 mg, 0.279 mol). The reaction mixture is heated to 50° C. and then is loaded onto a SCX column and pushed through with MeOH (1 volume). Elution with NH₃ in MeOH, followed by concentration on the rotovap provides the crude product, which is purified by chromatography.

### Example 17

### Synthesis of 4-[2-(4-fluorophenyl)-2-methylmorpholin-4-yl]-7-methoxy-6-(2-methoxyethoxy)cinnoline

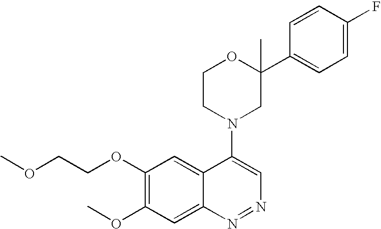

Into a flame-dried 5 mL microwave tube under argon is added 4-bromo-7-methoxy-6-(2-methoxyethoxy)cinnoline (0.312 mmol), commercially available 2-(4-fluorophenyl)-2-methyl-morpholine (49.9 mg, 0.256 mol), tris(dibenzylideneacetone)-dipalladium(0) (12.1 mg, 0.0132 mmol), 9,9-dimethyl-4,5-bis(diphenylphosphino)xanthene (15.0 mg, 0.0259 mmol), sodium tert-butoxide (35.7 mg, 0.371 mmol) and toluene (0.6 mL, 6 mmol). The resulting suspension is stirred at 50° C. overnight. The product is purified.

### Example 18

### Synthesis of 4-{4-[4-(cyclopropylmethyl)piperazin-1-yl]-1H-indazol-1-yl}-7-methoxy-6-(2-methoxyethoxy)cinnoline

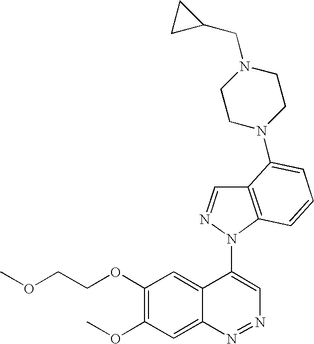

Step 1. A solution of 4-bromo-1H-indazole (0.197 g, 1.00 mmol) in 3 mL of DMA is stirred with n-butyl lithium (0.0704 g, 1.10 mmol) at −30° C. for 30 minutes. A mixture of tris(dibenzylideneacetone)dipalladium(0), 4-bromo-7-methoxy-6-(2-methoxyethoxy)cinnoline (1.00 mmol) and triethylamine (420 uL, 3.0 mmol) in 3 mL of DMA is added and the temperature of the reaction mixture is raised to 25° C. for 5 minutes and then to 85° C. for 12 hours. The reaction is monitored by LC/MS. Upon completion, the solvent is evaporated and the residue is diluted with 10% MeOH/DCM and filtered through celite. The solution is concentrated and purified by silica gel chromatography to give 4-(4-bromo-1H-indazol-1-yl)-7-methoxy-6-(2-methoxyethoxy)cinnoline.

Step 2. A mixture of 4-(4-bromo-1H-indazol-1-yl)-7-methoxy-6-(2-methoxyethoxy)cinnoline (0.000519 mol), piperazine (0.4 g, 0.005 mol), tetrahydrofuran (6.00 mL, 0.0740 mol), 2-dicyclohexyl-phosphino-2′,4′,6′-tri-1-propyl-1,1′-biphenyl (0.035 g, 0.073 mmol), tris(dibenzylideneacetone)-dipalladium(0) (0.035 g, 0.038 mmol) and sodium tert-butoxide (0.150 g, 0.00156 mol) is microwaved at 140° C. The resulting mixture is diluted with 5% MeOH/DCM and filtered through a pad of celite. The solution is concentrated and purified by column chromatography to give 7-methoxy-6-(2-methoxyethoxy)-4-(4-piperazin-1-yl-1H-indazol-1-yl)-cinnoline.

Step 3. 7-Methoxy-6-(2-methoxyethoxy)-4-piperazin-1-yl-1H-indazol-1-yl)cinnoline (0.051 mmol), cyclopropylmethyl bromide (0.010 mL, 0.1 mmol), potassium carbonate (21.2 mg, 0.154 mmol) and DMA (2.0 mL) is combined and the reaction mixture is warmed to 80° C. for 3 hours. The solvent is evaporated and the residue is diluted with DCM and filtered through celite. The filtrate is concentrated and purified by silica gel chromatography.

### Example 19

### Synthesis of 7-methoxy-6-(2-methoxyethoxy)-4-(4-pyrrolidin-1-yl-1H-indazol-1-yl)cinnoline

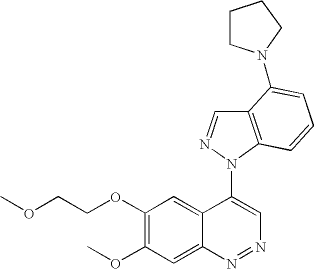

4-(4-Bromo-1H-indazol-1-yl)-6,7-dimethoxycinnoline (0.1.2 mmol), pyrrolidine (33 mg, 0.47 mmol), tetrahydrofuran (4.0 mL), 2-dicyclohexylphosphio-2′,4′,6′-tri-1-propyl-1,1′-biphenyl (8.0 mg, 0.017 mmol), sodium tert-butoxide (44.9 mg, 0.47 mmol) and tris-(dibenzylidene-acetone)dipalladium(0) (8 mg, 0.09 mmol) are combined in a microwave tube and irradiated in a microwave oven at 300 W to 140° C. for 8.30 minutes. The resulting mixture is diluted with DCM, filtered through celite, concentrated and purified by column chromatography.

### Example 20

### Synthesis of 4-(7-methoxy-6-(2-methoxyethoxy)cinnolin-4-yl)-6-(3-methoxyphenyl)morpholin-3-one

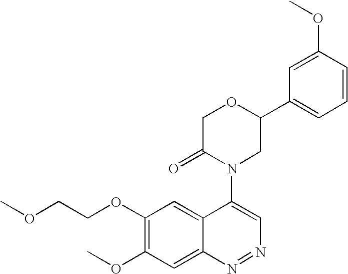

Into a 5 mL microwave tube is added 4-bromo-7-methoxy-6-(2-methoxyethoxy)cinnoline (0.236 mmol), 6-(3-methoxyphenyl)morpholin-3-one (41.7 mg, 0.201 mmol), copper(I) iodide (5.7 mg, 0.030 mmol), potassium carbonate (68.8 mg, 0.498 mmol), N,N′-dimethyl-1,2-ethanediamine (10 μL, 0.1 mmol) and tetrahydrofuran (0.3 mL, 0.004 mol). The reaction mixture is heated at 115° C., filtered through celite rinsing with methylene chloride and concentrated (rotovap). The crude product is purified.

### Example 21

### Synthesis of 1-{[1-(7-methoxy-6-(2-methoxyethoxy)cinnolin-4-yl)piperidin-3-yl]methyl}pyrrolidin-2-one

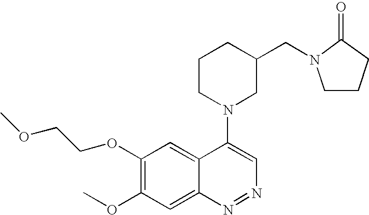

Into a flame-dried 5 mL microwave tube under argon is added 4-bromo-7-methoxy-6-(2-methoxyethoxy)cinnoline (0.369 mmol), 1-(piperidin-3-ylmethyl)pyrrolidin-2-one (50.7 mg, 0.278 mmol), tris(dibenzylideneacetone)dipalladium(0) (13.9 mg, 0.0152 mmol), 9,9-dimethyl-4,5-bis(diphenylphosphino)xanthene (17.4 mg, 0.0301 mmol), sodium tert-butoxide (41.1 mg, 0.428 mmol) and toluene (0.7 mL, 0.006 mol). The resulting suspension is warmed to 50° C. with stirring, cooled to room temperature and filtered through celite rinsing with 10% MeOH in DCM. The reaction mixture is concentrated and purified.

### Example 22

### Synthesis of 7-methoxy-6-(2-methoxyethoxy)-4-[2-(4-methoxyphenyl)-3-methylmorpholin-4-yl]cinnoline

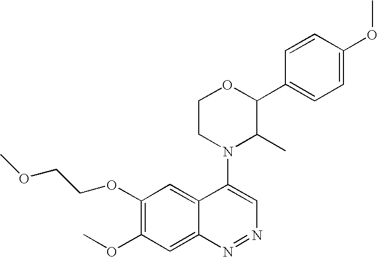

Into a 25 mL round bottom flask is added 4-bromo-7-methoxy-6-(2-methoxyethoxy)cinnoline (0.56 mmol), 2-(4-methoxyphenyl)-3-methylmorpholine (140 mg, 0.67 mmol), tris(dibenzylideneacetone)dipalladium(0) (26 mg, 0.028 mmol), 9,9-dimethyl-4,5-bis(diphenylphosphino)xanthene (32 mg, 0.056 mmol), sodium tert-butoxide (80 mg, 0.84 mol) and toluene (2 mL). The suspension is stirred at 55° C., and then flushed through an SCX column with methanol and eluted with 2.0 M ammonia in methanol. The material is purified.

### Example 23

### Synthesis of 1-(7-methoxy-6-(2-methoxyethoxy)cinnolin-4-yl)-N-(4-methoxybenzyl)piperidin-4-amine

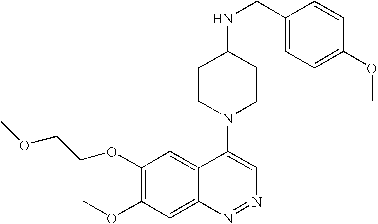

1-(6,7-Dimethoxycinnolin-4-yl)piperidin-4-amine (0.42 mmol), 2 mL of methylene chloride and 4-methoxybenzaldehyde (0.085 g, 0.62 mmol) are combined and stirred at room temperature for 30 minutes followed by the addition of sodium cyanoborohydride (0.08 g, 1 mmol). The resulting mixture is stirred overnight and the product is purified.

### Example 24

### Synthesis of N-[3-(7-methoxy-6-(2-methoxyethoxy)cinnolin-4-yl)phenyl]acetamide

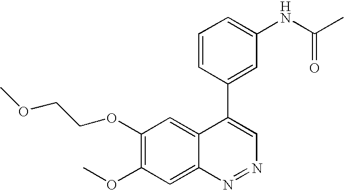

Into a microwave tube is added 4-bromo-7-methoxy-6-(2-methoxyethoxy)cinnoline (0.8 mmol), [3-(acetylamino)phenyl]boronic acid (100 mg, 0.8 mol), bis(triphenylphosphine) palladium(II) chloride (95.6 mg, 0.136 mmol), aqueous sodium carbonate (2.00 M, 0.28 mL) and a mixture of dimethoxyethane:water:ethanol (5 mL, 7:3:2). The resulting suspension is subjected to microwave radiation at 140° C. for 10 min. The reaction is filtered through celite, which is washed with methanol. The crude product is concentrated and purified by chromatography.

### Example 25

### Synthesis of 7-methoxy-6-(2-methoxyethoxy)-4-[3-(1-methyl-1H-pyrazol-4-yl)phenyl]cinnoline

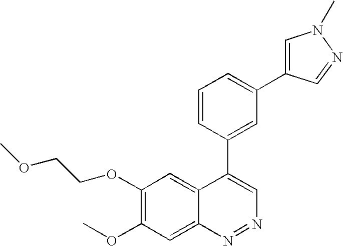

Step 1. 4-Bromo-7-methoxy-6-(2-methoxyethoxy)cinnoline (0.8 mmol), bis(triphenylphosphine)-palladium(II) chloride (95.6 mg, 0.136 mmol), aqueous sodium carbonate (2.00 M, 0.28 mL), 3-bromophenyl boronic acid (200 mg, 0.8 mol) and a mixture of 1,2-dimethoxyethane:water:ethanol (5 mL, 7:3:2) are added to a microwave tube and sealed. The resulting suspension is subjected to microwave radiation at 140° C. for 10 minutes. The reaction contents are filtered through celite, which is washed with methanol and dichloromethane and the organics are concentrated and purified to give 4-(3-bromophenyl)-7-methoxy-6-(2-methoxyethoxy)cinnoline.

Step 2. 4-(3-Bromophenyl)-7-methoxy-6-(2-methoxyethoxy)cinnoline (0.1 mmol), bis(triphenylphosphine)-palladium(II) chloride (17.8 mg, 0.0253 mmol), 1-methyl-4-(4,4,5,5-tetramethyl-1,3,2-dioxaborolan-1H-pyrazole (30 mg, 0.1 mmol), 2.00 M of sodium carbonate in water (0.052 mL) and a mixture of 1,2-dimethoxyethane:water:ethanol (0.9 mL, 7:3:2) are added to a microwave tube and sealed and irradiated in a microwave reactor. The reaction contents are filtered through celite, which is washed with methanol and dichloromethane and the organics are concentrated and purified.

### Example 26

### Synthesis of 7-methoxy-6-(2-methoxyethoxy)-4-(1-phenyl-1H-pyrazol-4-yl)cinnoline

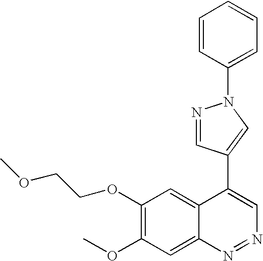

A mixture of 7-methoxy-6-(2-methoxyethoxy)-4-(1H-pyrazol-4-yl)cinnoline (0.2 mmol), phenylboronic acid (35.7 mg, 0.293 mmol), cupric acetate (35.5 mg, 0.196 mmol), triethylamine (0.134 mL, 0.965 mmol), pyridine (0.128 mL) and 1,4-dioxane (1.55 mL) is stirred at room temperature for 40 h. Water (15 mL) and ethyl acetate (25 mL) are added, and the mixture is filtered through celite. The organic layer is separated, washed with brine, dried (sodium sulfate), and concentrated in vacuo. The residue is purified by preparative HPLC.

### Example 27

### Synthesis of 2-(7-methoxy-6-(2-methoxyethoxy)cinnolin-4-yl)-6-piperidin-1-yl-3,4-dihydroisoquinolin-1(2H)-one

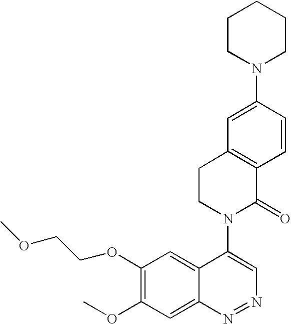

4-Bromo-7-methoxy-6-(2-methoxyethoxy)cinnoline (0.4746 mmol), 6-piperidin-1-yl-3,4-dihydroisoquinolin-1(2H)-one (130.8 mg, 0.5679 mmol), copper(I) iodide (8.4 mg, 0.044 mmol), potassium carbonate (132.0 mg, 0.9551 mmol), N,N′-dimethyl-1,2-ethanediamine (20 μL) and toluene (0.6 mL) are added to a 5 mL microwave tube, and the resulting suspension is heated at 115° C. The reaction is filtered thru celite, which is washed with ethyl acetate. The compound is purified.

### Example 28

### Synthesis of N-cyclopropyl-6-(7-methoxy-6-(2-methoxyethoxy)cinnolin-4-yl)-1H-indazole-3-carboxamide

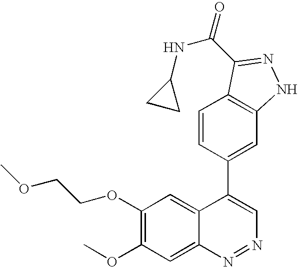

Step 1. Into a 10 ml microwave tube is added 4-bromo-7-methoxy-6-(2-methoxyethoxy)cinnoline (0.56 mmol), bis(triphenylphosphine)palladium(II) chloride (58.7 mg, 0.0836 mmol), ethyl 6-(4,4,5,5-tetramethyl-1,3,2-dioxaborolan-2-yl)-1H-indazole-3-carboxylate (260 mg, 0.84 mmol), aqueous sodium carbonate (2.00 M, 0.40 mL) and a mixture of dimethoxyethane:water:ethanol (50 mL, 7:3:2). The resulting mixture is subjected to microwave radiation at 140° C. for 5.0 minutes. A 20% mixture of methanol/dichloromethane (50 mL) is added, and the solution is filtered over celite and concentrated. Column chromatography purification affords ethyl 6-(7-methoxy-6-(2-methoxyethoxy)cinnolin-4-yl)-1H-indazole-3-carboxylate.

Step 2. A solution of potassium hydroxide in 85% methanol/water (2 M, 9 mL) is added to ethyl 6-(7-methoxy-6-(2-methoxyethoxy)cinolin-4-yl)-1H-indazole-3-carboxylate (0.33 mmol) and the resulting mixture is stirred at room temperature for 12 h, then at 60° C. for 3 h. The pH of the mixture is adjusted to ˜3 using trifluoroacetic acid, and the solvent is removed in vacuo. The residue is diluted with methanol/dichloromethane (20%, 30 mL) and stirred for 1 hour resulting in the formation of two layers. The lower layer is separated and the organics are combined and concentrated. The resulting residue is purified twice by column chromatography to afford 6-(7-methoxy-6-(2-methoxyethoxy)cinolin-4-yl)-1H-indazole-3-carboxylic acid.

Step 3. A mixture of 6-(7-methoxy-6-(2-methoxyethoxy)cinnolin-4-yl)-H-indazole-3-carboxylic acid (0.0856 mmol), cyclopropylamine (0.012 mL, 0.17 mmol), N,N′-diisopropylcarbodiimide (21 μL), 1-hydroxybenzotriazole (6 mg, 0.04 mol), and N,N-dimethylformamide (4.0 mL) is stirred at room temperature for 18 hours. The solvent is evaporated and the residue is dissolved in ethyl acetate (50 mL) and washed with aqueous sodium bicarbonate. The organic layer is concentrated and the product purified by column chromatography.

### Example 29

### Synthesis of N-cyclopropyl-5-(7-methoxy-6-(2-methoxyethoxy)cinnolin-4-yl)-4,5,6,7-tetrahydro-1H-pyrazolo[4,3-c]pyridine-3-carboxamide

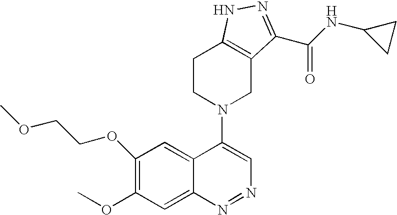

Step 1. To a solution of N,N-diisopropylamine (2.4 mL, 0.017 mol) in 20 mL of THF (20.0 mL, 0.246 mol) at 0° C. is added 2.0 M nBuLi in pentanes (8.5 mL). The reaction is stirred for 30 minutes at 0° C. and then cooled to −78° C. and a solution of 1-BOC-4-piperidone (3.20 g, 0.016 mol) in 20 mL of THF (20.0 mL, 0.246 mol) is added slowly. The mixture is stirred for 30 minutes at −78° C. and then a solution of diethyl oxalate (2.48 g, 0.017 mol) in THF (10.0 mL) is added in one portion. The mixture is stirred over night at room temperature. Water (200 mL) is added and the mixture is neutralized with 1 N HCl and extracted with 2×200 mL of EtOAc. The organic phase separated and washed with brine, dried (MgSO₄), filtered and concentrated under reduced pressure to provide crude tert-butyl 3-[ethoxy(oxo)acetyl]-4-oxopiperidine-1-carboxylate.

Step 2. A mixture of tert-butyl 3-[ethoxy(oxo)acetyl]-4-oxopiperidine-1-carboxylate (4.0 g, 0.013 mol) and acetic acid (8.0 mL, 0.141 mol) is treated drop-wise with hydrazine (1.0 mL, 0.032 mol) with stirring (note heat evolution). The mixture is stirred over night at room temperature and poured into an ice-cold saturated solution of NaHCO₃. The mixture is diluted with 50 mL of water and 50 mL of EtOAc. The organic fraction is washed with brine (25 mL), dried (MgSO₄) and concentrated to provide crude 5-tert-butyl 3-ethyl 1,4,6,7-tetrahydro-5H-pyrazolo[4,3-c]pyridine-3,5-dicarboxylate.

Step 3. A solution of 5-tert-butyl 3-ethyl 1,4,6,7-tetrahydro-5H-pyrazolo[4,3-c]pyridine-3,5-dicarboxylate (0.90 g, 0.0031 mol) in ethanol (30.0 mL) is treated with 5.0 M aqueous NaOH solution (10 mL). The reaction is stirred overnight at room temperature, diluted with 100 mL of water and washed with EtOAc. The aqueous fraction is acidified with 1.0 N aqueous HCL and extracted with EtOAc. The combined EtOAc extracts are washed with brine (25 mL), dried (MgSO₄) and concentrated to yield 5-(tert-butoxycarbonyl) 4,5,6,7-tetrahydro-1H-pyrazolo[4,3-c]pyridine-3-carboxylic acid as a white solid.

Step 4. 5-(tert-Butoxycarbonyl)-4,5,6,7-tetrahydro-1H-pyrazolo[4,3-c]pyridine-3-carboxylic acid (40 mg, 0.15 mmol), cyclopropylamine (21 μL, 0.3 mmol), N,N′-diisopropylcarbodiimide (30 μL, 0.19 mmol), 1-hydroxybenzotriazole (10 mg, 0.07 mmol), N,N-dimethylformamide (0.3 mL) and methylene chloride (3.0 mL) are combined and stirred at room temperature for 5 h. The mixture is then concentrated and the residue is taken up in 50 mL of EtOAc, washed with NaHCO₃ and concentrated. The residue is purified by silica gel chromatography using a gradient elution going from 1% MeOH in 1:1 hexane:EtOAc to 3% MeOH in 1:1 hexanes:EtOAc to provide tert-butyl 3-[(cyclopropylamino)carbonyl]-1,4,6,7-tetrahydro-5H-pyrazolo[4,3-c]pyridine-5-carboxylate as a white solid.

Step 5. tert-Butyl 3-[(cyclopropylamino)carbonyl]-1,4,6,7-tetrahydro-5H-pyrazolo[4,3-c]pyridine-5-carboxylate (0.034 g, 0.11 mmol), methylene chloride (2.0 mL) and trifluoroacetic acid (1.0 mL) are combined and stirred for 4 h at room temperature. The solvent is removed in vacuo and the residue is purified by trituration with ether to provide N-cyclopropyl-4,5,6,7-tetrahydro-1H-pyrazolo[4,3-c]pyridine-3-carboxamide trifluoroacetate salt as a white solid.

Step 6. A mixture of 4-bromo-7-methoxy-6-(2-methoxyethoxy)cinnoline (0.037 mmol), N-cyclopropyl-4,5,6,7-tetrahydro-1H-pyrazolo[4,3-c]pyridine-3-carboxamide trifluoroacetate (0.014 g, 0.046 mol), tris(dibenzylideneacetone)dipalladium(0) (3 mg, 0.004 mmol), N,N-dimethylacetamide (0.62 mL) and triethylamine (0.019 g, 0.18 mmol) is heated at 85° C. The solvent is removed in vacuo, and the residue is diluted with methanol/dichloromethane and then filtered. The solution is washed with aqueous sodium bicarbonate. The organics are concentrated, and the residue is purified.

### Example 30

### Synthesis of 4-[1-(4-fluorobenzyl)-1H-pyrazol-4-yl]-7-methoxy-6-(2-methoxyethoxy)cinnoline

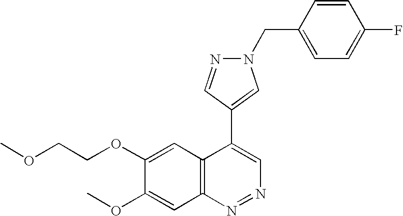

Step 1. Into a microwave tube is added 4-bromo-7-methoxy-6-(2-methoxyethoxy)cinnoline (0.8 mmol), bis(triphenylphosphine) palladium(II) chloride (95.6 mg, 0.136 mmol), tert-butyl-4,-(4,4,5,5-tetramethyl-1,3,2-dioxaborolan-2-yl)-1H-pyrazole-1-carboxylate (200 mg, 0.0008 mol), aqueous sodium carbonate (2.00 M, 0.28 mL) and a mixture of dimethoxyethane:water:ethanol (5 mL, 7:3:2). The resulting suspension is subjected to microwave radiation at 140° C. for 10 min. The reaction is filtered through celite, which is washed with methanol. Concentration, followed by chromatographic purification to give 7-methoxy-6-(2-methoxyethoxy)-4-(1H-pyrazol-4-yl)cinnoline.

Step 2. Sodium hydride (5 mg, 0.2 mmol) is added to dimethylformamide (2 mL) in a flame-dried round bottom flask under an atmosphere of nitrogen. 7-Methoxy-6-(2-methoxyethoxy)-4-(1H-pyrazol-4-yl)cinnoline (0.098 mmol) is added and the reaction stirred at room temperature for 1 h. A solution of α-bromo-4-fluorotoluene (60 mg, 0.0003 mol) in dimethylformamide (0.5 mL) (prepared under a nitrogen atmosphere) is then added, and the resulting mixture is stirred at room temperature for 16 h. The mixture is concentrated, and the residue purified.

### Example 31

### Synthesis of 7-methoxy-6-(2-methoxyethoxy)-4-[2-(4-methylpiperazin-1-yl)pyrimidin-5-yl]cinnoline

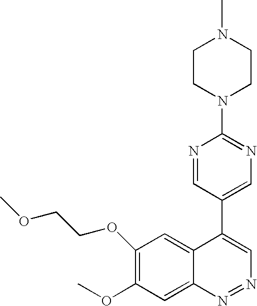

Into a 5 mL microwave tube is added 4-bromo-7-methoxy-6-(2-methoxyethoxy)cinnoline (0.186 mmol), 2-(4-methylpiperazin-1-yl)-5-(4,4,5,5-tetramethyl-1,3,2-dioxaborolan-2-yl)pyrimidine (145 mg, 0.478 mmol), bis(triphenylphosphine)palladium(II) chloride (26.9 mg, 0.0384 mmol), 2.00 M sodium carbonate in water (139 uL) and DME:Water:EtOH=7:3:2 (7:3:2,1,2-Dimethoxyethane:Water:Ethanol, 895 uL). The suspension is irradiated in a microwave at 300 W to 140° C. for 5.0 minutes. The reaction mixture is filtered through a celite plug and washed with methanol. The solution is concentrated under reduced pressure and the remaining residue is purified.

### Example 32

### Synthesis of 5-(7-methoxy-6-(2-methoxyethoxy)cinnolin-4-yl)-N-(pyridin-3-ylmethyl)pyridin-2-amine trifluoroacetic acid salt

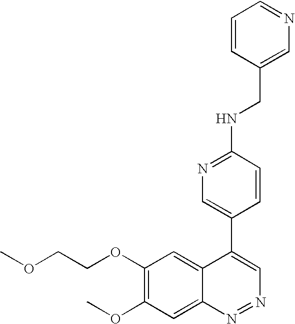

A mixture of 4-(6-fluoropyridin-3-yl)-6,7-dimethoxycinnoline (0.18 mmol), 3-(aminomethyl)pyridine (0.038 g, 0.35 mmol), and DMSO (1 mL) is heated in an oil bath at 120° C. for 16 h. The resulting solution is purified.

### Example 33

### Synthesis of 1-(4-(7-methoxy-6-(2-methoxyethoxy)cinnolin-4-yl)benzyl)azetidine-3-carboxylic acid

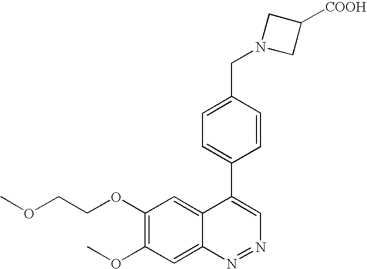

Step 1. A mixture of 4-bromo-7-methoxy-6-(2-methoxyethoxy)cinnoline (0.4 mmol), 4-formylphenylboronic acid (0.06 g, 0.4 mmol), palladium tetrakis-triphenylphosphine (0.02 g, 0.02 mmol), cesium carbonate (0.3 g, 1 mmol), and water (2 mL) is prepared in a sealed tube under nitrogen atmosphere and heated overnight at 80° C. The reaction mixture is allowed to cool to room temperature and concentrated to give 4-(7-methoxy-6-(2-methoxyethoxy)cinnolin-4-yl)benzaldehyde.

Step 2. To a solution of 4-(6,7-dimethoxycinnolin-4-yl)benzaldehyde (0.4 mmol) and 3-azetidinecarboxylic acid (0.04 g, 0.4 mmol) in dichloromethane is added sodium triacetoxyborohydride (0.1 g, 0.5 mmol) and trifluoroacetic acid (0.05 g, 0.4 mmol) at room temperature. More sodium triacetoxyborohydride is added and stirring is continued for another few hours until LC/MS shows full conversion. The product is purified.

### Example 34

### Synthesis of 7-methoxy-6-(2-methoxyethoxy)-4-(6-(1,2,3,6-tetrahydropyridin-4-yl)pyridin-3-yl)cinnoline

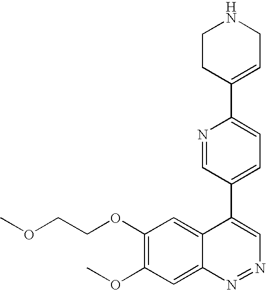

Step 1. Into a suspension of 4-bromo-7-methoxy-6-(2-methoxyethoxy)cinnoline (1.9 mmol), 2-chloropyridine-5-boronic acid (0.29 g, 1.9 mmol), and disodium carbonate monohydrate (0.35 mg, 2.8 mmol) in a mixed solvent of DME (3 mL), EtOH (1.8 mL) and water (1.5 mL) is bubbled N₂ for 5 min. Then dichlorobis(triphenylphosphine)palladium(II) (0.13 g, 0.19 mmol) is added and the reaction mixture is heated at 90° C. for 3 h. The reaction mixture is cooled to room temperature, diluted with EtOAc and water and the product is isolated by filtration. The solid collected is washed with a small amount of EtOAc and ether, dried in a vacuum oven to give 4-(6-chloropyridin-3-yl)-7-methoxy-6-(2-methoxyethoxy)cinnoline.

Step 2. A mixture of 4-(6-chloropyridin-3-yl)-7-methoxy-6-(2-methoxyethoxy)cinnoline (0.4 mmol), tert-butyl 4-(4,4,5,5-tetramethyl-1,3,2-dioxaborolan-2-yl)-5,6-dihydropyridine-1(2H)-carboxylate (0.18 g, 0.6 mmol), and tetrakis(triphenylphosphine)palladium (0.023 g, 0.02 mmol) in dioxane (1 mL) is treated with 2M aqueous solution of potassium carbonate (0.16 g, 1.2 mmol). The reaction mixture is heated at 100° C. for 2 h. After cooling to room temperature, the reaction mixture is diluted with EtOAc and saturated NH₄Cl and is then transferred to a separatory funnel. The layers are separated and the aqueous phase is extracted with EtOAc. The combined organics are washed with brine, dried over Na₂SO₄, filtered and concentrated. The crude product is chromatographed to provide tert-butyl 4-(5-(7-methoxy-6-(2-methoxyethoxy)cinnolin-4-yl)pyridin-2-yl)-5,6-dihydropyridine-1(2H)-carboxylate.

Step 3. To tert-butyl 4-(5-(7-methoxy-6-(2-methoxyethoxy)cinnolin-4-yl)pyridin-2-yl)-5,6-dihydropyridine-1(2H)-carboxylate (0.16 mmol) dissolved in DCM (1 mL) is added TFA (0.3 ml, 3.9 mmol). The reaction mixture is stirred at RT under nitrogen for 1 h. The solvent is removed in vacuo and the residue is partitioned between DCM and saturated NaHCO₃. The aqueous fraction is back extracted with DCM and the combined organics are dried (Na₂SO₄) and concentrated and the residue is purified.

### Example 35

### Synthesis of 4-(6-(cyclopropylmethoxy)pyridin-3-yl)-7-methoxy-6-(2-methoxyethoxy)cinnoline

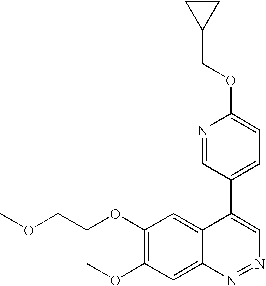

To the suspension of 4-bromo-7-methoxy-6-(2-methoxyethoxy)cinnoline (0.26 mmol), 2-(cyclopropylmethoxy)-5-(4,4,5,5-tetramethyl-1,3,2-dioxaborolan-2-yl)pyridine (75 mg, 0.27 mmol), and disodium carbonate monohydrate (48 mg, 0.39 mmol) in a mixed solvent of DME (0.5 mL), EtOH (0.3 mL) and water (0.25 mL) is bubbled N₂ for 5 min. Then dichlorobis(triphenylphosphine)palladium(II) (18 mg, 0.026 mmol) is added and the reaction mixture is heated at 90° C. for 2 h. The reaction mixture is cooled to room temperature, diluted with EtOAc and water, and transferred to a separatory funnel. The layers are separated and the aqueous is extracted with EtOAc. The combined organics are washed with brine, dried over Na₂SO₄, filtered and concentrated. The crude product is chromatographed for purification.

### Example 36

### Synthesis of 7-methoxy-6-(2-methoxyethoxy)-4-(4-(oxazol-2-yl)phenyl)cinnoline

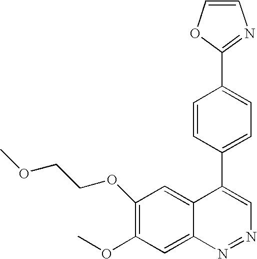

A mixture of 2-(4-(4,4,5,5-tetramethyl-1,3,2-dioxaborolan-2-yl)phenyl)oxazole (0.089 g, 0.33 mmol), 4-bromo-7-methoxy-6-(2-methoxyethoxy)cinnoline (0.3 mmol), palladium tetrakis-triphenylphosphine (0.017 g, 0.015 mmol), cesium carbonate (0.26 g, 0.80 mmol), and water (2.4 mL) are added to a sealed tube under atmosphere of N₂. The resulting mixture is heated to 80° C. The reaction mixture is filtered over a cake of celite and then rinsed with MeOH, and the residue purified.

### Example 37

### Synthesis of 6-(7-methoxy-6-(2-methoxyethoxy)cinnolin-4-yl)-N-isopropylbenzo[d]isothiazole-3-carboxamide

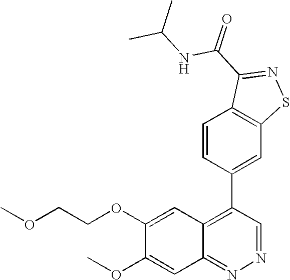

Step 1. A solution of 3-bromobenzenethiol (6.00 g, 31.7 mmol) in CH₂Cl₂ (16 mL) is added slowly dropwise to neat oxalyl chloride (13.8 mL, 159 mmol) at room temperature with stirring. The resultant mixture is heated to reflux and stirred overnight at which point LC/MS analysis is used to determine that the reaction is complete. The reaction mixture is then cooled to room temperature and the volatiles are removed in vacuo. A yellow solid is obtained which is S-3-bromophenyl-2-chloro-2-oxoethanethioate.

Step 2. Aluminum chloride (12.9 g, 96.6 mmol) is stirred at room temperature in carbon disulfide (10.8 ml) until all the solids are suspended. A suspension of S-3-bromophenyl-2-chloro-2-oxoethanethioate (6.00 g, 21.5 mmol) in carbon disulfide (10.8 mL, 2M) is then added very slowly dropwise to the AlCl₃ suspension. The flask is then equipped with a reflux condenser and the reaction mixture is heated to 45° C. for 2 hrs. LCMS analysis can be used to confirm complete consumption of the starting material. The reaction mixture is cooled to room temperature and the supernatant is poured into ice water. Water is then added to the solids remaining in the flask (Caution: very exothermic!) and diethyl ether is added. The resultant orange precipitate is poured into ice water and filtered to obtain an orange solid which is dried overnight to give 6-bromobenzo[b]thiophene-2,3-dione.

Step 3. Ammonium hydroxide (28% aqueous solution) (3.91 mL, 28.4 mmol) is added slowly dropwise to a solution of 6-bromobenzo[b]thiophene-2,3-dione (300 mg, 1.23 mmol) in MeOH (2 ml) cooled to 10° C., maintaining the temperature between 10-20° C. The ice bath is removed and the resultant mixture is stirred overnight at room temperature after which time the reaction mixture is re-cooled to 10° C. and hydrogen peroxide (30%) (0.391 mL, 3.83 mmol) is added slowly dropwise. The ice bath is removed and the reaction mixture is stirred at room temperature for 1 hour. The resulting precipitate is filtered and washed with water. After air-drying, a light tan solid is obtained which is 6-bromobenzo[d]isothiazole-3-carboxamide.

Step 4. A suspension of 6-bromobenzo[d]isothiazole-3-carboxamide (274 mg, 1066 μmol) in EtOH (5.9 mL) and 6N sodium hydroxide (356 μL, 2135 μmol) is heated to reflux for 2 hrs. LC/MS analysis can be used to confirm complete conversion to the acid. The reaction mixture is cooled to room temperature, acidified with 1N HCl, and extracted with ethyl acetate. The combined organics are washed with brine, dried over MgSO₄, filtered and concentrated to give 6-bromobenzo[d]isothiazole-3-carboxylic acid which is used without further purification.

Step 5. Sulfuryl dichloride (86.7 mg, 728 μmol) is added to a solution of 6-bromobenzo[d]-isothiazole-3-carboxylic acid (188 mg, 728 μmol). The reaction mixture is stirred for 30 min before removing the volatiles by rotovap. The residue is taken up in CH₂Cl₂ (0.587 ml) and a solution of 2-propylamine (62.5 μL, 728 μmol) and triethylamine (101 μl, 728 μmol) in CH₂Cl₂ (1.2 ml) is added. The reaction mixture is stirred at room temperature until LC/MS analysis indicates complete conversion to the desired product. The reaction mixture is diluted with distilled water and ethyl acetate. The layers are separated and the aqueous is extracted with ethyl acetate. The combined organics are washed with brine and dried over Na₂SO₄, filtered and concentrated to give 6-bromo-N-isopropylbenzo[d]isothiazole-3-carboxamide.

Step 6. A solution of 6-bromo-N-isopropylbenzo[d]isothiazole-3-carboxamide (200 mg, 668 μmol), 4,4,5,5-tetramethyl-2-(4,4,5,5-tetramethyl-1,3,2-dioxaborolan-2-yl)-1,3,2-dioxaborolane (204 mg, 802 μmol), potassium acetate (131 mg, 1337 μmol), and dichloropalladiumbis-(diphenylphosphinoferrocene) (34 mg, 47 μmol) in dioxane (3.2 mL) is heated to 130 C overnight after which time LC/MS analysis indicates complete conversion to the desired product. The reaction mixture is filtered through celite give a brown solid. Purification is performed by Biotage pre-packed silica gel column (25M) using a gradient of 12-100% ethyl acetate/hexanes to give N-isopropyl-6-(4,4,5,5-tetramethyl-1,3,2-dioxaborolan-2-yl)benzo[d]isothiazole-3-carboxamide.

Step 7. To a solution of N-isopropyl-6-(4,4,5,5-tetramethyl-1,3,2-dioxaborolan-2-yl)benzo[d]-isothiazole-3-carboxamide (69 mg, 199 μmol) in DME (2.4 mL) is added 4-bromo-7-methoxy-6-(2-methoxyethoxy)cinnoline (199 μmol), bis(triphenylphosphine)palladium (II) chloride (7.0 mg, 10.0 μmol) followed by an aqueous solution of cesium carbonate (175 mg, 538 μmol) (1 ml H₂0). The reaction mixture is heated to 80° C. The reaction mixture is cooled to room temperature, diluted with distilled water and ethyl acetate. The layers are separated and the aqueous is extracted with ethyl acetate. The combined organics are washed with brine, dried over Na₂SO₄, filtered and concentrated. The residue is purified.

### Example 38

### Synthesis of 4-(6-(3,3-difluoroazetidin-1-yl)pyridin-3-yl)-7-methoxy-6-(2-methoxyethoxy)cinnoline

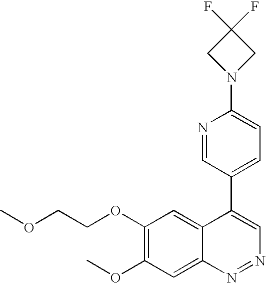

Step 1. To a 250 mL round-bottomed flask is added 4-bromo-7-methoxy-6-(2-methoxyethoxy)cinnoline (14.89 mmol) and tetrakis(triphenylphosphine)palladium (0) (0.8667 g, 0.7444 mmol) in 250 mL 1,2-dimethoxyethane. 6-Fluoropyridin-3-ylboronic acid (0.2849 g, 1.983 mmol) is added, followed by an aqueous solution of cesium carbonate (1.6792 g, 4.868 mmol) (10 mL water), and the reaction mixture is stirred at 80° C. for 3 hours. The reaction mixture is allowed to cool to room temperature. The solution is placed in a separatory funnel and deionized water and ethyl acetate is added. The aqueous layer is extracted with ethyl acetate. The combined organic layers are washed with water, brine, dried with MgSO₄, filtered, and concentrated. The tan solid is taken up in ether and allowed to stir for 15 minutes. The solid is then filtered and dried by vacuum to produce 4-(6-fluoropyridin-3-yl)-7-methoxy-6-(2-methoxyethoxy)cinnoline.

Step 2. In a microwave vial is placed 4-(6-fluoropyridin-3-yl)-7-methoxy-6-(2-methoxyethoxy)cinnoline (0.218 mmol) and potassium carbonate (0.3126 g, 2.22 mmol) in 2 mL DMSO. 3,3-Difluoroazetidine hydrochloride (0.2799 g, 2.18 mmol) is added and the temperature is brought to 90° C. to stir overnight. The reaction solution is allowed to cool to room temperature. The solution is moved to a separatory funnel and deionized water and ethyl acetate is added. The aqueous layer is extracted with ethyl acetate. The combined organic layers are washed with water, brine, dried with MgSO₄, filtered, and concentrated.

### Example 39

### Synthesis of provide 4-(5-(7-methoxy-6-(2-methoxyethoxy)cinnolin-4-yl)pyridin-2-yl)-1-methylpiperazin-2-one

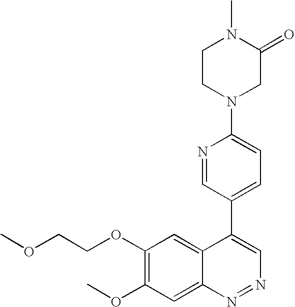

In a microwave vial is placed 4-(6-fluoropyridin-3-yl)-6,7-dimethoxycinnoline (0.229 mmol) in 2 ml DMSO. 1-Methylpiperazin-2-one hydrochloride (0.3626 g, 2.29 mmol) and potassium carbonate (0.147 ml, 2.40 mmol) is added and the temperature is brought to 90° C. to stir overnight. The reaction solution is allowed to cool to room temperature. The solution is moved to a separatory funnel and deionized water and ethyl acetate are added. The aqueous layer is extracted with ethyl acetate. The combined organic layers are washed with water, brine, dried with MgSO₄, filtered, and concentrated. The crude product is adsorbed onto a plug of silica gel and chromatographed.

### Example 40

### Synthesis of 7-methoxy-6-(2-methoxyethoxy)-4-(4-morpholin-4-yl-2,3-dihydro-1H-indol-1-yl)quinazoline

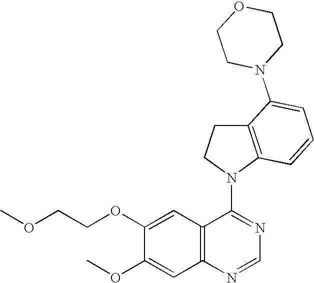

Step 1. 4-Bromoindole (5.00 mL, 0.0399 mol) is dissolved in a mixture of acetic acid (5.00 mL, 0.0879 mol) and methanol (25.0 mL, 0.617 mol) and cooled to 0° C. Sodium cyanoborohydride (7.52 g, 0.120 mol) is added and the mixture is slowly warmed to room temperature over a period of 1 h. The reaction mixture is then concentrated and neutralized using a saturated aqueous solution of sodium bicarbonate. The organics are extracted with ether and ethyl acetate and the combined organics are washed with brine, dried, filtered, and concentrated to afford 4-bromoindoline.

Step 2. 4-Bromo-7-methoxy-6-(2-methoxyethoxy)cinnoline (0.0099 mol) is added to a solution of 4-bromoindoline (1970 mg, 0.00995 mol) in N,N-dimethylacetamide (50 mL). Sodium iodide (700 mg, 0.004 mol) and potassium carbonate (550 mg, 0.0398 mol) are then added, and the resulting mixture is heated at 160° C. for 2.75 h. The reaction mixture is diluted with water and extracted with ethyl acetate. The organic layer is washed with water and brine, dried, filtered, and concentrated to afford 4-(4-bromo-2,3-dihydro-1H-indol-1-yl)-7-methoxy-6-(2-methoxyethoxy)quinazoline.

Step 3. 4-(4-bromo-2,3-dihydro-1H-indol-1-yl)-7-methoxy-6-(2-methoxyethoxy)quinazoline (0.0005 mol), morpholine (54.2 μL, 0.621 mmol) tetrahydrofuran (4.00 mL), tris(dibenzylideneacetone)-dipalladium(0) (20 mg, 0.02 mmol), 9,9-dimethyl-4,5-bis(diphenylphosphino)xanthane (30 mg, 0.052 mmol), sodium tert-butoxide (74.6 mg, 0.777 mmol) are added to a 10 ml sealed microwave tube and the resulting mixture is heated to 50° C. for 8 h. The mixture is purified.

### Example 41

### Synthesis of 7-methoxy-6-(2-methoxyethoxy)-4-(6-morpholin-4-ylpyridin-3-yl)cinnoline

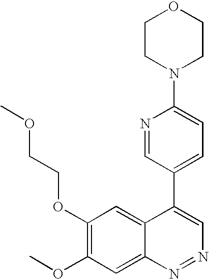

Into a 5 mL microwave tube was added 4-bromo-7-methoxy-6-(2-methoxyethoxy)cinnoline (58.6 mg, 0.187 mmol), 4-[5-(4,4,5,5-tetramethyl-[1,3,2]-dioxaborolan-2-yl)-pyridin-2-yl]-morpholine (140 mg, 0.482 mmol), bis(triphenylphosphine)palladium (II) chloride (27.1 mg, 0.039 mmol), 2.0 M Na₂CO₃ in water (140 μL) and 900 μL of a solution of DME:water:EtOH (7:3:2). The cloudy brown suspension was irradiated in a microwave reactor for 5.0 minutes at 140° C. and the material was filtered through a plug of Celite and rinsed with MeOH. The filtrate was concentrated and the product purified by rotary chromatography using a gradient going from 100% CHCl₃ to 10% MeOH/90% CHCl₃ to provide 70 mg (90% yield) of 7-methoxy-6-(2-methoxyethoxy)-4-(6-morpholin-4-ylpyridin-3-yl)cinnoline.

### Biological Examples

### Example 42

### mPDE10A7 Enzyme Activity and Inhibition

Enzyme Activity:

To analyze the enzyme activity, 5 μL of serial diluted mPDE10A7 containing lysate were incubated with equal volumes of diluted (100-fold) fluorescein labeled cAMP or cGMP for 30 minutes in MDC HE 96-well assay plates at room temperature. Both the enzyme and the substrates were diluted in the following assay buffer: Tris/HCl (pH 8.0) 50 mM, MgCl₂ 5 mM, 2-mercaptoethanol 4 mM, BSA 0.33 mg/mL. After incubation, the reaction was stopped by adding 20 μL of diluted (400-fold) binding reagents and was incubated for an hour at room temperature. The plates were counted in an Analyst GT (Molecular Devices) for fluorescence polarization. An IMAP Assay kit (Molecular Device) was used to assess enzyme properties of mmPDE10A7. Data were analyzed with SoftMax Pro.

Enzyme Inhibition:

To check the inhibition profile, 10 μL of serial diluted compounds were incubated with 30 μl of diluted PDE enzymes in a 96-well polystyrene assay plate for 30 minutes at room temperature. After incubation, 5 μL of the compound-enzyme mixture were aliquoted into a MDC HE black plate, mixed with 5 μl of 100-fold diluted fluorescein labeled substrates (cAMP or cGMP), and incubated for 30 minutes at room temperature. The reaction was stopped by adding 20 μL of diluted binding reagents and counted in an Analyst GT for fluorescence polarization. The data were analyzed with SoftMax Pro.

### Example 43

### Apomorphine Induced Deficits in Prepulse Inhibition of the Startle Response in Rats, an In Vivo Test for Antipsychotic Activity

The thought disorders that are characteristic of schizophrenia may result from an inability to filter, or gate, sensorimotor information. The ability to gate sensorimotor information can be tested in many animals as well as in humans. A test that is commonly used is the reversal of apomorphine-induced deficits in the prepulse inhibition of the startle response. The startle response is a reflex to a sudden intense stimulus such as a burst of noise. In this example, rats are exposed to a sudden burst of noise, at a level of 120 db for 40 msec, e.g. the reflex activity of the rats is measured. The reflex of the rats to the burst of noise may be attenuated by preceding the startle stimulus with a stimulus of lower intensity, at 3 to 12 db above background (65 db), which will attenuate the startle reflex by 20 to 80%.

The prepulse inhibition of the startle reflex, described above, may be attenuated by drugs that affect receptor signaling pathways in the CNS. One commonly used drug is the dopamine receptor agonist apomorphine. Administration of apomorphine will reduce the inhibition of the startle reflex produced by the prepulse. Antipsychotic drugs such as haloperidol will prevent apomorphine from reducing the prepulse inhibition of the startle reflex. This assay may be used to test the antipsychotic efficacy of PDE10 inhibitors, as they reduce the apormorphine-induced deficit in the prepulse inhibition of startle.

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

What is claimed:

1. A compound of Formula (I): 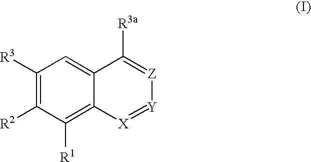
wherein:
Y and Z are nitrogen and X is —CR═ (where R is hydrogen, alkyl, halo, or cyano); or X and Y are nitrogen and Z is —CH═; or X and Z are nitrogen and Y is ═CH—;
one of R¹, R², and R³ is cycloalkyloxy, cycloalkylalkyloxy, hydroxyalkyl, hydroxyalkyloxy, alkoxyalkyl, alkoxyalkyloxy, -(alkylene)NR¹³R¹⁴ or —O-(alkylene)NR¹⁵R¹⁶ [(where R¹³, R¹⁴, R¹⁵, and R¹⁶ are independently hydrogen or alkyl) and wherein one or two carbon atoms in the alkyl chain in hydroxyalkyl, hydroxyalkyloxy, alkoxyalkyl, alkoxyalkyloxy, -(alkylene)NR¹³R¹⁴ or —O-(alkylene)NR¹⁵R¹⁶ are optionally replaced by one to two oxygen or nitrogen atom(s)] and the other two of R¹, R², and R³ are independently selected from hydrogen, alkyl, alkoxy, cycloalkyl, halo, haloalkyl, haloalkoxy, cyano, hydroxy, carboxy, alkoxycarbonyl, amino, alkylamino, dialkylamino, alkylcarbonyl, cycloalkyl, cycloalkyloxy, cycloalkylalkyloxy, hydroxyalkyl, hydroxyalkyloxy, alkoxyalkyl, alkoxyalkyloxy, -(alkylene)NR¹⁷R¹⁸ or —O-(alkylene)NR¹⁹R²⁰ [(where R¹⁷, R¹⁸, R¹⁹, and R²⁰ are independently hydrogen or alkyl and wherein one or two carbon atoms in the alkyl chain in hydroxyalkyl, alkoxyalkyl, -(alkylene)NR¹⁷R¹⁸ or —O-(alkylene)NR¹⁹R²⁰ are optionally replaced by one to two oxygen or nitrogen atom(s)]; and
R^{3a} is aryl, heteroaryl, or heterocyclyl ring substituted with:
R⁴ where R⁴ is hydrogen, alkyl, halo, haloalkyl, haloalkoxy, cycloalkyl, cycloalkylalkyl, aryl, heteroaryl, heterocyclyl, aralkyl, heteroaralkyl, heterocyclylalkyl, or —X¹R⁷ (where X¹ is —O—, —CO—, —C(O)O—, —OC(O)—, —NR⁸CO—, —CONR⁹—, —NR¹⁰—, —S—, —SO—, —SO₂—, —NR¹¹SO₂—, or —SO₂NR¹— where R⁸-R¹² are independently hydrogen, alkyl, hydroxyalkyl, alkoxyalkyl, aryl, aralkyl, heteroaryl, heteroaralkyl, acyl, or heterocyclylalkyl and R⁷ is cycloalkyl, cycloalkylalkyl, aryl, heteroaryl, heterocyclyl, aralkyl, heteroaralkyl, or heterocyclylalkyl); and
R⁵ and R⁶ where R⁵ and R⁶ are independently hydrogen, alkyl, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, acyl, aminocarbonyl, aminosulfinyl, aminosulfonyl, monosubstituted amino, disubstituted amino, aryl, heteroaryl or heterocyclyl;
and wherein the aromatic or alicyclic ring in R⁴, R⁵, R⁶, and R⁷ is optionally substituted with one to three substitutents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, cyano, nitro, carboxy, alkoxycarbonyl, alkylthio, sulfinyl, sulfonyl, aminocarbonyl, aminosulfinyl, aminosulfonyl, monosubstituted amino, disubstituted amino, optionally substituted phenyl, optionally substituted heteroaryl, or optionally substituted heterocyclyl; and additionally substituted with one or two substitutents independently selected from R^{d} and R^{e} where R^{d} and R^{e} are hydrogen or fluoro; or
an individual stereoisomer, a mixture of stereoisomers, or a pharmaceutically acceptable salt thereof, provided that:
(i) when X and Z are nitrogen, R¹ is hydrogen, R² is alkoxy, alkoxyalkyloxy (wherein one or two carbon atoms in alkoxyalkyloxy are optionally replaced by one to two oxygen atoms), hydroxyalkoxy, or —O-(alkylene)-NR¹³R¹⁴ where R¹³ and R¹⁴ are independently hydrogen or alkyl, and R³ is hydrogen, alkoxy, alkoxyalkyloxy, or hydroxyalkyloxy, then R^{3a} is not
2,3-dihydroindolyl, 2-oxoindolyl, indolyl, 7-aza-2-oxo-indol-3-yl, 4-aza-2-oxo-indol-3-yl, 5,7-diazaoxindol-3-yl, or piperidinyl, each of which is substituted with R⁴, R⁵ or R⁶ as defined above;
6-chloro-7-aza-2-oxo-indol-3-yl; 2-alkyl-5H-pyrrolo[2,3-d]pyrimidin-6(7H)-one-5-yl; 4-carboxypiperidin-1-yl; or
piperazin-1-yl substituted with R⁴, R⁵ or R⁶ at the 4-position of the piperazin-1-yl ring where R⁴, R⁵ or R⁶ are as defined above or where R⁴, R⁵ or R⁶ are hydrogen, alkoxycarbonyl, or —CONHR where R is phenyl substituted with alkoxy, cyano, alkyl, 5-hydroxyindol-1-yl, or cyclopropyl;
(ii) when X and Z are nitrogen, R¹ is hydrogen, R² is cycloalkylpropoxy, R³ is alkoxy, then and R^{3a} is not piperazin-1-yl substituted with R⁴, R⁵ or R⁶ where two of R⁴, R⁵ or R⁶ are hydrogen and the other of R⁴, R⁵ or R⁶ is at the 4-position of the piperazin-1-yl ring and is hydrogen or —CONHR where R is phenyl substituted with alkoxy; and
(iii) when X and Z are nitrogen, R¹ is hydrogen, R² is 2-(dimethylamino)ethoxy, and R³ is methoxy, then R^{3a} is not 1,6-dimethyl-2,4-dioxo-1,2-dihydroquinazolin-3(4H)-yl-piperidin-1-yl; or a salt of (i)-(iii).

2. The compound of claim 1 wherein X and Y are nitrogen and Z is ═CH—.

3. The compound of claim 1 wherein Y and Z are nitrogen and X is —CH═.

4. The compound of claim 1 wherein X and Z are nitrogen and Y is ═CH.

5. The compound of claim 2 wherein R¹ is hydrogen, one of R² and R³ is alkoxy and the other is cycloalkoxy.

6. The compound of claim 2 wherein R¹ is hydrogen, one of R² and R³ is alkoxy and the other is hydroxyalkyloxy or alkoxyalkyloxy.

7. The compound of claim 2 wherein R¹ is hydrogen, one of R² and R³ is alkoxy and the other is —O-(alkylene)-NR¹⁵R¹⁶.

8. The compound of claim 2 wherein R¹ is hydrogen, one of R² and R³ is alkoxy and the other is alkoxyalkoxy, and R^{3a} is a ring of formula: 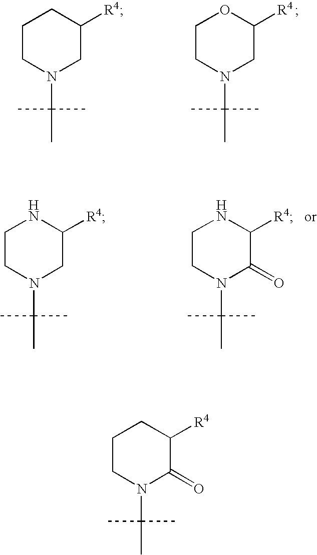
where R⁴ phenyl, heteroaryl, or six membered saturated heterocyclyl optionally substituted with R^{a}, R^{b} and R^{c} and the rings are substituted, including the hydrogen atom on the —NH— group within the ring, with R⁵ and R⁶.

9. The compound of claim 2 wherein R¹ is hydrogen, one of R² and R³ is alkyl and the other is hydroxyalkoxy or alkoxyalkoxy, and R^{3a} is a ring of formula: 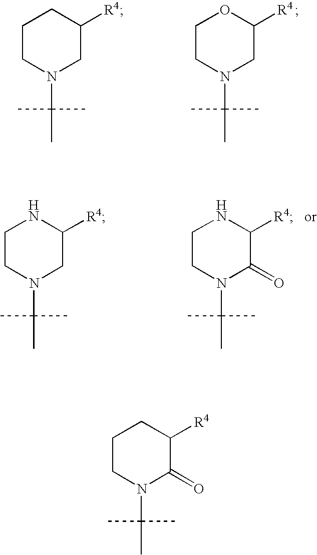
where R⁴ is phenyl, heteroaryl, or six membered saturated heterocyclyl optionally substituted with R^{a}, R^{b} and R^{c} and the rings are substituted, including the hydrogen atom on the —NH— group within the ring, with R⁵ and R⁶.

10. The compound of claim 2 wherein R¹ is hydrogen, one of R² and R³ is alkoxy and the other is —O-(alkylene)-NR¹⁵R¹⁶, and R^{3a} is a ring of formula: 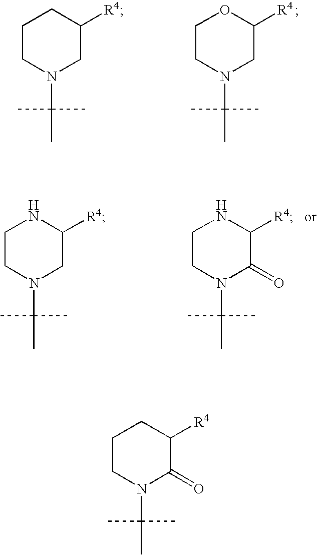
where R⁴ is phenyl, heteroaryl, or six membered saturated heterocyclyl optionally substituted with R^{a}, R^{b} and R^{c} and the rings are substituted, including the hydrogen atom on the —NH— group within the ring, with R⁵ and R⁶.

11. The compound of claim 2 wherein R¹ is hydrogen, one of R² and R³ is alkoxy and the other is alkoxyalkoxy, and R^{3a} is a ring of formula:

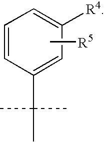

12. The compound of claim 2 wherein R¹ is hydrogen, one of R² and R³ is alkyl and the other is hydroxyalkoxy or alkoxyalkyloxy, and R^{3a} is a ring of formula:

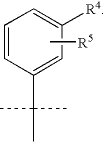

13. The compound of claim 2 wherein R¹ is hydrogen, one of R² and R³ is alkoxy and the other is alkoxyalkoxy, and R^{3a} is a ring of formula:

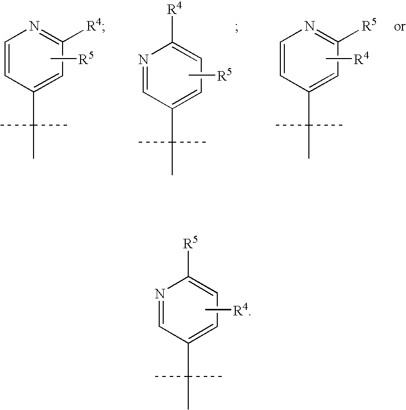

14. The compound of claim 2 wherein R¹ is hydrogen, one of R² and R³ is alkyl and the other is hydroxyalkoxy or alkoxyalkyloxy, and R^{3a} is a ring of formula:

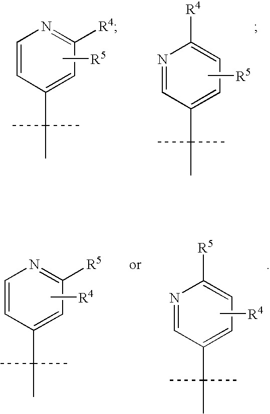

15. The compound of claim 2 wherein R¹ is hydrogen, one of R² and R³ is alkyl and the other is hydroxyalkoxy or alkoxyalkyloxy, and R^{3a} is a ring of formula: 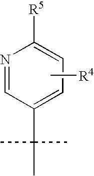
where R⁵ is monosubstituted or disubstituted amino and R⁴ is hydrogen, alkyl, or halo.

16. The compound of claim 2 wherein R¹ is hydrogen, one of R² and R³ is alkoxy and the other is hydroxyalkoxy or alkoxyalkyloxy, and R^{3a} is a ring of formula: 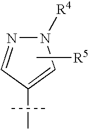
where R⁵ is hydrogen or alkyl and R⁴ is aryl, heteroaryl, aralkyl, heteroaralkyl, or heterocyclyl optionally substituted with one to three substitutents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, or optionally substituted heterocyclyl.

17. The compound of claim 2 wherein R¹ is hydrogen, one of R² and R³ is alkyl and the other is hydroxyalkoxy or alkoxyalkyloxy, and R^{3a} is a ring of formula: 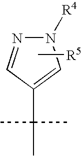
where R⁵ is hydrogen or alkyl and R⁴ is aryl, heteroaryl, aralkyl, heteroaralkyl, or heterocyclyl optionally substituted with one to three substitutents independently selected from R^{a}, R^{b}, and R^{c} which are alkyl, cycloalkyl, cycloalkylalkyl, cycloalkoxy, cycloalkylalkyloxy, alkoxy, halo, haloalkyl, haloalkoxy, hydroxyl, hydroxyalkyl, alkoxyalkyl, hydroxyalkoxy, alkoxyalkyloxy, aminoalkyl, aminoalkoxy, acyl, cyano, carboxy, alkoxycarbonyl, alkylthio, sulfinyl, sulfonyl, aminocarbonyl, aminosulfonyl, monosubstituted amino, disubstituted amino, optionally substituted phenyl, optionally substituted heteroaryl, or optionally substituted heterocyclyl.

18. A pharmaceutical composition comprising a compound of claim 1 and a pharmaceutically acceptable expicient.

19. A method of treating a disorder treatable by inhibition of PDE10 enzyme in a patient which method comprises administering to the patient a pharmaceutical composition comprising a a compound of claim 1 and a pharmaceutically acceptable expicient.

20. The method of claim 18 wherein the disease is schizophrenia, bipolar disorder, or obsessive-compulsive disorder.
